(19) United States
(12) Patent Application Publication
(10) Pub. No.: US 2024/0261303 A1
(43) Pub. Date: Aug. 8, 2024

(54) PARTICLES, AQUEOUS DISPERSIONS, AND COMPOSITIONS HAVING HIGH LIPOPHILIC COMPONENT CONCENTRATIONS AND HIGH LIPOPHILIC COMPONENT TO SURFACTANT RATIOS

(71) Applicant: Spoke Sciences, Inc., Bainbridge Island, WA (US)

(72) Inventors: Mark Mitchnick, East Hampton, NY (US); Brad Douglass, Black Diamond, WA (US); Kimberly Ann Zubris, Alburtis, PA (US)

(21) Appl. No.: 18/421,851

(22) Filed: Jan. 24, 2024

Related U.S. Application Data
(63) Continuation-in-part of application No. PCT/US2022/051563, filed on Dec. 1, 2022.
(60) Provisional application No. 63/284,921, filed on Dec. 1, 2021.

(51) Int. Cl. A61K 31/00; A61K 9/00; A61K 9/16; A61K 36/185; A61K 47/22; A61K 47/26; A61K 47/46

(52) U.S. Cl. CPC A61K 31/658; A61K 9/0095; A61K 9/1617; A61K 9/1623; A61K 36/185; A61K 47/22; A61K 47/26; A61K 47/46

(57) ABSTRACT

Particles, aqueous dispersions, and compositions having high lipophilic component (LC) concentrations and high LC to surfactant ratios are described. The particles, aqueous dispersions, and compositions can be used to efficiently deliver active compounds and/or active ingredients through a variety of formulation types.

### CROSS REFERENCE TO RELATED APPLICATION

This application is a continuation-in-part of International Patent Application No. PCT/US2022/051563, filed on Dec. 1, 2022, which claims priority to U.S. Provisional Patent Application No. 63/284,921, filed on Dec. 1, 2021, the contents of both of which are incorporated by reference in their entirety as if fully set forth herein.

### FIELD OF THE DISCLOSURE

The present disclosure provides particles, aqueous dispersions, and compositions having high lipophilic component (LC) concentrations and high LC to surfactant ratios. The particles, aqueous dispersions, and compositions can be used to efficiently deliver active compounds and/or active ingredients.

### BACKGROUND OF THE DISCLOSURE

A number of lipophilic components (LCs) that have beneficial physiological effects are difficult to formulate into liquid carriers for administration. Cannabinoids are one such LC, having a water-solubility of less than 1 μg/mL.

Attempts to prepare oral formulations of cannabinoids have involved preparing capsules or formulations in alcohol or using various emulsification approaches that require large amounts of carriers and surfactants. Unfortunately, these compositions can contain an unhealthy amount of non-active ingredients, and can have low bioavailability, and a short shelf-life. Furthermore, for use in a pediatric setting, it is desirable to have little to no ethanol in formulations. Capsules can also be difficult for young children to swallow. A need therefore exists for improved methods to deliver cannabinoids as well as other LCs.

### SUMMARY OF THE DISCLOSURE

The current disclosure provides particles, aqueous dispersions, and compositions having high lipophilic component (LC) concentrations and high LC to surfactant ratios. The particles, aqueous dispersions, and compositions can be used to efficiently deliver active compounds and active ingredients, such as cannabinoids.

In certain other examples, the current disclosure provides lipid particles including (i) at least two LCs and (ii) a surfactant, wherein at least one LC is solid at room temperature, and at least a percentage of the surfactant is on the surface of the lipid particle. Within these particles, the two LCs can be derived from a common plant type (e.g., cannabis) to prepare a particle with a high concentration of components from the common plant type. Within these particles, at least one LC and the surfactant can be derived from a common plant type (e.g., cannabis) to prepare a particle with a high concentration of components from a common plant type.

In certain other examples, the current disclosure provides a lipid particle including (i) at least two LCs, (ii) a surfactant, and (iii) a preservative wherein at least one LC or preservative is solid at room temperature, and at least a percentage of the surfactant and preservative is on the surface of the lipid particle.

In certain other examples, the current disclosure provides a lipid particle including (i) at least two LCs, (ii) a surfactant, and (iii) at least one stabilizer, wherein the stabilizer is solid at room temperature and at least a percentage of the stabilizer is on the surface of the lipid particle.

In certain other examples, the current disclosure provides a lipid particle including (i) at least one LC that is both an active compound and either a carrier or a viscosity modifier and (ii) a surfactant, wherein the at least one LC is solid at room temperature, and at least a percentage of the surfactant is on the surface of the lipid particle.

In certain other examples, the current disclosure provides a lipid particle including (i) an LC, (ii) a surfactant, and (iii) a glycoside. In certain other examples, the current disclosure provides a lipid particle including (i) an LC, (ii) a surfactant, and (iii) at least two glycosides.

In certain examples, the current disclosure provides an aqueous dispersion or liquid composition including lipid particles wherein the lipid particles include (i) a cannabinoid and (ii) a surfactant, wherein the ratio of the cannabinoid to the surfactant within the aqueous dispersion or liquid composition is at least 1:1 (% w/w).

In certain examples, the current disclosure provides an aqueous dispersion or liquid composition including lipid particles wherein the lipid particles include (i) an LC, (ii) a surfactant, and (iii) a glycoside wherein the ratio of the LC to glycoside within the aqueous dispersion or liquid composition is at least 5:1 (% w/w), 6:1 (% w/w), 7:1 (% w/w), 8:1 (% w/w), 9:1 (% w/w), or 10:1 (% w/w).

In certain other examples, the current disclosure provides an aqueous dispersion or liquid composition including (i) lipid particles dispersed in (ii) an aqueous media, wherein the lipid particles contain a surfactant and at least two LCs in which at least one of the LCs is an active compound. In particular embodiments, the active compound is a cannabinoid. In particular embodiments, the aqueous dispersion or liquid composition includes over 100 mg of a cannabinoid in 1 mL of the aqueous media.

In certain other examples, the current disclosure provides an aqueous dispersion or liquid composition including (i) lipid particles dispersed in (ii) an aqueous media, wherein the lipid particles contain a surfactant and at least two LCs in which at least one of the LCs is an active compound and wherein the aqueous dispersion or liquid composition includes over 100 mg of the active compound in 1 mL of the aqueous media, and wherein the aqueous dispersion or liquid composition is diluted with an additional aqueous phase to a total active compound concentration of less than 10 mg per mL of total aqueous media.

In certain other examples, the current disclosure provides an aqueous dispersion or liquid composition including (i) lipid particles dispersed in (ii) an aqueous media, wherein the lipid particles contain a surfactant and at least two LCs in which one LC is an active compound, and wherein the aqueous dispersion or liquid composition includes over 100 mg of the active compound in 1 mL of the aqueous media, and wherein the aqueous dispersion or liquid composition is diluted with an additional aqueous phase to a total active compound concentration of less than 10 mg per mL of total aqueous media and then dehydrated to produce either a second aqueous dispersion or dehydrated solid with a total active compound concentration greater than 10 mg per gram (i.e. 1% by weight) of total active compound.

In certain other examples, the current disclosure provides an aqueous dispersion or liquid composition including (i) lipid particles dispersed in (ii) an aqueous media, wherein the lipid particles contain a surfactant and at least two LCs in which one LC is an active compound, and wherein the one active compound is a cannabinoid present in the aqueous dispersion or liquid composition in a concentration over 100 mg of the cannabinoid in 1 mL of the aqueous media.

In certain other examples, the current disclosure provides an aqueous dispersion or liquid composition including (i) lipid particles dispersed in (ii) an aqueous media, wherein the lipid particles contain a surfactant and an active compound, and wherein the active compound is present in the aqueous dispersion or liquid composition in a concentration over 100 mg of the active compound in 1 mL of the aqueous media. In particular embodiments, the active compound is a cannabinoid.

In certain other examples, the current disclosure provides an aqueous dispersion or liquid composition including (i) lipid particles dispersed in (ii) an aqueous media, wherein the lipid particles contain a surfactant, at least two LC, and a glycoside, in which at least one of the LCs is an active compound.

In certain other examples, the current disclosure provides an aqueous dispersion or liquid composition including (i) lipid particles dispersed in (ii) an aqueous media, wherein the lipid particles contain a surfactant, at least two LC, and at least two glycosides, in which at least one of the LCs is an active compound.

Typically, smaller particles for lipid-in-water emulsions and dispersions lead to a number of occurrences in direct proportion to decreasing particle size: (1) a requirement for more surfactant(s) often in relative ratios of 1:1 to 1:10 lipophilic-to surfactant to generate a stable emulsion or dispersion, (2) decreased palatability due to both the increasing surfactant content and the smaller particles facilitating increased interaction with chemoreceptors causing the particles to taste bitter, and (3) less protection from environmental insults, such as heat, light and oxidation that can lead to the chemical degradation of the active ingredient(s) of the emulsion or dispersion. As described herein, it was discovered that the particles described herein did not exhibit the decreased palatability nor protection issues as a result of decreasing particle size. Furthermore, it was unexpectedly determined that the particles of the instant disclosure require relative ratios of at least 2:1 lipophilic-to-surfactant, which is well below the amount of surfactant required by typical emulsions and liquid dispersions. In particular embodiments, the lipophilic-to surfactant ratio is 3:1 (% w/w) or greater. Having achieved this summary of described advances in relation to the delivery of cannabinoids in liquid carriers, such as aqueous dispersions, the advances can be applied in a variety of alternatives and contexts described in more detail throughout the following disclosure.

### BRIEF DESCRIPTION OF THE FIGURES

Some of the drawings submitted herein may be better understood in color. Applicant considers the color versions of the drawings as part of the original submission and reserves the right to present color images of the drawings in later proceedings.

FIG. 1. Formulation composition and manufacturing procedure. Lipid phase ingredients were weighed and heated until liquified and 70° C. was reached. Then, the aqueous phase ingredients were weighed and heated until they reached 75° C. Once both phases reached 70-75° C., the aqueous phase was added to the lipid phase and the mixture was rapidly sonicated for 15 minutes at 90% amplitude while stirring and heating on a hotplate. Next, the formulation was transferred to an ice bath and stirred for 15 minutes to reach room temperature. The formulations here are at a ratio of 1.8:1 and 1.25:1 for CBD: surfactant.

FIG. 2. Photograph of aqueous dispersion of lipid particles at T₀. No sedimentation or separation of particles was observed in any of the four samples at T₀.

FIGS. 3A-3E. RG1. (3A) Table of formulation composition for RG1. (3B) Table of particle size distribution. The ratio of CBD: surfactant is 1.25:1. (3C, 3D) Optical microscopy of RG1 at 400× magnification under normal light (3C) and (3D) polarized light. (3E) Particle size distribution (PSD) by number and volume.

FIGS. 4A-4E. RG3. (4A) Table of formulation composition for RG3. (4B) Table of particle size distribution. The ratio of CBD: surfactant is 1.25:1. (4C, 4D) Optical microscopy of RG3 at 400× magnification under normal light (4C) and (4D) polarized light. (4E) PSD by number and volume.

FIGS. 5A-5E. G4. (5A) Table of formulation composition for G4. (5E) Table of particle size distribution. The ratio of CBD: surfactant is 1.8:1. (5C) Optical microscopy of G4 at 400× magnification under normal light and (5D) polarized light. (5E) PSD by number and volume.

FIGS. 6A-6E. G5. (6A) Table of formulation composition for G5. (6E) Table of particle size distribution. The ratio of CBD: surfactant is 1.8:1. (6C) Optical microscopy of G5 at 400× magnification under normal light and (6D) polarized light. (6E) PSD by number and volume.

FIG. 7A-7E. Optical microscopy and informal physical stability of G1, G2 and G3 at week 1 (T1 week). Optical microscopy at 400× magnification under normal light for (7A) G1 (T1 week), (7E) G2 (T1 week), and (7C) G3 (T1 week). No CBD separation is observed at T1 week in optical microscopy. (7D) Some sedimentation is observed in G1, G2 and G3 due to larger particle size distribution, but they redisperse very quickly. (7E) Table of formulation compositions for G1, G2, and G3. The ratios of CBD:surfactant are 1.8:1, 3.6:1, and 1.8:1 for G1, G2, and G3, respectively.

FIG. 8. Solidification of Carnauba wax+Limonene mixtures. Mixtures were heated and transferred to bench top for ambient stirring for 30 minutes where solidification was observed in all of the samples. No melting of carnauba wax was observed at 50° C. Heating temperature above 70° C. was required to melt carnauba wax in the mixture. The most solid mixture at room temperature is Carnauba wax: Limonene in ratio of 2:1 (The difference between solidified physical structure of samples is minute).

FIG. 9. Laser scattering PSD analyzer shows a median particle size of 1.38945 μm and a mean particle size of 3.13952 μm.

FIG. 10. Exemplary structures of cannabinoids including CBC, CBN, CBCV, CBNV, CBD, CBT, CBDA, THC, CBDV, THCA, CBG, THCV, CBGV, THCVA, and CBL.

FIG. 11. Exemplary synthetic and/or artificial cannabinoids including nabilone, 7-OH-CBD, 7-OH-CBDV, and Formula (I) to Formula (XVI).

FIGS. 12A-12C. SKS-23H. (12A) Table of formulation composition for SKS-23H. The ratio of CBD: surfactant is 3.6:1. (12E) Photograph of aqueous dispersion of SKS-23H at T0. (12C) Optical microscopy of SKS-23H under normal light.

FIGS. 13A-13C. SKS-31H. (13A) Table of formulation composition for SKS-31H. (13E) Photograph of aqueous dispersion of SKS-31H at T0. (13C) Optical microscopy of SKS-31H under normal light.

FIGS. 14A-14C. SKS-33H. (14A) Table of formulation composition for SKS-33H. (14E) Photograph of aqueous dispersion of SKS-33H at T0. (14C) Optical microscopy of SKS-33H under normal light.

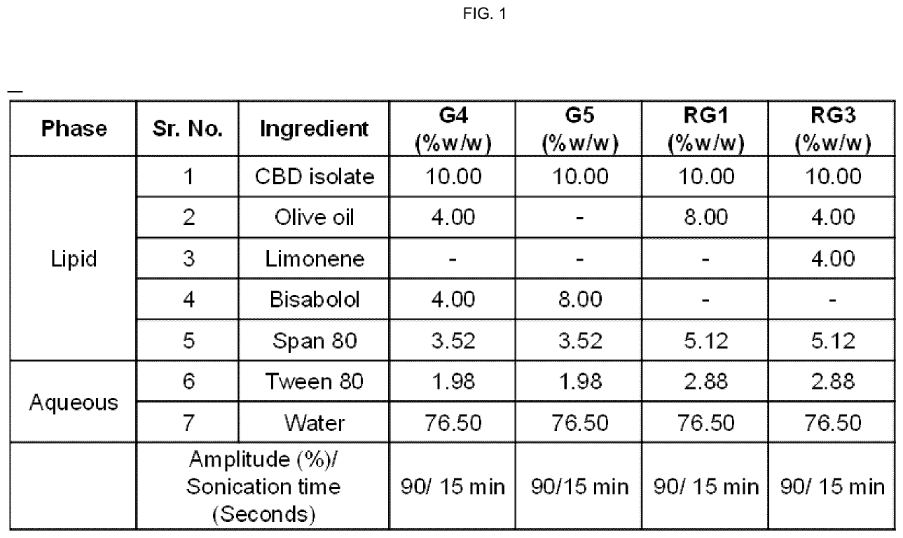

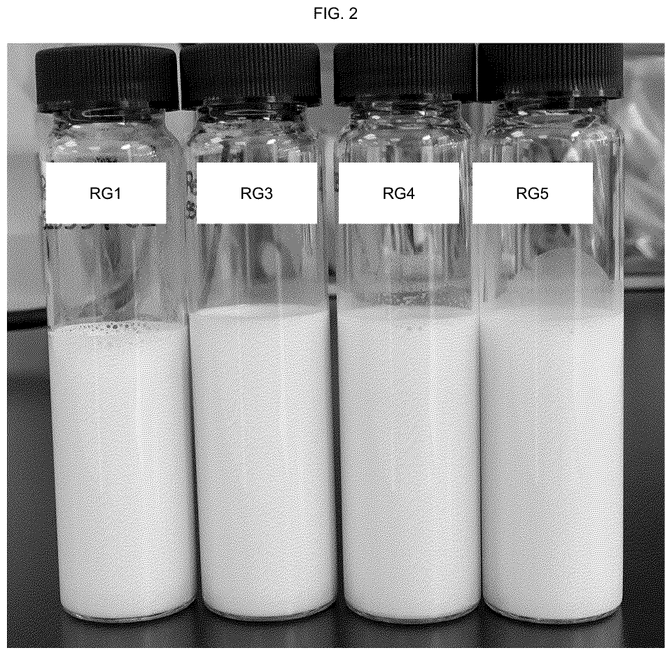

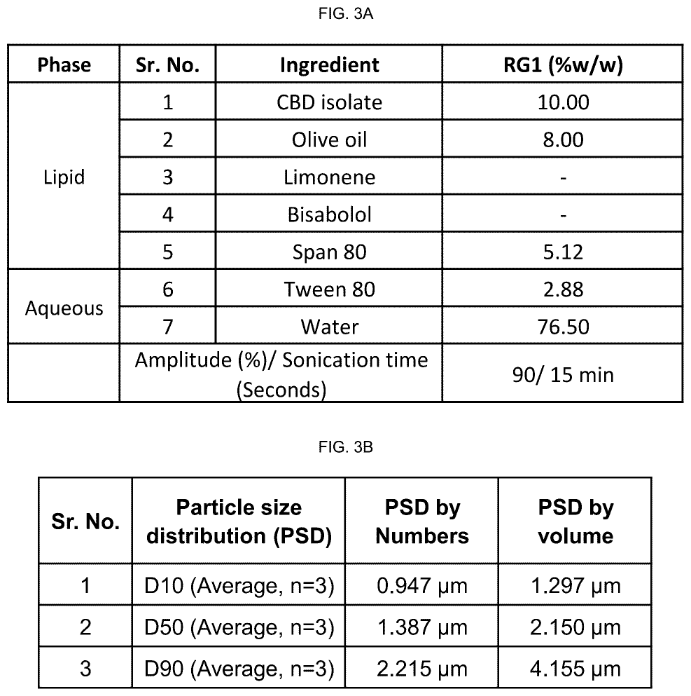

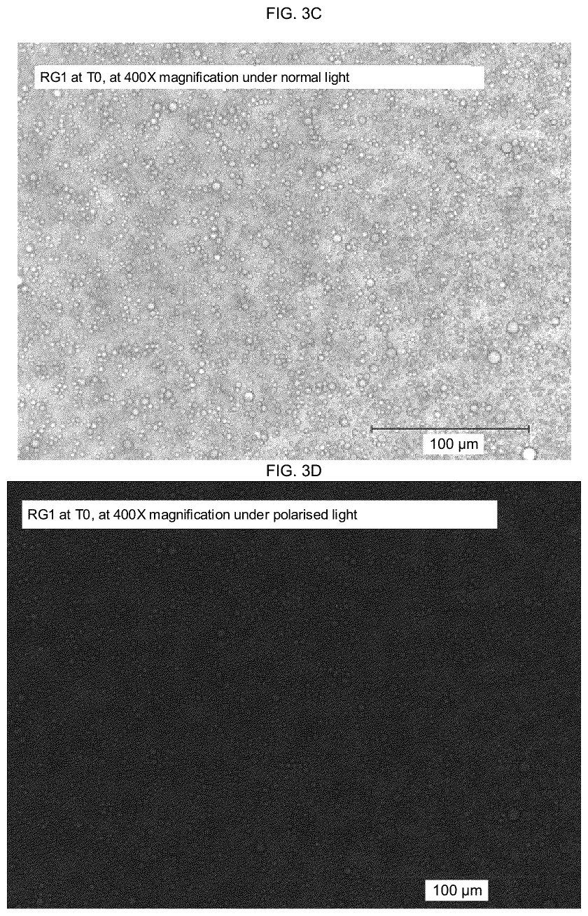

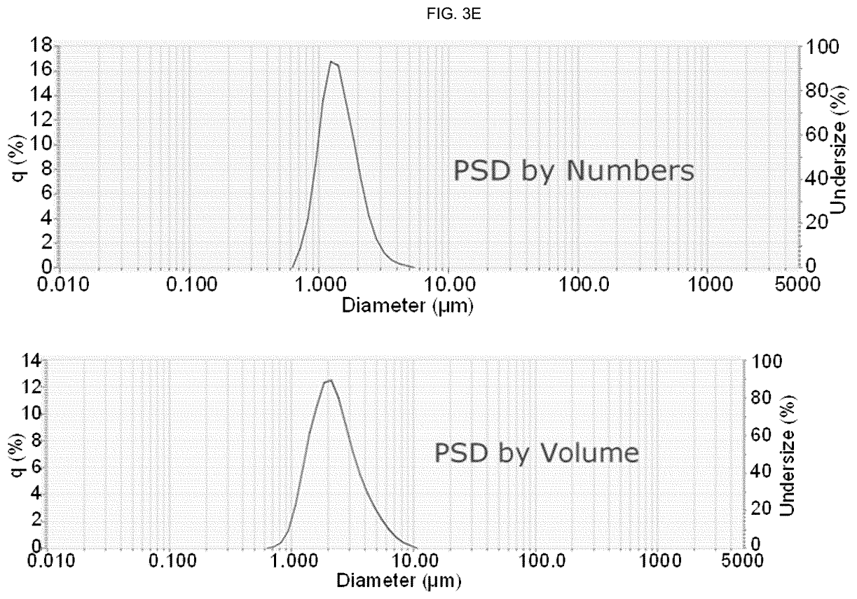

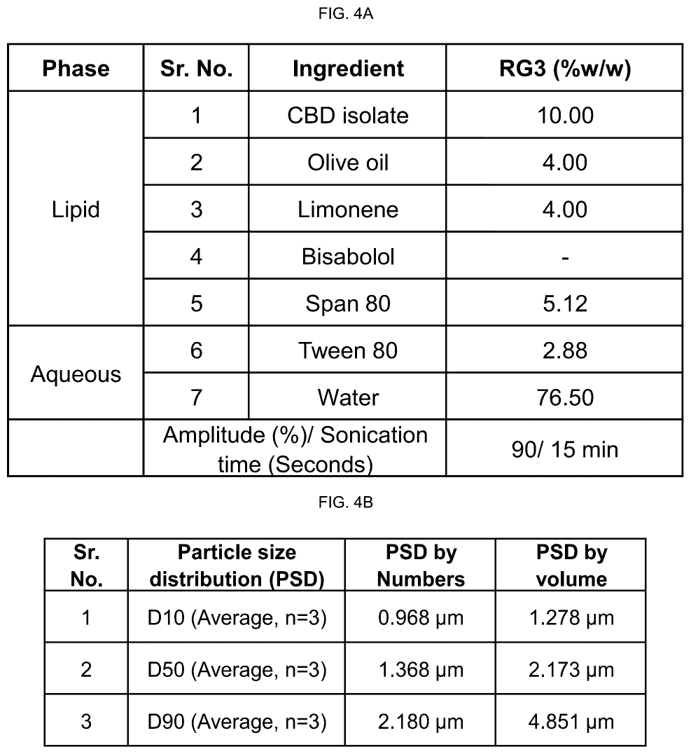

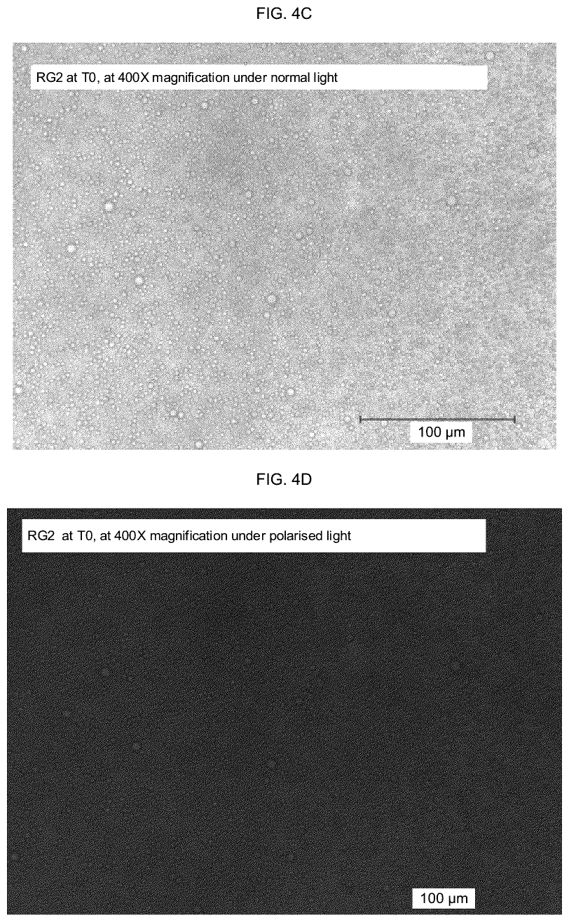

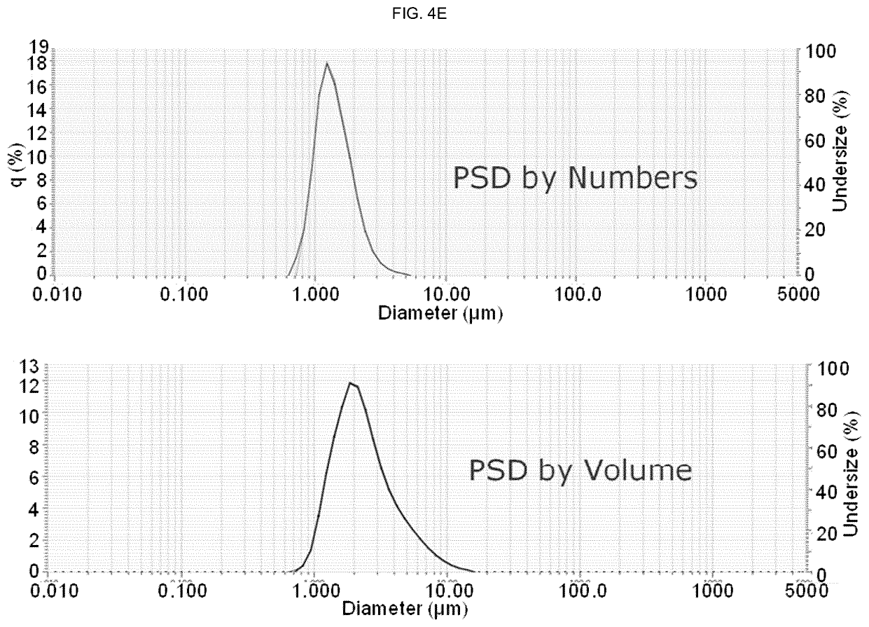

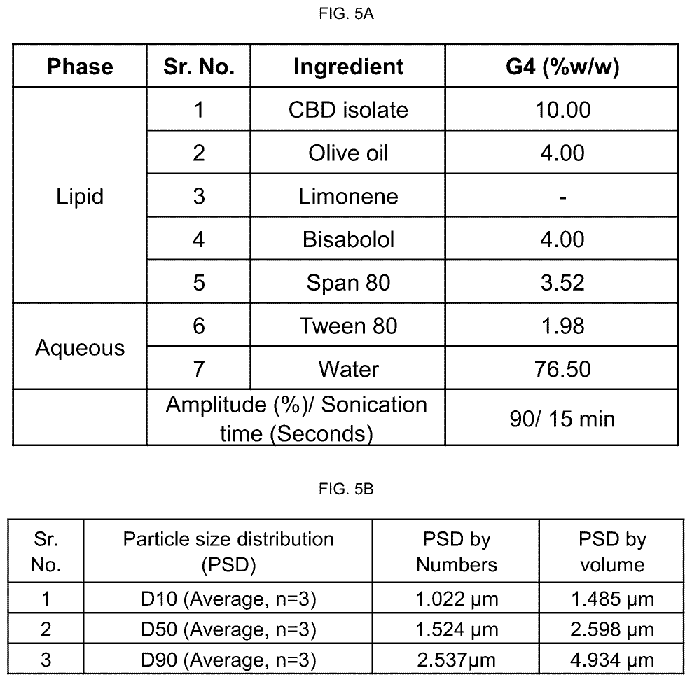

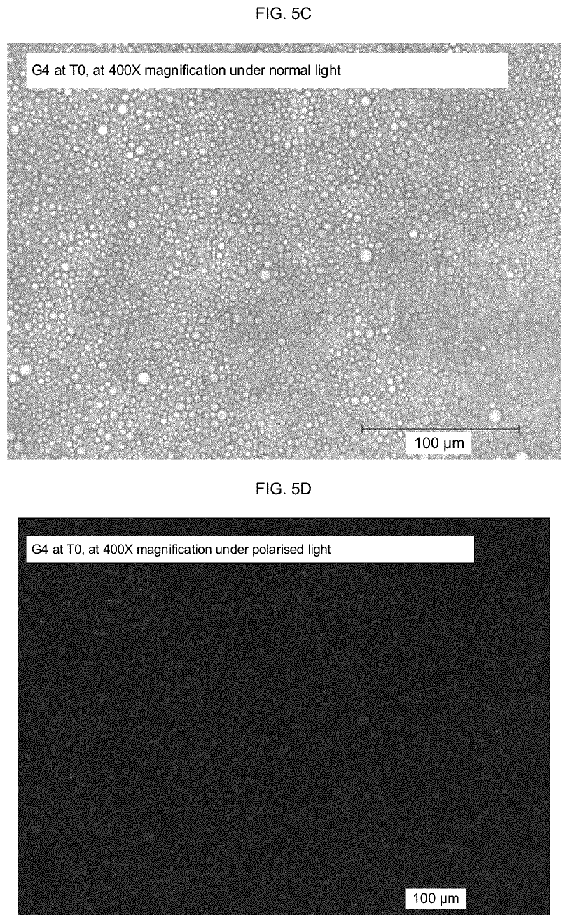

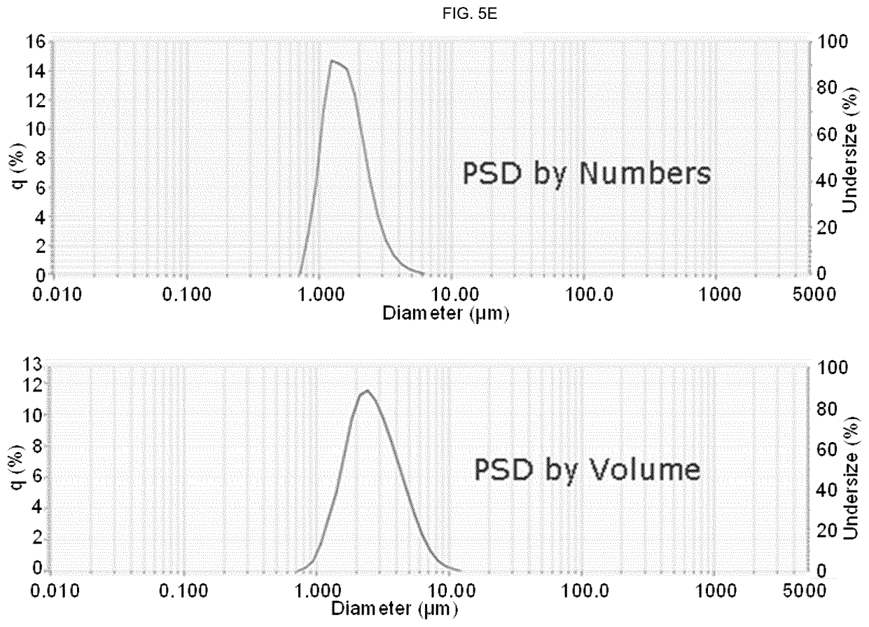

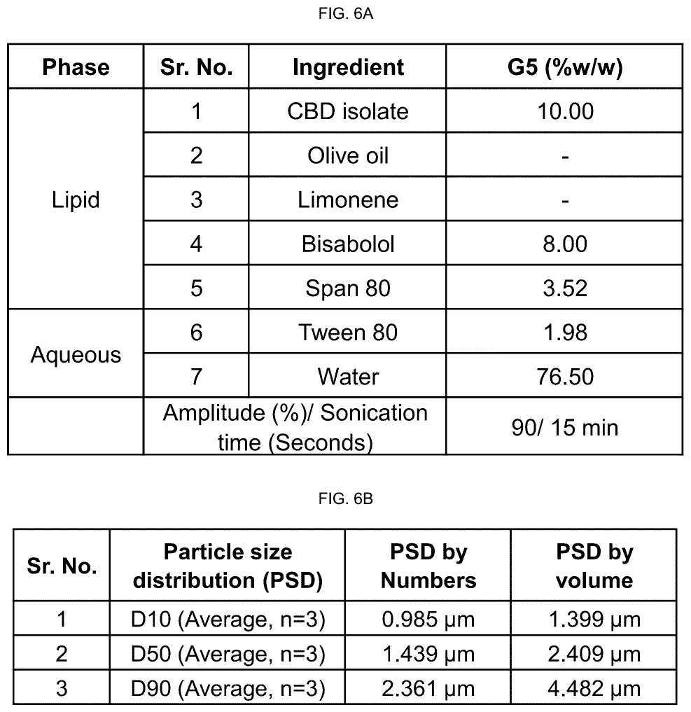

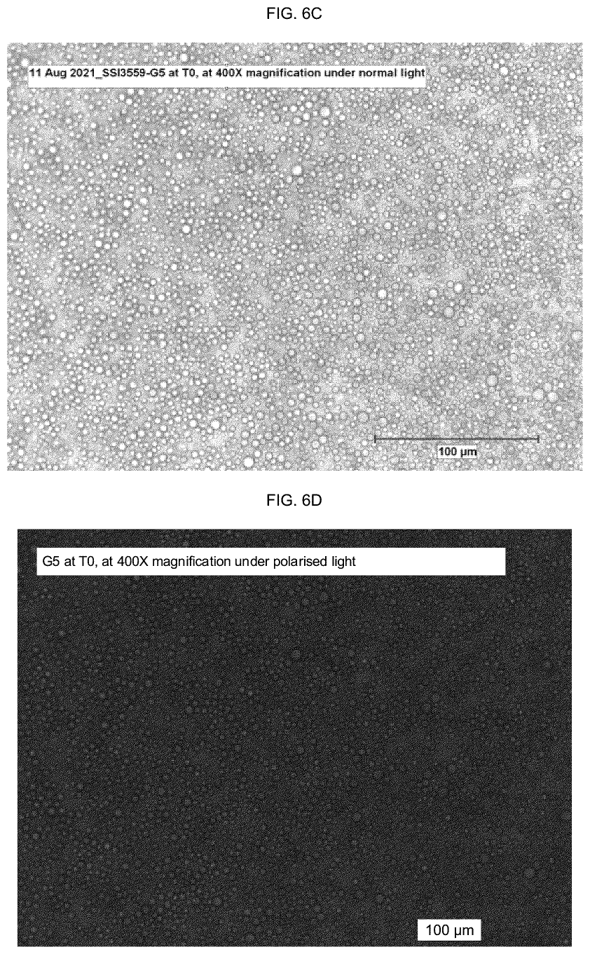

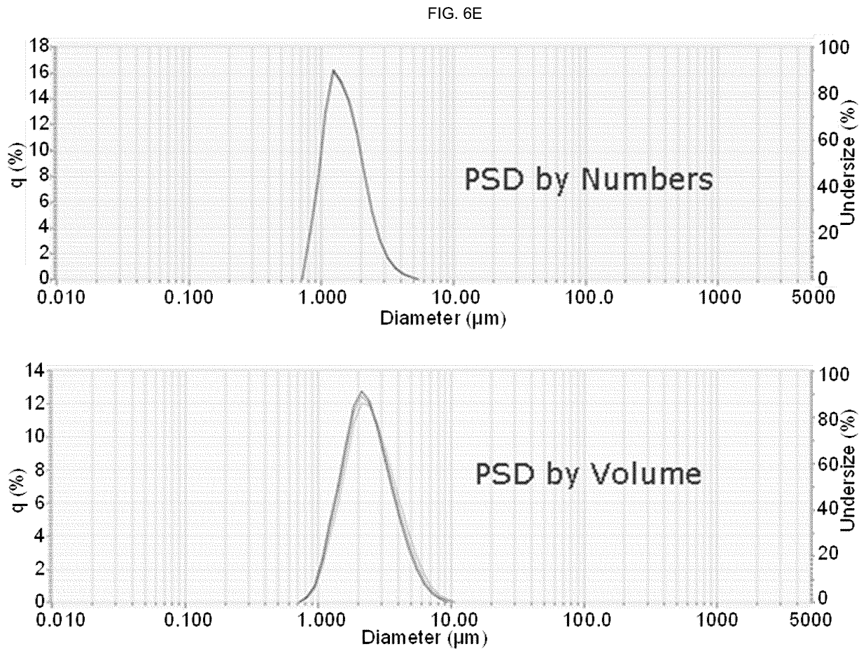

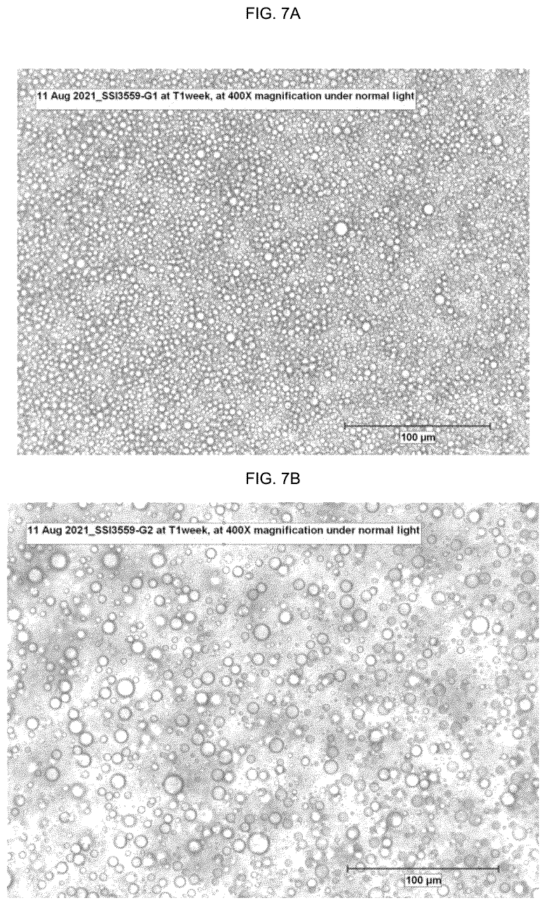

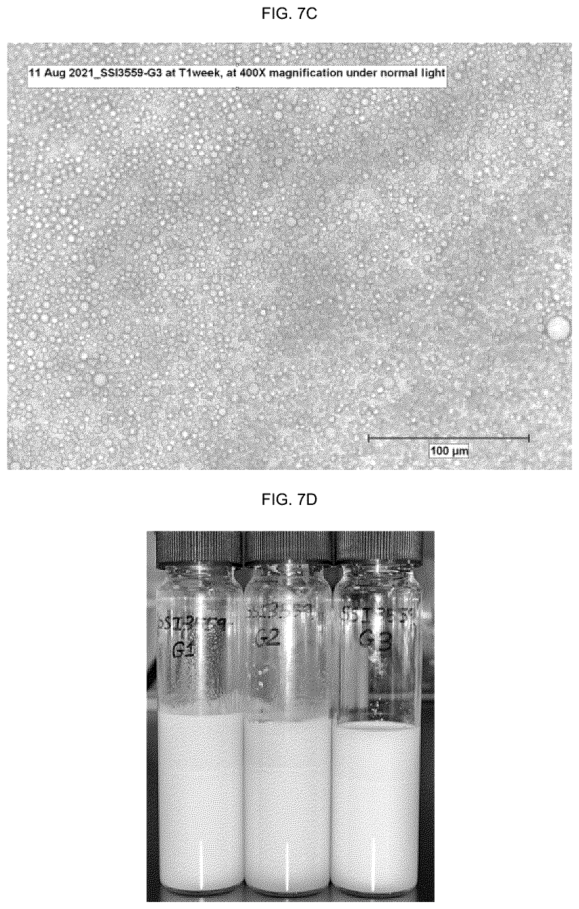

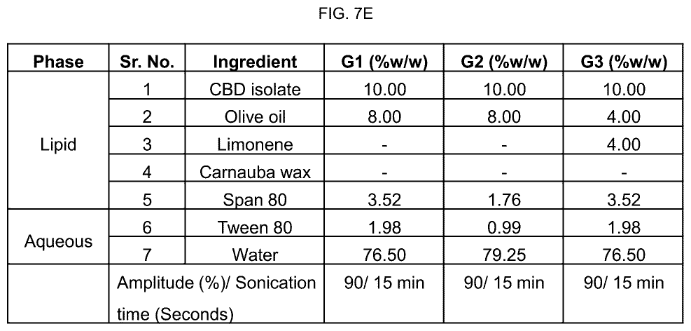

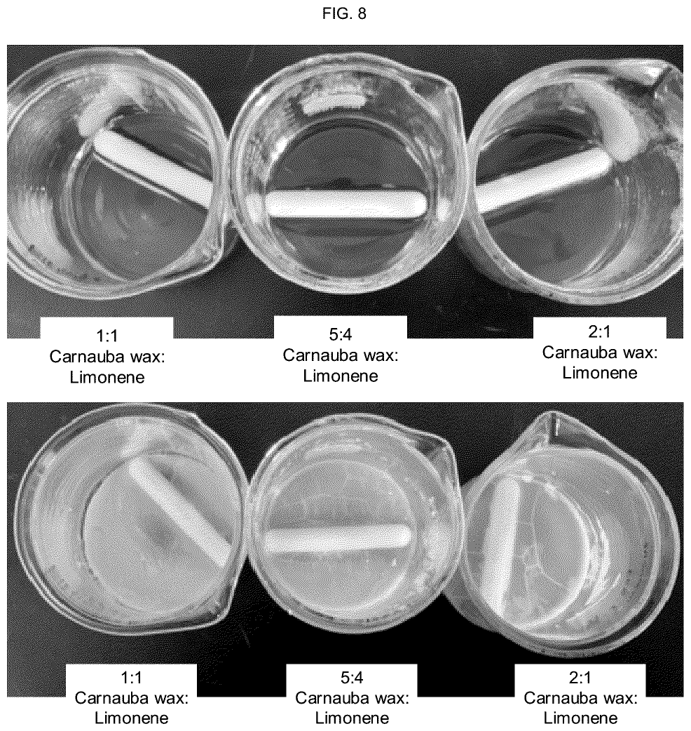

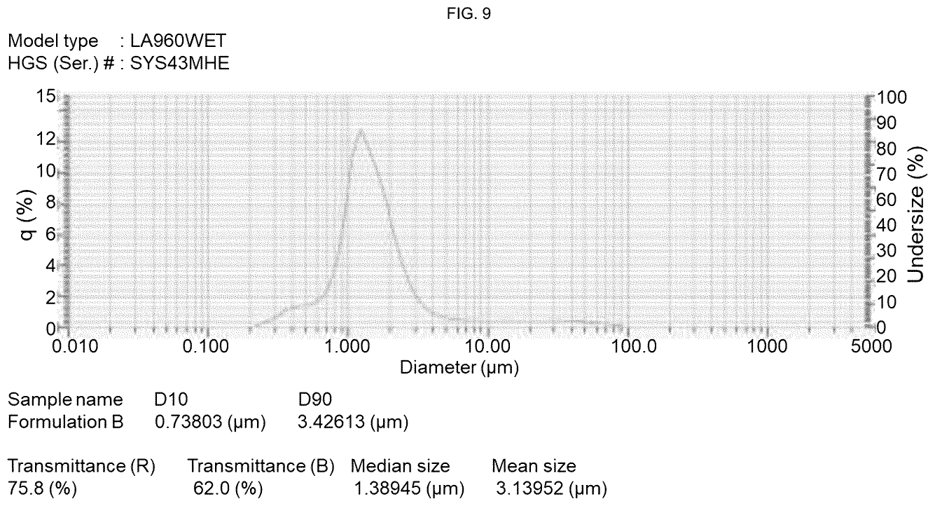

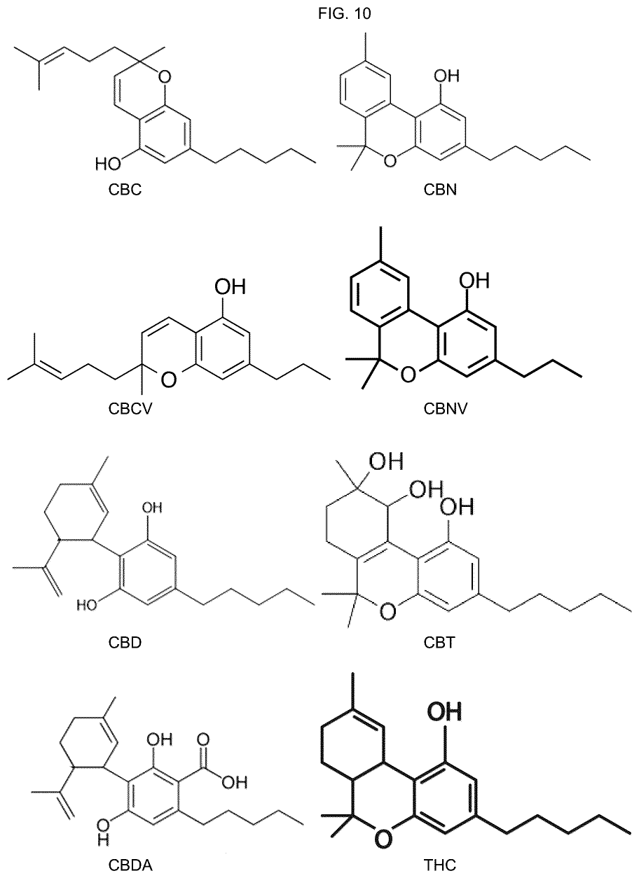

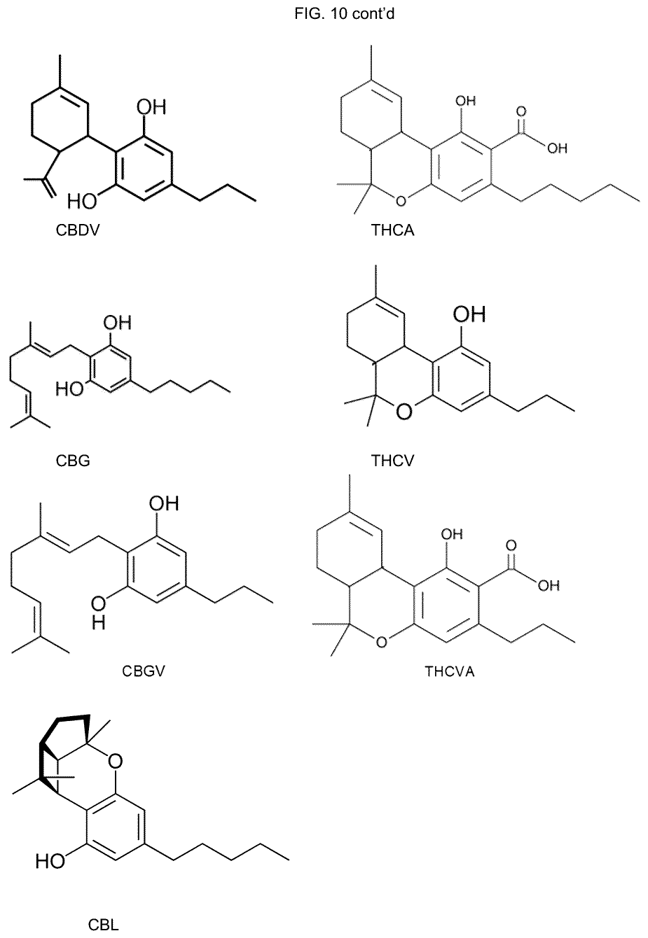

### DETAILED DESCRIPTION

A number of lipophilic components (LCs) that have beneficial physiological effects are difficult to formulate into liquid carriers for administration. Cannabinoids are one such LC, having a water-solubility of less than 1 μg/mL.

Attempts to prepare oral formulations of cannabinoids have involved preparing capsules or formulations in alcohol or using various emulsification approaches that require large amounts of carriers and surfactants. Unfortunately, these compositions can contain an unhealthy amount of non-active ingredients, and can have low bioavailability, and a short shelf-life. Furthermore, for use in a pediatric setting, it is desirable to have little to no ethanol in formulations. Capsules can also be difficult for young children to swallow. A need therefore exists for improved methods to deliver cannabinoids and other lipophilic components.

The current disclosure provides particles, aqueous dispersions, and compositions having high LC concentrations and high LC to surfactant ratios. The particles, aqueous dispersions and compositions can be used to efficiently deliver LC. An LC refers to a compound that has the ability to at least partially dissolve in lipids and non-polar solvents. In particular embodiments, an LC is a lipid active compound (LAC). LAC have an intended physiological effect in the body. In certain examples, LAC can be regulated LAC (rLAC). rLAC have an intended physiological effect in the body and are also subject to government approval by a regulatory body, such as the U.S. Food & Drug Agency (FDA). LC have an affinity for lipids. Lipophilic components generally include oils, fatty esters, hydrocarbon oils, silicones, waxes, fatty alcohols, lipophilic vitamins, some proteins, and phospholipids. Within the context of the current disclosure, hydrophobic fluorocarbons are considered lipophilic components.

As indicated, particles disclosed herein include a high LC concentration. In certain examples, the LC is a cannabinoid and its concentration within the particle is at least 10% w/w.

In particular embodiments, lipid particles disclosed herein include 35-55% w/w first LC, 10-45% w/w second LC, and 8-30% w/w surfactant, wherein the selected amounts do not collectively exceed 100% w/w.

In particular embodiments, lipid particles disclosed herein include 40-50% w/w first LC, 12-40% w/w second LC, and 10-25% w/w surfactant, wherein the selected amounts do not collectively exceed 100% w/w.

In particular embodiments, lipid particles disclosed herein include 41-49% w/w first LC, 15-40% w/w second LC, and 10-25% w/w surfactant, wherein the selected amounts do not collectively exceed 100% w/w.

In particular embodiments, lipid particles disclosed herein include 42-48% w/w first LC, 16-39% w/w second LC, and 12-24% w/w surfactant, wherein the selected amounts do not collectively exceed 100% w/w.

In particular embodiments, lipid particles disclosed herein include 66.7-87% w/w first LC and second LC, and 13-33.3% w/w surfactant, wherein the selected amounts do not collectively exceed 100% w/w.

In particular embodiments, lipid particles disclosed herein include 48-49% (e.g., 48.2%) w/w first LC, 38-39% (e.g., 38.5%) w/w second LC, and 13-14% (e.g., 13.3%) w/w surfactant wherein the selected amounts do not collectively exceed 100% w/w.

In particular embodiments, lipid particles disclosed herein include 35-90% w/w first LC and second LC, 0.001-30% w/w surfactant, and 0.001-40% w/w glycoside wherein the selected amounts do not collectively exceed 100% w/w. In particular embodiments, lipid particles disclosed herein include 20-90% w/w first LC and second LC, 3-20% w/w surfactant, and 0.5-20% w/w glycoside wherein the selected amounts do not collectively exceed 100% w/w. In particular embodiments, the lipid particles include at least two glycosides.

In particular embodiments, lipid particles disclosed herein include 42-43% (e.g., 42.6%) w/w first LC, 16-18% (e.g., 17%) w/w second LC, 16-18% (17%) w/w third LC, and 23-24% (e.g., 23.4%) w/w surfactant wherein the selected amounts do not collectively exceed 100% w/w.

In particular embodiments, lipid particles disclosed herein include 35-90% w/w first LC, second LC, and third LC; 0.001-30% w/w surfactant; and 0.001-40% w/w glycoside wherein the selected amounts do not collectively exceed 100% w/w. In particular embodiments, lipid particles disclosed herein include 20-90% w/w first LC, second LC, and third LC; 3-20% w/w surfactant; and 0.5-20% w/w glycoside wherein the selected amounts do not collectively exceed 100% w/w. In particular embodiments, the lipid particles include at least two glycosides.

In particular embodiments, lipid particles disclosed herein include 35-55% w/w cannabinoid, 10-45% w/w olive oil or sesame oil, and 8-30% w/w surfactant, wherein the selected amounts do not collectively exceed 100% w/w.

In particular embodiments, lipid particles disclosed herein include 40-50% w/w cannabinoid, 12-40% w/w olive oil or sesame oil, and 10-25% w/w surfactant, wherein the selected amounts do not collectively exceed 100% w/w.

In particular embodiments, lipid particles disclosed herein include 41-49% w/w cannabinoid, 15-40% w/w olive oil or sesame oil, and 10-25% w/w surfactant, wherein the selected amounts do not collectively exceed 100% w/w.

In particular embodiments, lipid particles disclosed herein include 42-48% w/w cannabinoid, 16-39% w/w olive oil or sesame oil, and 12-24% w/w surfactant, wherein the selected amounts do not collectively exceed 100% w/w.

In particular embodiments, lipid particles disclosed herein include 42-43% (e.g., 42.6%) w/w CBD isolate, 33-35% (e.g., 34%) w/w olive oil or sesame oil, 14-16% (e.g., 15%) w/w sorbitan monooleate, and 8-9% (e.g., 8.4%) w/w polysorbate 80 wherein the selected amounts do not collectively exceed 100% w/w.

In particular embodiments, lipid particles disclosed herein include 35-40% w/w CBD isolate, 28-32% w/w olive oil or sesame oil, 17-22% w/w sorbitan monooleate, and 8-13% w/w polysorbate 80 wherein the selected amounts do not collectively exceed 100% w/w.

In particular embodiments, lipid particles disclosed herein include 48-49% (e.g., 48.2%) w/w CBD isolate, 38-39% (e.g., 38.5%) w/w olive oil or sesame oil, 8-9% (e.g., 8.5%) w/w sorbitan monooleate, and 4-5% (e.g., 4.8%) w/w polysorbate 80 wherein the selected amounts do not collectively exceed 100% w/w.

In particular embodiments, lipid particles disclosed herein include 42-43% (e.g., 42.6%) w/w CBD isolate, 16-18% (e.g., 17%) w/w olive oil or sesame oil, 16-18% (e.g., 17%) w/w limonene, 14-16% (e.g., 15%) w/w sorbitan monooleate, and 8-9% (e.g., 8.4%) w/w polysorbate 80 wherein the selected amounts do not collectively exceed 100% w/w.

In particular embodiments, lipid particles disclosed herein include 35-40% w/w CBD isolate, 13-17% w/w olive oil or sesame oil, 13-17% w/w limonene, 17-22% w/w sorbitan monooleate, and 8-13% w/w polysorbate 80 wherein the selected amounts do not collectively exceed 100% w/w.

In particular embodiments, lipid particles disclosed herein include 42-43% (e.g., 42.6%) w/w CBD isolate, 16-18% (e.g., 17%) w/w olive oil, 17% w/w bisabolol, 15% w/w sorbitan monooleate, and 8.4% w/w polysorbate 80 wherein the selected amounts do not collectively exceed 100% w/w.

In particular embodiments, lipid particles disclosed herein include 42-43% (e.g., 42.6%) w/w CBD isolate, 33-35% (e.g., 34%) w/w bisabolol, 14-16% (e.g., 15%) w/w sorbitan monooleate, and 1-2% (e.g., 1.98%) w/w polysorbate 80 wherein the selected amounts do not collectively exceed 100% w/w.

In particular embodiments, lipid particles disclosed herein include 38-39% (e.g., 38.5%) w/w CBD isolate, 30-31% (e.g., 30.8%) w/w olive oil or sesame oil, 19-20% (e.g., 19.7%) w/w sorbitan monooleate, and 11-12% (e.g., 11.1%) w/w polysorbate 80 wherein the selected amounts do not collectively exceed 100% w/w.

In particular embodiments, lipid particles disclosed herein include 38-39% (e.g., 38.5%) w/w CBD isolate, 15-16% (e.g., 15.4%) w/w olive oil or sesame oil, 15-16% (e.g., 15.4%) w/w limonene, 19-20% (e.g., 19.7) w/w sorbitan monooleate, and 11-12% (e.g. 11.1%) w/w polysorbate 80 wherein the selected amounts do not collectively exceed 100% w/w.

In particular embodiments, lipid particles disclosed herein include 57-70% (e.g., 62%) w/w of a first LC, 0.5-0.9% (e.g., 0.7%) w/w of a second LC, 4-9% (e.g., 7%) w/w surfactant, and 16-21% (e.g., 19%) w/w glycoside, wherein the selected amounts do not collectively exceed 100% w/w.

In particular embodiments, lipid particles disclosed herein include 57-70% (e.g., 62%) w/w of a first LC, 0.5-0.9% (e.g., 0.7%) w/w of a second LC, 4-9% (e.g., 7%) w/w surfactant, 7-10% (e.g., 19%) w/w of a first glycoside, and 9-12% (e.g., 11%) w/w of a second glycoside, wherein the selected amounts do not collectively exceed 100% w/w.

In particular embodiments, lipid particles disclosed herein include 75-63% (e.g., 68%) w/w cannabidiol, 0.5-1% (e.g., 0.8%) w/w tocopherol, 5-10% (e.g., 8%) w/w saponin, 7.5-11% (e.g., 9%) w/w rebaudioside A, and 10-13% (e.g., 12%) w/w mogroside V, wherein the selected amounts do not collectively exceed 100% w/w.

In particular embodiments, lipid particles disclosed herein include 57-70% (e.g., 62%) w/w cannabidiol, 0.5-1% (e.g., 0.7%) w/w tocopherol, 4.5-9% (e.g., 7%) w/w saponin, 7-10% (e.g., 8%) w/w rebaudioside A, and 9-11% (e.g., 11%) w/w mogroside V, wherein the selected amounts do not collectively exceed 100% w/w.

In particular embodiments, lipid particles disclosed herein include 31-29% (e.g., 30%) w/w cannabidiol, 54-43% (e.g., 48%) w/w all-rac-alpha-tocopherol, 1-5% (e.g., 2%) w/w rebaudioside A, 1-5% (e.g., 2.5%) w/w mogroside V, 3-8% (e.g., 6%) w/w polysorbate 80, and 7-12% (e.g., 11%) w/w sorbitane monooleate, wherein the selected amounts do not collectively exceed 100% w/w.

Particles disclosed herein additionally have a high LC to surfactant ratio. For example, in certain particles, the LC to surfactant ratio is at least 2:1. For example, in certain particles, the LC to surfactant ratio is at least 2:1. In particular embodiments, lipid particles disclosed herein include a ratio of an LC to a surfactant of 10:1, 20:3, 5:1, 20:5, 40:11 10:3, 20:7, 5:2, 9:4, 20:9, 2:1, 20:11, 5:3, 20:13, 20:12, 20:13, 20:14, 20:15, 10:7, 20:15, 5:4, 20:17, 10:9, or 1:1 (% w/w). In particular embodiments, the LC can include a first LC and a second LC. In particular embodiments, the first LC to surfactant ratio is at least 2:1. For example, in certain particles, the first LC to surfactant ratio is at least 1:1.

In particular embodiments, lipid particles disclosed herein include a ratio of a first LC and a second LC to a surfactant of 18:1, 9:1, 18:3, 72:11, 9:2, 18:5, 36:11, 6:2, 18:7, 9:4, 6:3, 2:1, 10:8, 14:8, 28:11, 9:5, or 1:1 (% w/w).

In particular embodiments, lipid particles disclosed herein include a ratio of a first LC to a second LC to a surfactant of 5:4:2, 20:16:11, 10:8:5, 10:4:5 10:8:7, 5:4:4, 20:12:11, 5:2:4, 40:32:11, or 10:4:7 (% w/w).

In particular embodiments, lipid particles disclosed herein include a ratio of a first LC to an additional LC of 1:1, 1:2, 1:3, 1:4, 1:5, 1:6, 1:7, 1:8, 1:9, 1:10, 10:1, 9:1, 8:1, 7:1, 6:1, 5:1, 4:1, 3:1, 2:1, 5:4, 3:8, 9:2, 2:3, or 1:1 (% w/w).

As used herein, particles refer to small localized objects having a density, volume, and mass.

Lipid particles refer to particles including an LC. As indicated previously, LC refers to a compound that has the ability to dissolve in lipids and non-polar solvents. LCs have an affinity for lipids. LCs generally include oils, fatty esters, hydrocarbon oils, silicones, waxes, fatty alcohols, lipophilic vitamins, and phospholipids. Within the context of the current disclosure, hydrophobic fluorocarbons are considered LCs.

In particular embodiments, the present disclosure includes an LC that is naturally-occurring in plants. An LC that is naturally-occurring in plants means that the LC can be found in a plant but does mean that the LC originates from any plant. For example, the LC can be extracted from the plant or the LC can be synthetically produced. In other embodiments, LCs can be artificial meaning that they are compounds that can be synthesized but do not otherwise exist in the natural environment.

In certain other examples, the current disclosure provides lipid particles including (i) at least two LCs and (ii) a surfactant, wherein at least one LC is solid at room temperature, and at least a percentage of the surfactant is on the surface of the lipid particle. Within these particles, the two LCs can be derived from a common plant type (e.g., cannabis) to prepare a particle with a high concentration of components from the common plant type.

Examples of lipid particles include solid lipid particles, liposomes, and exosomes. A solid lipid particle (SLP) refers to lipid components wherein at least one lipid component is solid at temperatures of at least 50° C. SLPs can also be defined including a lipid matrix that is solid at room and body temperatures that is stabilized by the presence of a surfactant. For additional information on particle types, including lipid particle types, see Yetisgin et al., Molecules 2020, 25, 2193.

In certain examples, the SLP can be a solid lipid microparticle or a solid lipid nanoparticle. A microparticle refers to any particle having a diameter ranging from 1 to 1000 μm. A nanoparticle refers to any particle having a diameter less than 1000 nm. Particle diameter can be measured by light scattering instruments, such as those offered for sale by Malvern Panalytical (e.g., the Mastersizer 3000) or Brookhaven Instruments (e.g., the NanoBrook 90Plus PALS) at specified operation conditions.

In certain examples, the current disclosure provides an aqueous dispersion or composition including lipid particles as described herein. As used herein, an aqueous dispersion refers to a mixture in which at least one discontinuous phase, often in the form of one or more kinds of particles, is dispersed in an aqueous media. For example, in certain instances an aqueous dispersion refers to a liquid system in which particles are capable of being substantially uniformly dispersed. In certain examples, after agitation, particles within an aqueous dispersion remain substantially uniformly dispersed, without further agitation for a specified time period, such as at least 30 minutes, at least 1 hour, at least 3 hours, at least 24 hours, at least 48 hours, at least 1 week, or at least 1 month. Within certain examples described herein, aqueous refers to any liquid composition in which the majority of the liquid component is water. In certain examples, aqueous refers to any liquid composition in which the liquid component is pure water. Other examples include liquid compositions in which the liquid component is at least 80% water, at least 85% water, at least 90% water, at least 95% water, or at least 98% water, or at least 99% water. A liquid composition can include a liquid carrier and lipid particles disclosed herein but need not be an aqueous dispersion. In certain examples, the liquid composition can be aqueous or non-aqueous. In certain examples, the liquid composition is aqueous. As indicated elsewhere herein, a dry composition has less than 10% moisture.

Like particles, aqueous dispersions and compositions disclosed herein have high LC concentrations and high LC to surfactant ratios. Compositions can be aqueous compositions, liquid compositions, or dry compositions.

In particular embodiments, an aqueous dispersion or liquid composition includes 1-99% w/w first LC, 1-99% w/w second LC, 0.5-10% w/w surfactant, and water, wherein the selected amounts do not collectively exceed 100% w/w.

In particular embodiments, an aqueous dispersion or liquid composition includes 2-99% w/w first LC and second LC, 0.5-10% w/w surfactant, and water, wherein the selected amounts do not collectively exceed 100% w/w.

In particular embodiments, an aqueous dispersion or liquid composition includes at least 10% w/w first LC, a % w/w amount of a second LC that is less than the amount of the first LC, a % w/w amount of a surfactant that is less than half of the % w/w of the first LC, and water, wherein the selected amounts do not collectively exceed 100% w/w.

In particular embodiments, an aqueous dispersion or liquid composition includes at least 8% w/w first LC, 0.5%-10% w/w surfactant, with a remainder of water, wherein the selected amounts do not collectively exceed 100% w/w.

In particular embodiments, an aqueous dispersion or liquid composition includes at least 8% w/w first LC, 2-12% w/w second LC, and 0.5%-10% w/w surfactant, with a remainder of water, wherein the selected amounts do not collectively exceed 100% w/w.

In particular embodiments, an aqueous dispersion or liquid composition includes at least 10-20% w/w first and second LCs, and 0.5%-10% w/w surfactant, with a remainder of water, wherein the selected amounts do not collectively exceed 100% w/w.

In particular embodiments, an aqueous dispersion or liquid composition includes 10% w/w first LC, 8% w/w second LC, 5.5% w/w surfactant, and 76.5% w/w water.

In particular embodiments, an aqueous dispersion or liquid composition includes 10% w/w first LC, 8% w/w second LC, 8% w/w surfactant, and 74% w/w water.

In particular embodiments, an aqueous dispersion or liquid composition includes 10% w/w first LC, 8% w/w second LC, 2.75% w/w surfactant, and 79.25% w/w water.

In particular embodiments, an aqueous dispersion or liquid composition includes 10% w/w first LC, 4% w/w second LC, 4% w/w third LC, 5.5% w/w surfactant, and 76.5% w/w water.

In particular embodiments, an aqueous dispersion or liquid composition includes 10% w/w first LC, 4% w/w second LC, 4% w/w third LC, 8% w/w surfactant, and 74% w/w water.

In particular embodiments, an aqueous dispersion or liquid composition includes 4.8-5.7% w/w a first LC, 0-0.1% w/w a second LC, 0.3-1% w/w surfactant, 1.1-2.1% w/w glycoside, and 90-94% w/w water.

In particular embodiments, an aqueous dispersion or liquid composition includes 10% w/w CBD isolate, 8% w/w olive oil, 3.52% w/w sorbitan monooleate, 1.98% w/w polysorbate 80, and 76.5% w/w water.

In particular embodiments, an aqueous dispersion or liquid composition includes 10% w/w CBD isolate, 8% w/w olive oil, 5.12% w/w sorbitan monooleate, 2.88% w/w polysorbate 80, and 74% w/w water.

In particular embodiments, an aqueous dispersion or liquid composition includes 10% w/w CBD isolate, 8% w/w olive oil, 1.76% w/w sorbitan monooleate, 0.99% w/w polysorbate 80, and 79.25% w/w water.

In particular embodiments, an aqueous dispersion or liquid composition includes 10% w/w CBD isolate, 4% w/w olive oil, 4% w/w limonene, 3.52% w/w sorbitan monooleate, 1.98% w/w polysorbate 80, and 76.5% w/w water.

In particular embodiments, an aqueous dispersion or liquid composition includes 10% w/w CBD isolate, 4% w/w olive oil, 4% w/w limonene, 5.12% w/w sorbitan monooleate, 2.88% w/w polysorbate 80, and 74% w/w water.

In particular embodiments, an aqueous dispersion or liquid composition includes 10% w/w CBD isolate, 4% w/w olive oil, 4% w/w bisabolol, 3.52% w/w sorbitan monooleate, 1.98% w/w polysorbate 80, and 76.5% w/w water.

In particular embodiments, an aqueous dispersion or liquid composition includes 10% w/w CBD isolate, 8% w/w bisabolol, 3.52% w/w sorbitan monooleate, 1.98% w/w polysorbate 80, and 76.5% w/w water.

In particular embodiments, an aqueous dispersion or liquid composition includes 4.8-5.8% w/w cannabidiol, 0-0.1% w/w tocopherol, 0.3-1% w/w saponin, 0.5-1% w/w rebaudioside A, 0.6-1.1% w/w mogroside V, and 90-94% w/w water.

In particular embodiments, an aqueous dispersion or liquid composition includes 8.8-11% w/w cannabidiol, 15-16% w/w all-rac-alpha-tocopherol, 0.3-2% w/w rebaudioside A, 0.3-2% w/w mogroside V, 1-2.7% w/w polysorbate 80, 2.2-4.5% w/w sorbitan monooleate, and 62-72% w/w water.

In particular embodiments, the ratio of LCs to surfactant within the aqueous dispersion or composition can be at least 2:1. In particular embodiments, the ratio of a first LC (e.g., CBD) to surfactant within the aqueous dispersion or composition can be at least 2:1. In particular embodiments, the ratio of LCs to surfactant within the aqueous dispersion or composition can be at least 1:1. In particular embodiments, the ratio of a first LC (e.g., CBD) to surfactant within the aqueous dispersion or composition can be at least 1:1.

In particular embodiments, lipid particles disclosed herein include a ratio of LC to glycoside of at least 1:1, 1.5:1, 2:1, 5:1, 6:1, 7:1, 8:1, 9:1, 10:1 (w/w).

In certain examples, an aqueous dispersion or composition includes lipid particles within a liquid wherein the lipid particles include (i) a first LC and (ii) a surfactant, wherein the ratio of the first LC to the surfactant within the aqueous dispersion or composition is at least 2:1 (% w/w). In certain examples, an aqueous dispersion or composition includes lipid particles within a liquid wherein the lipid particles include (i) a first LC and (ii) a surfactant, wherein the ratio of the first LC to the surfactant within the aqueous dispersion or composition is at least 1:1 (% w/w).

In certain examples, an aqueous dispersion or composition includes lipid particles within a liquid wherein the lipid particles include (i) a first LC, (ii) a second LC, and (iii) a surfactant, wherein the ratio of the first LC to the surfactant within the aqueous dispersion or composition is at least 2:1 (% w/w). In certain examples, an aqueous dispersion or composition includes lipid particles within a liquid wherein the lipid particles include (i) a first LC, (ii) a second LC, and (iii) a surfactant, wherein the ratio of the first LC to the surfactant within the aqueous dispersion or composition is at least 1:1 (% w/w).

In certain examples, an aqueous dispersion or composition includes lipid particles within a liquid wherein the lipid particles include (i) a first LC, (ii) a second LC, and (iii) a surfactant, wherein the ratio of the first and second LCs to the surfactant within the aqueous dispersion or composition is at least 2:1 (% w/w). In certain examples, an aqueous dispersion or composition includes lipid particles within a liquid wherein the lipid particles include (i) a first LC, (ii) a second LC, and (iii) a surfactant, wherein the ratio of the first and second LCs to the surfactant within the aqueous dispersion or composition is at least 1:1 (% w/w).

In particular embodiments, an aqueous dispersion or composition includes a ratio of a first LC to surfactant of 10:1, 20:3, 5:1, 20:5, 40:11 10:3, 20:7, 5:2, 9:4, 20:9, 2:1, 20:11, 5:3, 20:13, 20:12, 20:13, 20:14, 20:15, 10:7, 20:15, 5:4, 20:17, 10:9, or 1:1 (% w/w).

In particular embodiments, an aqueous dispersion or composition includes a ratio of a first LC and a second LC to a surfactant of 18:1, 9:1, 18:3, 72:11, 9:2, 18:5, 36:11, 6:2, 18:7, 9:4, 6:3, 2:1, 10:8, 14:8, 28:11, 9:5, or 1:1 (% w/w).

In particular embodiments, an aqueous dispersion or composition includes a ratio of a first LC to a second LC to a surfactant of 5:4:2, 20:16:11, 10:8:5, 10:4:5 10:8:7, 5:4:4, 20:12:11, 5:2:4, 40:32:11, or 10:4:7 (% w/w).

In particular embodiments, an aqueous dispersion or composition includes a ratio of a first LC to an additional LC of 1:1, 1:2, 1:3, 1:4, 1:5, 1:6, 1:7, 1:8, 1:9, 1:10, 10:1, 9:1, 8:1, 7:1, 6:1, 5:1, 4:1, 3:1, 2:1, 5:4, 3:8, 9:2, 2:3, or 1:1 (% w/w).

In particular embodiments, the current disclosure provides an aqueous dispersion or liquid composition including (i) lipid particles including a first LC, at least one additional LC, and a surfactant, and (ii) an aqueous media, wherein the aqueous dispersion or liquid composition includes over 100 mg of the first LC in 1 mL of the aqueous media.

In particular embodiments, the first LC is a cannabinoid, the second or at least one additional LC is olive oil, sesame oil, limonene, and/or bisabolol, and the liquid or aqueous media is water.

Particles, aqueous dispersion and liquid compositions can be formulated into a variety of delivery vehicles for a variety of purposes. For example, particles, aqueous dispersions, and compositions can be prepared as or within beverages, beverage kits, food products, consumer products, and formulations (e.g., oral formulations, mucosal formulations, ocular formulations, topical formulations, and pharmaceutical formulations).

In particular embodiments, the current disclosure provides a beverage including 0.005-1.0% w/w of an aqueous dispersion disclosed herein, 0.01-0.5% w/w of lemon juice concentrate, 0.01-0.5% w/w of rooibos, 0.01-0.5% w/w of peppermint, 0.01-0.5% w/w of ashwaganda, 0.1-0.9% w/w of tangerine essence, 0.2-5.0% w/w of jasmine green tea, 0.5-5.0% w/w of apple juice concentrate, 0.5-5.0% w/w of tangerine concentrate, and 98.7-81.1% w/w of water wherein the selected amounts do not exceed 100% w/w.

Typically, smaller particles for lipid-in-water emulsions and dispersions lead to a number of occurrences in direct proportion to decreasing particle size: (1) a requirement for more surfactant(s) often in relative ratios of 1:1 to 1:10 lipophilics-to surfactant to generate a stable emulsion or dispersion, (2) decreased palatability due to both the increasing surfactant content and the smaller particles facilitating increased interaction with chemoreceptors causing the particles to taste bitter, and (3) less protection from environmental insults, such as heat, light and oxidation that can lead to the chemical degradation of the active ingredient(s) of the emulsion or dispersion. As described herein, it was discovered that the particles described herein did not exhibit the same relationship for (2) and (3) above as a result of decreasing particle size. Furthermore, it was unexpectedly determined that the particles of the instant disclosure require relative ratios of at least 2:1 lipophilics-to-surfactant, which is well below the amount of surfactant required by typical emulsions and liquid dispersions.

Formulations, as used herein, refers to an aqueous dispersion, an aqueous formulation, a liquid composition, or a dry composition unless a stated moisture content requires that the embodiment be classified as aqueous and/or liquid, or dry.

Within certain examples described herein, aqueous formulations refer to any liquid composition in which the majority of the liquid component is water. In certain examples, aqueous formulations refer to any liquid composition in which the liquid component is pure water. Other examples include liquid compositions in which the liquid component is at least 80% water, at least 85% water, at least 90% water, at least 95% water, or at least 98% water, or at least 99% water. A liquid composition that is not aqueous refers to any liquid composition in which the majority of the liquid component is not water.

Aqueous formulations and liquid compositions described herein can take the form of, for example, solutions, emulsions, syrups, concentrations, or suspensions, or they can be presented as a dry composition for constitution with water or other suitable vehicle before use or as ingestible solid oral dosage forms such as pills, tablets, mucoadhesive tablets or capsules. Aqueous formulations can include from 10% to 99.99% moisture. In particular embodiments, the aqueous formulation may include a moisture content from 10% to 99%, 10% to 70%, 20% to 60%, 30% to 50%, 20% to 40%, 10 to 30%, 50% to 99%, 50% to 80%, 60% to 70%, 70% to 99%, 80% to 99%, or 90% to 99.99%. Within this aspect of the disclosure 10% is a precise value and should not be interpreted as preceded by the word “about”.

A dry composition may for example, be formulated as a powder, a granular material, a crystalline material, other types of dry particle preparations or combinations thereof. In some cases, dry compositions may include ingestible solid oral dosage forms such as pills, tablets, mucoadhesive tablets or capsules. The dry composition includes less than 10% moisture. The dry composition may be formulated by mixing dry ingredients without the addition of a liquid. In one aspect, the dry composition may be prepared by mixing the dry ingredients and liquid ingredients, as disclosed herein, to form a solution, emulsion, syrup, concentrate, or suspension, and then drying the solution, emulsion, syrup, concentrate, or suspension to a dry powder form by conventional drying methods. Representative drying methods may include for example, lyophilization (or freeze drying), spray drying, fluid bed drying, drum drying, pulse combustion drying and various combinations thereof. In one aspect, the method is a spray drying method. The dried composition may include a moisture content from 1% to 9%, 1% to 6%, 2% to 5%, 3% to 4%, 0.01% to 3%, 2% to 3%, 3% to 6%, 4% to 5%, 4% to 9%, 5% to 9%, 6% to 8%, 7% to 9%, or 8% to 9%. The current disclosure provides aqueous and dry composition having high LC concentrations and high LC to surfactant ratios. The aqueous and dry compositions can be used to efficiently deliver active compounds and/or active ingredients, such as cannabinoids.

In particular embodiments, a formulation including a particle of the disclosure and a formulation including a glycoside and a surfactant of the disclosure are prepared separately and later mixed.

Aspects of the current disclosure are now described with additional detail and options as follows: (i) LCs, (ii) Surfactants, (iii) Glycosides, (iv) Formulations, (v) Beverages or Beverage Kits, (vi) Food Products, (vii) Oral Formulations, (viii) Mucosal Formulations, (ix) Consumer Products, (x) Common Considerations & Components for Drinkable, Ingestible, and/or Inhalable Products & Formulations, (xi) Ocular Formulations, (xii) Topical Formulations, (xiii) Pharmaceutical Formulations, (xiv) Common Considerations for all Products and Formulations, (xv) Administration, (xvi) Exemplary Embodiments, (xvii) Experimental Examples, and (xviii) Closing Paragraphs. While section headings are provided, these headings are for organizational purposes only and do not limit the scope or interpretation of the disclosure with the following caveat: surfactants are selected from section (ii), and when present, glycosides are selected from section (iii). If the selected surfactant is saponin, the glycoside will not also be saponin.

(i) LCs. Particles, aqueous dispersions, and compositions disclosed herein include an LC. In certain examples, LCs can be derived from plants. Many families of plants exist, each including several species of plants. Exemplary species within plant families are provided below, and lipophilic components can be derived from each of them. As used herein, “derived from” means either extracted from or synthetically produced.

Acanthaceae: Acanthus mollis; Justicia adhatoda.

Actinidiaceae: Actinidia chinensis; Actinidia deliciosa; Actinidia sp.

Adoxaceae: Sambucus canadensis; Sambucus chinensis; Sambucus edulus; Sambucus nigra; Sambucus sieboldiana; Sambucus sp.; Viburnum lantana; Viburnum opulus; Viburnum rhytidophyllum; Viburnum sp.; Viburnum tinus.

Aizoaceae: Mesembryanthemum crystallinum.

Alstroemeriaceae: Alstroemeria sp.

Amaranthaceae: Alternanthera sp.; Amaranthus blitum; Amaranthus caudatus; Amaranthus graecizans; Amaranthus hybridus; Amaranthus mangostanus; Amaranthus palmeri; Amaranthus retroflexus; Amaranthus sp.; Amaranthus spinosus; Amaranthus viridis; Atriplex canescens; Atriplex lentiformis; Atriplex semibaccata; Beta vulgaris; Celosia argentea; Chenopodium album; Chenopodium murale; Chenopodium sp.; Dysphania ambrosioides; Haloxylon ammodendron; Iresine herbstii; Salsola vermiculata; Spinacia oleracea.

Amaryllidaceae: Allium ampeloprasum; Allium cepa; Allium fistulosum; Allium sativum; Allium sp.; Narcissus sp.

Anacardiaceae: Mangifera indica; Pistacia terebinthus; Pistacia vera.

Annonaceae: Annona muricata; Annona reticulata; Annona squamosa.

Apiaceae: Aegopodium podagraria; Ammi majus; Apium graveolens; Apium nodiflorum; Arracacia xanthorrhiza; Athamanta macedonica; Bupleurum lancifolium; Coriandrum sativum; Cryptotaenia canadensis; Daucus carota; Eryngium sp.; Foeniculum vulgare; Pastinaca sativa; Petroselinum crispum; Peucedanum japonicum; Phellolophium madagascariense; Spananthe sp.

Apocynaceae: Ampelamus laevis; Apocynum cannabinum; Asclepias sp.; Catharanthus roseus; Mandevilla sp.; Matelea carolinensis; Nerium oleander; Plumeria sp.; Raphionacme sp.; Rauvolfia serpentina; Vinca major; Vinca sp.

Aquifoliaceae: Ilex crenata.

Araceae: Alocasia macrorrhizos; Alocasia sp.; Anthurium sp.; Arum italicum; Arum sp.; Caladium bicolor; Caladium sp.; Calla sp.; Colocasia esculenta; Co/ocasia sp.; Dieffenbachia sp.; Epipremnum pinnatum; Philodendron sp.; Symplocarpus foetidus; Xanthosoma sp.; Zantedeschia aethiopica.

Araliaceae: Aralia sp.; Hedera canariensis; Hedera helix; Hedera sp.; Hydrocotyle umbellata; Polyscias balfouriana; Schejflera actinophylla; Schejflera elegantissima; Schejflera sp.; Tetrapanax papyrifer.

Araucariaceae: Agathis sp.; Araucaria sp.

Arecaceae: Dypsis sp.; Phoenix dactylifera; Phoenix sp.; Veitchia sp.

Aristolochiaceae: Aristolochia clematitis.

Asparagaceae: Asparagus laricinus; Asparagus officinalis; Asparagus setaceus; Asparagus sp.; Aspidistra elatior; Cordyline fruticosa; Cordyline sp.; Dracaena braunii; Dracaena fragrans; Dracaena goldieana; Dracaena sp.; Hyacinthus orientalis; Eachenalia ensifolia; Maianthemum racemosum; Ornithogalum sp.; Polygonatum odoratum; Ruscus aculeatus; Yucca sp.

Balsaminaceae: Impatiens balsamina; Impatiens sp.; Impatiens walleriana.

Berberidaceae: Berberis cretica; Berberis thunbergii; Berberis vulgaris; Berberis wilsoniae; Nandina domestica.

Betulaceae: Alnus incana; Betula maximowicziana; Betula papyrifera; Betula pendula; Carpinus sp.; Corylus avellana.

Bignoniaceae: Campsis radicans; Pyrostegia venusta; Tecoma capensis; Tecoma stans. Boraginaceae: Borago officinalis; Cynoglossum columnae; Heliotropium arborescens; Heliotropium eichwaldii; Heliotropium europaeum; Nama hispidum; Omphalodes verna.

Brassicaceae: Aethionema saxatile; Brassica juncea; Brassica napus; Brassica oleracea; Brassica rapa; Brassica sp.; Capsella bursa-pastoris; Diplotaxis erucoides; Diplotaxis viminea; Eruca vesicaria; Erysimum graecum; Erysimum sp.; Erysimum x cheiri; Hirschfeldia incana; Eepidium didymum; Malcolmia sp.; Matthiola fruticulosa; Matthiola incana; Matthiola odoratissima; Nasturtium sp.; Raphanus raphanistrum; Raphanus sp.; Rapistrum rugosum; Rorippa indica; Sinapis arvensis; Zilla spinosa.

Bromeliaceae: Tillandsia sp.

Buxaceae: Buxus sempervirens.

Calophyllaceae: Mammea Americana.

Campanulaceae: Campanula erinus; Lobelia sp.; Platycodon grandiflorus.

Cannabaceae: Cannabis sativa; Cannabis indica; Cannabis ruderalis; Celtis australis; Celtis occidentalis; Humulus lupulus; americanus; Humulus cordifolius; Humulus neomexicanus; Humulus pubescens; Humulus scandens; Trema micrantha.

Cannaceae: Canna indica.

Capparaceae: Capparis nummularia.

Caprifoliaceae: Cephalaria gigantea; Diervilla sp.; Leycesteria formosa; Lonicera etrusca; Lonicera nigra; Lonicera periclymenum; Lonicera sp.; Lonicera tatarica; Lonicera xylosteum; Pterocephalus plumosus; Scabiosa sicula; Symphoria racemosa; Symphoricarpos albus; Symphoricarpos orbiculatus; Weigela hortensis.

Caricaceae: Carica papaya.

Caryophyllaceae: Dianthus armeria; Dianthus barbatus; Dianthus caryophyllus; Dianthus chinensis; Dianthus sp.; Dianthus tenuiflorus; Drymaria cordata; Gypsophila paniculata; Myosoton aquaticum; Silene chalcedonica; Silene vulgaris; Stellaria media.

Celastraceae: Celastrus orbiculatus; Celastrus scandens; Euonymus europaeus; Euonymus japonicus; Euonymus sp.

Cistaceae: Helianthemum salicifolium. Cleomaceae: Cleome sp.; Cleome viscosa.

Clethraceae: Clethra arborea.

Combretaceae: Terminalia catappa.

Commelinaceae: Commelina benghalensis; Commelina communis; Commelina diffusa.

Compositae: Acanthospermum hispidum; Achillea filipendulina; Achillea fraasii; Ageratum conyzoides; Ageratum houstonianum; Ambrosia trifida; Anthemis chia; Arctium lappa; Arctium minus; Arctotheca calendula; Arctotis sp.; Artemisia dracunculus; Beilis annua; Bidens bipinnata; Bidens biternata; Bidens pilosa; Bidens sp.; Boltonia sp.; Brachyscome sp.; Calendula arvensis; Calendula officinalis; Calendula sp.; Callistephus chinensis; Carduus crispus; Carthamus tinctorius; Centaurea cyanus; Centaurea hyalolepis; Centaurea iberica; Centaurea imperialis; Centaurea montana; Chaenactis stevioides; Chrysanthemum coronarium; Chrysanthemum indicum; Chrysanthemum morifolium; Chrysanthemum segetum; Chrysanthemum sp.; Chrysothamnus viscidiflorus; Cichorium endivia; Cichorium intybus; Cichorium pumilum; Cichorium spinosum; Conyza bonariensis; Conyza canadensis; Conyza sp.; Cosmos bipinnatus; Cosmos sp.; Crassocephalum crepidioides; Crepis neglecta; Crepis rubra; Cynara cardunculus; Cynara sp.; Dahlia coccinea; Dahlia sp.; Dahlia variabilis; Elephantopus mollis; Erigeron annuus; Erigeron sp.; Euryops sp.; Euthamia graminifolia; Galinsoga caracasana; Galinsoga ciliata; Galinsoga parviflora; Gerbera jamesonii; Gerbera sp.; Helianthella quinquenervis; Helianthus annuus; Helichrysum luteoalbum; Helichrysum tenax; Helichrysum thianschanicum; Heliopsis sp.; Helminthotheca echioides; Lactuca saligna; Lactuca sativa; Lactuca serriola; Eapsana communis; Eeontodon autumnalis; Eeucanthemum vulgare; Melampodium perfoliatum; Melanthera aspera; Mikania micrantha; Montanoa bipinnatifida; Notobasis syriaca; Osteospermum sp.; Parthenium sp.; Pentzia globosa; Picris pauciflora; Picris sprengeriana; Pseudo gnaphalium obtusifolium; Rudbeckia amplexicaulis; Rudbeckia sp.; Schkuhria pinnata; Scolymus maculatus; Scorzonera sp.; Senecio lividus; Senecio sp.; Senecio vulgaris; Solidago gigantea; Sonchus arvensis; Sonchus asper; Sonchus oleraceus; Sonchus sp.; Tagetes erecta; Tagetes microglossa; Tagetes minuta; Tagetes patula; Tagetes sp.; Taraxacum officinale; Tithonia rotundifolia; Tragopogon dubius; Tragopogon pratensis; Tridax procumbens; Urospermum dalechampii; Vernonia sp.; Xanthium strumarium; Zinnia elegans; Zinnia sp.

Convolvulaceae: Calystegia hederacea; Calystegia sepium; Convolvulaceae sp.; Convolvulus arvensis; Convolvulus hirsutus; Convolvulus scammonia; Convolvulus siculus; Convolvulus sp.; Convolvulus tricolor; Dinetus racemosus; Ipomoea aquatica; Ipomoea arachnosperma; Ipomoea batatas; Ipomoea biflora; Ipomoea cairica; Ipomoea hochstetteri; Ipomoea indica; Ipomoea lacunosa; Ipomoea lobata; Ipomoea nil; Ipomoea purpurea; Ipomoea sp.; Ipomoea tricolor; Ipomoea triloba.

Cornaceae: Cornus alba; Cornus canadensis; Cornus nuttallii; Cornus sp.

Cucurbitaceae: Benincasa hispida; Bryonia alba; Citrullus colocynthis; Citrullus lanatus; Cucumis melo; Cucumis sativus; Cucumis sp.; Cucurbita ficifolia; Cucurbita maxima; Cucurbita moschata; Cucurbita pepo; Cucurbita sp.; Cucurbitaceae sp.; Diplocyclos palmatus; Ecballium elaterium; Lagenaria siceraria; Luffa acutangula; Luff a cylindrica; Momordica charantia; Praecitrullus fistulosus; Sechium edule.

Cupressaceae: Chamaecyparis thyoides; Cupressus sp.; Juniperus arizonica; Juniperus virginiana; Platycladus orientalis.

Cyperaceae: Cyperus esculentus; Cyperus rotundus; Cyperus schimperianus.

Dipterocarpaceae: Shorea robusta.

Ebenaceae: Diospyros kaki; Diospyros scabrida.

Elaeagnaceae: Elaeagnus angustifolia; Elaeagnus umbellata.

Equisetaceae: Equisetum palustre.

Ericaceae: Azalea nudiflora; Azalea sp.; Rhododendron sp.; Siphonandra sp.

Euphorbiaceae: Acalypha australis; Acalypha havanensis; Acalypha sp.; Acalypha wilkesiana; Codiaeum sp.; Codiaeum variegatum; Croton niveus; Croton sp.; Euphorbia amygdaloides; Euphorbia burmanni; Euphorbia helenae; Euphorbia helioscopia; Euphorbia hirta; Euphorbia hypericifolia; Euphorbia parviflora; Euphorbia pulcherrima; Euphorbia sp.; Hevea brasiliensis; Hura crepitans; Jatropha gossypiifolia; Jatropha hastata; Jatropha multifida; Jatropha sp.; Manihot esculenta; Manihot sp.; Mercurialis annua; Mercurialis sp.; Ricinus communis.

Fagaceae: Quercus alba; Quercus robur; Quercus sp. Garryaceae: Aucuba japonica.

Gentianaceae: Eustoma grandiflorum; Gentiana sp.

Geraniaceae: Erodium alnifolium; Geranium carolinianum; Geranium dissectum; Geranium lucidum; Geranium molle; Geranium rotundifolium; Geranium sp.; Pelargonium inquinans; Pelargonium sp.

Gesneriaceae: Saintpaulia ionantha.

Goodeniaceae: Goodenia sp.; Scaevola sp.

Heliconiaceae: Heliconia bihai; Heliconia latispatha.

Hydrangeaceae: Deutzia sp.; Hydrangea macrophylla; Hydrangea paniculata; Hydrangea sp.; Philadelphus coronarius; Philadelphus sericanthus.

Iridaceae: Crocosmia x crocosmiiflora; Gladiolus hortulanus; Gladiolus italicus; Gladiolus sp.; Iris sanguine a; Iris x germanica; Ixiaflexuosa.

Juglandaceae: Carya illinoinensis; Juglans regia; Juglans sp.

Lamiaceae: Ajuga sp.; Ballota africana; Clerodendrum chinense; Clerodendrum thomsoniae; Galeopsis speciosa; Galeopsis tetrahit; Glechoma hederacea; Glechoma sp.; Holmskioldia sanguine a; Holmskioldia sp.; Eamium album; Lamium amplexicaule; Eamium purpureum; Eamium sp.; Lavandula sp.; Leonotis ocymifolia; Leucas martinicensis; Marrubium vulgare; Melissa officinalis; Mentha arvensis; Mentha sp.; Mentha spicata; Mentha x piperita; Moluccella laevis; Monarda fistulosa; Nepeta cataria; Ocimum basilicum; Ocimum tenuiflorum; Perilla frutescens; Rosmarinus officinalis; Salvia argentea; Salvia officinalis; Salvia pratensis; Salvia sp.; Salvia splendens; Salvia verticillata; Salvia viridis; Stachys arvensis; Vitex negundo.

Lauraceae: Cassytha sp.; Endlicheria paniculata; Laurus nobilis; Persea americana.

Leguminosae: Acacia greggii; Acacia horrida; Acacia huarango; Acacia karroo; Acacia robusta; Acacia sp.; Alysicarpus longifolius; Amphicarpaea bracteata; Anthyllis vulneraria; Arachis hypogaea; Arachis sp.; Astragalus sinicus; Bauhinia forficata; Bauhinia monandra; Bauhinia sp.; Bauhinia variegata; Bituminaria bituminosa; Canavalia ensiformis; Caragana arborescens; Cassia artemisioides; Ceratonia siliqua; Cercis siliquastrum; Cicer arietinum; Clianthus sp.; Clitoria ternatea; Coronilla valentina; Crotalaria juncea; Crotalaria micans; Crotalaria sp.; Dalbergia sissoo; Dalea mollis; Desmodium khasianum; Dolichos sp.; Erythrina corallodendron; Erythrina poeppigiana; Erythrina sp.; Genista sp.; Gleditsia sp.; Glycine max; Indigofera arrecta; Indigofera holubii; Indigofera tinctoria; Inga sp.; Kennedia coccinea; Eablab purpureus; Laburnum anagyroides; Laburnum sp.; Lathyrus cicera; Lathyrus odoratus; Lathyrus sativus; Lens culinaris; Lespedeza maximowiczii; Lotus corniculatus; Lupinus arboreus; Lupinus argenteus; Lupinus sativus; Macroptilium atropurpureum; Macroptilium lathyroides; Medicago arabica; Medicago arborea; Medicago lupulina; Medicago orbicularis; Medicago polymorpha; Medicago sativa; Medicago sp.; Melilotus albus; Melilotus indicus; Melilotus sp.; Mucuna membranacea; Mucuna pruriens; Neonotonia wightii; Neorautanenia mitis; Onobrychis viciifolia; Ornithopus sp.; Phaseolus acutifolius; Phaseolus coccineus; Phaseolus lunatus; Phaseolus sp.; Phaseolus vulgaris; Pisum sativum; Psophocarpus tetragonolobus; Pueraria montana; Pueraria phaseoloides; Rhynchosia capitata; Rhynchosia caribaea; Robinia hispida; Robinia pseudoacacia; Sesbania cannabina; Sesbania herbacea; Spartium junceum; Styphnolobium japonicum; Teramnus uncinatus; Tipuana tipu; Trifolium alexandrinum; Trifolium aureum; Trifolium dasyurum; Trifolium dubium; Trifolium glome ratum; Trifolium hybridum; Trifolium incarnatum; Trifolium pratense; Trifolium purpureum; Trifolium repens; Trifolium sp.; Trifolium spumosum; Vicia angustifolia; Vicia faba; Vicia pulchella; Vicia sativa; Vicia sp.; Vicia villosa; Vigna aconitifolia; Vigna angularis; Vigna mungo; Vigna radiata; Vigna sp.; Vigna unguiculata; Wisteria floribunda; Wisteria polystachya; Wisteria sinensis.

Liliaceae: Lilium sp.

Linaceae: Reinwardtia tetragyna.

Lythraceae: Cuphea sp.; Lagerstroemia speciosa; Punica granatum.

Magnoliaceae: Magnolia liliiflora; Magnolia sp.; Magnolia stellata.

Malvaceae: Abelmoschus esculentus; Abutilon pictum; Abutilon reflexum; Abutilon sp.; Abutilon theophrasti; Abutilon tubulosum; Alcea rosea; Althaea nudiflora; Byttneria australis; Ceiba pentandra; Corchorus capsularis; Corchorus olitorius; Gossypium barbadense; Gossypium herbaceum; Gossypium hirsutum; Gossypium sp.; Grewia asiatica; Grewia biloba; Helicteres guazumifolia; Hibiscus lunariifolius; Hibiscus mutabilis; Hibiscus rosa-sinensis; Hibiscus sp.; Hibiscus syriacus; Hibiscus trionum; Malva aegyptia; Malva moschata; Malva neglecta; Malva nicaeensis; Malva parviflora; Malva sp.; Malva sylvestris; Malva trimestris; Malvella leprosa; Sida rhombifolia; Sida sp.; Sterculia murex; Tilia americana; Tilia cordata; Tilia platyphyllos; Tilia rubra; Tilia sp.; Tilia tomentosa; Tilia x euchlora; Triumfetta semitriloba; Waltheria indica.

Marantaceae: Calathea sp.; Maranta sp.

Meliaceae: Azadirachta indica; Melia azedarach; Toona ciliata.

Menispermaceae: Tinospora fragosa.

Moraceae: Artocarpus altilis; Ficus carica; Ficus elastica; Ficus religiosa; Ficus sp.; Morus alba; Morus nigra; Morus rubra; Morus sp.

Moringaceae: Moringa oleifera.

Musaceae: Musa acuminata; Musa basjoo; Musa sp.; Musa x paradisiaca.

Myrtaceae: Eucalyptus grandis; Psidium cattleianum; Psidium guajava; Syzygium cumini.

Nothofagaceae: Nothofagus alpina.

Nyctaginaceae: Bougainvillea spectabilis.

Olacaceae: Ximenia americana.

Oleaceae: Forsythia koreana; Forsythia suspensa; Forsythia x intermedia; Fraxinus angustifolia; Fraxinus excelsior; Fraxinus ornus; Fraxinus sp.; Jasminum humile; Jasminum nudiflorum; Jasminum officinale; Jasminum sambac; Jasminum sp.; Ligustrum lucidum; Ligustrum vulgare; Olea europaea; Osmanthus fragrans; Syringa oblata; Syringa vulgaris.

Onagraceae: Chylismia claviformis; Epilobium angustifolium; Fuchsia magellanica; Fuchsia sp.; Fuchsia x hybrida; Gaura sp.; Oenothera biennis; Oenothera laciniata; Oenothera sp.; Oenothera tetraptera.

Orchidaceae: Catasetum sp.; Cymbidium sp.; Orchidaceae sp.; Papilionanthe teres.

Oxalidaceae: Oxalis corniculata; Oxalis debilis; Oxalis europaea; Oxalis floribunda; Oxalis sp.

Papaveraceae: Argemone mexicana; Bocconia frutescens; Chelidonium majus; Chelidonium sp.; Dicentra sp.; Eschscholzia sp.; Fumaria officinalis; Papaver aculeatum; Papaver nudicaule; Papaver orientale; Papaver rhoeas; Papaver somniferum.

Passifloraceae: Passiflora caerulea; Passiflora edulis; Passiflora foetida; Passiflora mollissima; Passiflora sp.

Paulo wniaceae: Paulownia fortunei.

Pedaliaceae: Sesamum indicum.

Phyllanthaceae: Phyllanthus amarus; Phyllanthus sp.

Phytolaccaceae: Petiveria alliacea; Phytolacca americana; Phytolacca dioica; Phytolacca esculenta; Phytolacca icosandra.

Pinaceae: Pinus sylvestris; Tsuga canadensis.

Pittosporaceae: Pittosporum tobira.

Plantaginaceae: Angelonia sp.; Antirrhinum majus; Digitalis purpurea; Hippuris vulgaris; Linaria genistifolia; Mecardonia procumbens; Plantago asiatica; Plantago lanceolata; Plantago major; Plantago sp.; Veronica persica; Veronica sp.; Veronica teucrium.

Platanaceae: Platanus orientalis; Platanus sp.

Plumbaginaceae: Limoniastru guyonianum; Limonium sinuatum; Plumbago auriculata; Plumbago sp.

Poaceae: Aegilops sp.; Agropyron desertorum; Aira sp.; Avena fatua; Avena sativa; Avena sp.; Avena sterilis; Bambusa sp.; Bromus catharticus; Bromus sp.; Chondrosum barbatum; Cynodon dactylon; Dactyloctenium aegyptium; Digitaria argillacea; Digitaria ciliaris; Digitaria diversinervis; Digitaria sanguinalis; Eleusine coracana; Elymus hispidus; Elymus repens; Eragrostis sp.; Festuca arundinacea; Festuca sp.; Helictotrichon pratense; Hordeum sp.; Lolium multiflorum; Lolium sp.; Ophiuros exaltatus; Oryza glaberrima; Oryza sativa; Panicum miliaceum; Panicum sp.; Paspalum dilatatum; Pennisetum clandestinum; Pennisetum purpureum; Phleum pratense; Poa annua; Poa pratensis; Poa trivialis; Poaceae sp.; Rottboellia cochinchinensis; Saccharum officinarum; Setaria pumila; Setaria viridis; Sitanion hystrix; Sorghum bicolor; Sorghum halepense; Sorghum sp.; Stenotaphrum secundatum; Triticum sp.; Zea mays; Zeugites sp.

Polemoniaceae: Phlox Carolina; Phlox paniculata; Phlox sp.

Polygonaceae: Emex australis; Fallopia baldschuanica; Fallopia convolvulus; Persicaria hydropiper; Persicaria longiseta; Persicaria maculosa; Persicaria pensylvanica; Polygonum argyrocoleon; Polygonum aviculare; Rumex acetosa; Rumex acetosella; Rumex crispus; Rumex japonicus; Rumex obtusifolius; Rumex sp.

Pontederiaceae: Eichhornia crassipes.

Portulacaceae: Portulaca oleracea.

Primulaceae: Cyclamen graecum; Cyclamen hederifolium; Cyclamen persicum; Cyclamen sp.; Primula denticulata; Primula polyantha; Primula sp.; Primula veris.

Ranunculaceae: Adonis aestivalis; Anemone coronaria; Anemone hortensis; Aquilegia sp.; Clematis paniculata; Clematis sp.; Delphinium sp.; Helleborus sp.; Ranunculus asiaticus; Thalictrum fendleri.

Resedaceae: Reseda odorata.

Rhamnaceae: Frangula dodonei; Helinus integrifolius; Rhamnus alpina; Rhamnus imeretina; Ziziphus jujuba; Ziziphus spina-christi.

Rosaceae: Alchemilla vulgaris; Armeniaca mume; Cerasus lusitanica; Cerasus serrula; Cerasus vulgaris; Chaenomeles japonica; Chaenomeles sinensis; Cotoneaster horizontalis; Cotoneaster microphyllus; Cotoneaster tomentosa; Crataegus laevigata; Crataegus monogyna; Crataegus sanguinea; Cydonia oblonga; Eriobotrya japonica; Filipendula ulmaria; Fragaria moschata; Fragaria vesca; Fragaria virginiana; Fragaria x ananassa; Geum rivale; Malus domestica; Malus floribunda; Malus pumila; Malus sp.; Marcetella maderensis; Padus avium; Potentilla fragarioides; Potentilla fruticosa; Potentilla norvegica; Potentilla tanacetifolia; Prunus amygdalus; Prunus armeniaca; Prunus avium; Prunus cerasifera; Prunus cerasoides; Prunus cerasus; Prunus domestica; Prunus insititia; Prunus lusitanica; Prunus persica; Prunus salicina; Prunus serotina; Prunus sp.; Prunus spinosa; Pyracantha coccinea; Pyracantha koidzumii; Pyracantha sp.; Pyrus communis; Pyrus pyrifolia; Pyrus sp.; Rosa canina; Rosa cymosa; Rosa hybrida; Rosa multiflora; Rosa odorata; Rosa rugosa; Rosa sp.; Rosa x alba; Rosa x centifolia; Rosa x damascena; Rosa x rugosa; Rubus buergeri; Rubus chaerophyllus; Rubus chingii; Rubus fruticosus; Rubus idaeus; Rubus/loydianus; Rubus occidentalis; Rubus sp.; Rubus ulmifolius; Sorbus aucuparia; Sorbus sp.; Spiraea japonica.

Rubiaceae: Coffea arabica; Coffea abbayesii; Coffea affinis; Coffea alleizettii; Coffea ambanjensis; Coffea ambongenis; Coffea andrambovatensis; Coffea ankaranensis; Coffea anthonyi; Coffea arenesiana; Coffea augagneurii; Coffea bakossii; Coffea benghalensis; Coffea bertrandii; Coffea betamponensis; Coffea bissetiae; Coffea boinensis; Coffea boiviniana; Coffea bonnieri; Coffea brassii; Coffea brevipes; Coffea bridsoniae; Coffea buxifolia; Coffea canephora; Coffea carrissoi; Coffea charrieriana; Coffea cochinchinensis; Coffea commersoniana; Coffea congensis; Coffea costatifructa; Coffea coursiana; Coffea dactylifera; Coffea decaryana; Coffea dubardii; Coffea ebracteolata; Coffea eugenioides; Coffea fadenii; Coffea farafanganensis; Coffea floresiana; Coffea fotsoana; Coffea fragilis; Coffea fragrans; Coffea gallienii; Coffea grevei; Coffea heimii; Coffea homollei; Coffea horsfieldiana; Coffea humbertii; Coffea humblotiana; Coffea humilis; Coffea jumellei; Coffea kapakata; Coffea kianjavatensis; Coffea kihansiensis; Coffea kimbozensis; Coffea kivuensis; Coffea labatii; Coffea lancifolia; Coffea lebruniana; Coffea leonimontana; Coffea/eroyi; Coffea liaudii; Coffea liberica; Coffea ligustroides; Coffea littoralis; Coffea lulandoensis; Coffea mabesae; Coffea macrocarpa; Coffea madurensis; Coffea magnistipula; Coffea malabarica; Coffea mangoroensis; Coffea mannii; Coffea manombensis; Coffea mapiana; Coffea mauritiana; Coffea mayombensis; Coffea mcphersonii; Coffea melanocarpa; Coffea merguensis; Coffea millotii; Coffea minutiflora; Coffea mogenetii; Coffea mongensis; Coffea montekupensis; Coffea montis-sacri; Coffea moratii; Coffea mufindiensis; Coffea myrtifolia; Coffea namorokensis; Coffea neobridsoniae; Coffea neoleroyi; Coffea perrieri; Coffea pervilleana; Coffea pocsii; Coffea pseudozanguebariae; Coffea pterocarpan; Coffea racemose; Coffea rakotonasoloi; Coffea ratsimamangae; Coffea resinosa; Coffea rhamnifolia; Coffea richardii; Coffea sahafaryensis; Coffea sakarahae; Coffea salvatrix; Coffea sambavensis; Coffea sapinii; Coffea schliebenii; Coffea semsei; Coffea sessiliflora; Coffea stenophylla; Coffea tetragona; Coffea togoensis; Coffea toshii; Coffea travancorensis; Coffea tricalysioides; Coffea tsirananae; Coffea vatovavyensis; Coffea vavateninensis; Coffea vianneyi; Coffea vohemarensis; Coffea wightiana; Coffea zanguebariae; Galium aparine; Galium stellatum; Gardenia jasminoides; Gardenia sp.

Rutaceae: Choisya ternata; Citrus aurantiifolia; Citrus aurantium; Citrus Clementina; Citrus limon; Citrus maxima; Citrus medica; Citrus paradisi; Citrus reticulata; Citrus sinensis; Citrus sp.; Citrus trifoliata; Ruta graveolens; Zanthoxylum rhoifolium.

Salicaceae: Dovyalis caffra; Populus alba; Populus nigra; Populus sp.; Populus tremula; Populus x canadensis; Salix aegyptiaca; Salix alba; Salix babylonica; Salix caprea; Salix chaenomeloides; Salix dephnoides; Salix fragilis; Salix sp.; Salix viminalis.

Sapindaceae: Acer campestre; Acer negundo; Acer platanoides; Acer pseudoplatanus; Acer rubrum; Acer saccharum; Acer sp.; Aesculus glabra; Dodonaea viscosa; Koelreuteria paniculata; Litchi sinensis; Sapindus sp.

Saxifragaceae: Rodgersia podophylla.

Scrophulariaceae: Buddleja davidii; Buddleja madagascariensis; Diascia sp.; Myoporum sp.; Nemesia sp.; Verbascum blattaria.

Simaroubaceae: Ailanthus altissima.

Solanaceae: Acnistus arborescens; Brugmansia arborea; Brugmansia suaveolens; Brugmansia x Candida; Calibrachoa sp.; Capsicum annuum; Capsicum sp.; Cestrum cyaneum; Cestrum elegans; Cestrum strigillatum; Cyphomandra sp.; Datura metel; Datura sp.; Datura stramonium; Lycium chinense; Nicandra physalodes; Nicotiana glauca; Nicotiana sp.; Nicotiana tabacum; Petunia sp.; Petunia x hybrid; Physalis acutifolia; Physalis alkekengi; Physalis angulata; Physalis lagascae; Physalis peruviana; Salpichroa origanifolia; Solanum aethiopicum; Solanum americanum; Solanum capsicoides; Solanum carolinense; Solanum delagoense; Solanum elaeagnifolium; Solanum grandiflorum; Solanum laciniatum; Solanum lycopersicum; Solanum macrocarpon; Solanum mammosum; Solanum melongena; Solanum muricatum; Solanum nigrum; Solanum panduraeforme; Solanum quitoense; Solanum sp.; Solanum tuberosum; Withania somnifera.

Strelitziaceae: Strelitzia reginae.

Theaceae: Camellia japonica; Camellia sinensis; Camellia sp.

Thymelaeaceae: Dais cotinifolia.

Tropaeolaceae: Tropaeolum majus; Tropaeolum sp.

Ulmaceae: Ulmus americana; Ulmus glabra; Ulmus laevis; Ulmus pumila; Ulmus rubra; Ulmus sp.

Urticaceae: Boehmeria nivea; Laportea aestuans; Parietaria judaica; Parietaria officinalis; Pipturus albidus; Urtica dioica; Urtica sp.; Urtica urens.

Verbenaceae: Aloysia citriodora; Duranta erecta; Glandularia phlogiflora; Lantana camara; Lippia alba; Verbena bracteata; Verbena brasiliensis; Verbena hybrida; Verbena officinalis; Verbena sp.

Violaceae: Viola odorata; Viola sp.; Viola tricolor; Viola x wittrockiana.

Vitaceae: Ampelopsis sp.; Parthenocissus quinquefolia; Parthenocissus tricuspidata; Vitis sp.; Vitis vinifera.

Xanthorrhoeaceae: Hemerocallis fulva; Hemerocallis minor.

Zingiberaceae: Curcuma longa; Zingibermioga.

Zygophyllaceae: Tribulus terrestris.

In particular embodiments, the LC is from a plant in the plant family Cannabaceae, Rubiaceae, and/or Theaceae.

In particular embodiments, the LC is from a plant in the Cannabaceae family, for example, from a cannabis plant including Cannabis sativa, Cannabis indica, and/or Cannabis ruderalis.

There are hundreds of strains of Cannabis sativa including: Amnesia Haze which is a sativa strain that features up to 25 percent THC with long-lasting body-relaxing and clear-headed effects that are beneficial for dealing with mood problems and tension; Sour Diesel offers 19 percent THC and provides users with an invigorating, yet dreamy cerebral effect; Strawberry Cough has a pungent strawberry scent and tendency to make the consumer cough and contains 24 to 26 percent THC; Jack Herer features between 17 and 18 percent THC and causes users to feel an overwhelming feeling of bliss, giving them a clear head that is ready to tackle a creative project; Tangie or Tangerine Dream is 17 percent THC and provides users with a perfect mix of euphoria and relaxation; Super Lemon Haze is an energizing sativa strain that is 18 percent THC which gives users a jolt of energy after use; and Maui Wowie features sweet pineapple flavors and 18 percent THC giving users a motivated, creative feeling.

In particular embodiments, the LC is from a hops plant including a Humulus plant including Humulus lupulus, Humulus japonicus, Humulus americanus, Humulus cordifolius, Humulus neomexicanus, Humulus pubescens, and/or Humulus scandens.

There are several varieties of hops around the world. American varieties include: Ahtanum brand YCR 1 cv is an aroma-type cultivar used for its moderate bittering (Shop, Brooklyn Brew. “Hop Profile: Ahtanum”. Brooklyn Brew Shop); Amarillo brand VGXPO1 cv is a mid-range alpha acid variety with a unique and distinct aroma discovered growing “wild” (spontaneously) in a hop yard and developed by Virgil Gamache Farms in the late 20th century; Apollo is characterized by its exceptionally high percentage of alpha acids (15.0-19.0%), excellent storage stability of alpha acids, low CoHumulone value for an alpha variety (24-28%), and resistance to hop powdery mildew strains found in Washington; Azacca has predominately citrus and tropical fruit notes, including mango, papaya, orange, grapefruit, lemon, piney, spicy, pineapple, grassy, tropical fruit, citrus aromas; Calypso is a dual-purpose high alpha hop with aromas of pear, apple and earthy tea; Cascade is an aroma hop developed by USDA-ARS's breeding program in 1956 from Fuggle and Serebrianker; Centennial is an aroma-type variety bred in 1974 with a genetic composition of ¾ Brewers Gold, 3/32 Fuggle, ⅙ Golding, 1/32 Bavarian and 1/16 Unknown; Chinook is a green bine cultivar (W-421-38) from Washington State and Idaho that is a cross between a Petham Golding and a USDA-selected male (63012M); Citra brand HBC 394 cv is a registered trademark used with HBC 394 cv special aroma hop variety developed by the Hop Breeding Company (Bernstein, Josh M. (Jun. 17, 2010). “New Hops Breeds”. Imbibe Magazine.); Cluster originated from mass selection of the Cluster hop, which is an old American cultivar; Columbus is a high yielding, high alpha acid American bittering hop known by the trade name Tomahawk; Comet is a rare US grown hop, originally bred for its bittering characteristics; Crystal is an American triploid variety developed in 1993 from Hallertau, Cascade, Brewer's Gold and Early Green; El Dorado is a high alpha hop developed by CLS Farms; Ekuanot is a high oil content hop known for strong aroma including fruitiness, eucalyptus, clove, and tobacco formerly known as Equinox (“Ekuanot®”. BSG CraftBrewing); Eroica has a pale green bine was bred from Brewers Gold with an open pollination; Galena is a cultivar developed in 1968 from Brewer's Gold by open pollination in Idaho; Glacier is a low-cohumulone American Fuggle descendant; Greensburg is grown in southern Idaho; Horizon is a high alpha cross made in Oregon in 1970; Liberty is a cross between Hallertauer Mittlefrüh (USDA 21397) and downy mildew resistant male (USDA 64035M); Mount Hood is a variety developed from Hallertau used in styles that require only a subtle hop aroma (German/American lagers); Mount Rainier has a complex parentage, including Hallertau, Galena, Fuggle and other hops, and exhibits some noble hop characteristics, but is higher in alpha acid; Mosaic® (Hop Breeding Company, Yakima, WA) Brand HBC 369 cv is a daughter of the YCR 14 cv hop variety and a Nugget derived male; Newport is a high-alpha bittering hop; Nugget has a floral, resiny aroma and flavor; Palisade brand YCR 4 cv hop variety is an aroma hop that is known for its amazing yield and unique aroma; San Juan Ruby Red was discovered growing wild in the San Juan Mountains of Colorado; Santiam is a floral aroma hop with mid-range alpha acid with a pedigree including Tettnang (mother), Hallertau Mittelfrüh (grandmother) and Cascade (great grandmother); Satus is a bittering-type cultivar produced by Yakima Chief Ranches in Washington State (“Satus Hops|Where legends are made”. Beerlegends.com); Simcoe brand YCR 14 cv is an aroma variety known for its versatility and unique characteristics with several different aromas including passion fruit, pine, earthy, and citrus; Sonnet Golding is a low alpha American-grown Golding variety; Sterling is a floral hop which is a cross between Saaz and Mount Hood in character but easier to grow; Summit is useful for barleywines, stouts and IPAs with alpha acids of 17-19% and beta acids of 4.0-6.0%; Tomahawk is the trade name for Columbus; Ultra is a triploid aroma-type cultivar that is a cross between the colchicine-induced tetraploid Hallertau mf (USDA 21397) and the diploid Saazer-derived male genotype (USDA 21237m); Vanguard is an aroma cross developed from Hallertau in 1982; Warrior brand YCR 5 cv hop variety is an alpha hop used for its mild, clean bittering qualities; Willamette is an American development in 1976 of the English Fuggle (“KSWbeer.com: Ahtanum Hops”. www.kswbeer.com); Zeus is an aromatic high-alpha hop; and Zythos is a blend of Simcoe, Citra, Palisade and Amarillo, having distinct tropical (pineapple) and citrus tones, with slight pine characteristics.

British hop varieties include: Admiral is a bittering hop used in some English ales; Boadicea is a spicy, light, floral hop suitable for finishing and dry hopping; Bramling Cross is a cross between the Bramling goldings variety and a wild Canadian Manitoban hop; Brewer's Gold is a bittering hop with a resiny, spicy aroma/flavor with hints of black currant; Bullion is a bittering hop with a resiny/earthy aroma/flavor; Challenger is used in many traditional English Bitters with pleasant and complex marmalade/toffee/citrus flavour, which enhances strong ales; First Gold is the first English commercial grown dwarf hop, released in 1995 and bred by Wye College in Kent; Fuggle is a classic English aroma hop which is known as Styrian Goldings in Europe and is a parent of many New World hops such as Cascade, Centennial and Willamette; Goldings are a group of related clones that harvest at different times such as Cobbs, Amos' Early Bird, Eastwell, Mathon, East Kent and historical clones such as Bramling, Canterbury, Rodmersham and Petham (“Goldings” British Hop Association, www.britishhops.org.uk/varieties/goldings); Herald is an English aroma and bittering hop; Northdown is a dual purpose hop in England developed in the 1970s; Northern Brewer is a cross between a Canterbury Golding female plant and the male plant OB21 (“Northern Brewer Hops”. www.brew-dudes.com; and “YC Hop Varieties”. www.yakimachief.com); Phoenix is a dual-purpose English hop with a mild aroma and slightly spicy flavor (“Phoenix Hops”. www.brew-dudes.com/phoenix-hops/179); Pilgrim was bred at the Horticulture Research International in Wye, England. Released 2000. Dual purpose with lemon tones; Pilot was previously known as S24; Pioneer is an English hop with distinct bitter lemon flavor; Progress is an English hop developed in the 1960s as a replacement for Fuggle; Target is an English mid-to-high alpha hop bred from Kent Goldings; and Whitbread Golding Variety (WGV) was bred in 1911 from open pollination of Bates Brewer.

German hop varieties include: Hallertauer Magnum is a high alpha acid type cultivar bred from the American variety Galena with a German male hop (75/5/3); Hallertauer Merkur was bred from a cross between Hallertaur Magnum and variety 81/8/13 (“Merkur Hops|Where legends are made”. Beerlegends.com); Hallertauer Taurus is a high to super-high alpha bittering hop (“Hallertauer Taurus” Hopsteiner. web.archive.org/web/20030514092954/www.hopsteiner.com/pdf/germany/HallertauerTaurus.PDF); Hallertauer Tradition was bred in 1991 for resistance to disease; Herkules is a cross between Hallertauer Taurus and a powdery mildew resistant Hull male breeding line; Huell Melon is an aroma hop variety featuring fruity honeydew melon and apricot aroma (“Annual Report 2013—Special Crop: Hops”. Bavarian State Research Center for Agriculture (LfL). March 2014. p. 45.; and Lutz, Anton; Kammhuber, Klaus; Seigner, Elisabeth (2012). “New Trend in Hop Breeding at the Hop Research Center Huell”. BrewingScience. 65 (¾): 24-32); Mandarina Bavaria is an aroma hop variety featuring fruity mandarin orange and citrus aroma (“Annual Report 2013—Special Crop: Hops” Bavarian State Research Center for Agriculture (LfL). March 2014. p. 45.; and Lutz, Anton; Kammhuber, Klaus; Seigner, Elisabeth (2012). “New Trend in Hop Breeding at the Hop Research Center Huell”. BrewingScience. 65 (¾): 24-32); Opal has a sweet spice, slightly peppery aroma and flavor combined with a light, clean citrus fruit aroma (“Opal”. Hopslist); Perle is a dual-purpose hop with spicy and slightly floral/fruity; Polaris is an aroma hop variety with high bittering potential and fruity/spicy, fresh aroma reminiscent of mint and menthol (“Annual Report 2012—Special Crop: Hops”. Bavarian State Research Center for Agriculture (LfL). March 2013; “Annual Report 2013—Special Crop: Hops”. Bavarian State Research Center for Agriculture (LfL). March 2014. p. 45; and Lutz, Anton; Kammhuber, Klaus; Seigner, Elisabeth (2012). “New Trend in Hop Breeding at the Hop Research Center Huell”. BrewingScience. 65 (¾): 24-32); Saphir shares many characteristics with the Hallertauer Mittelfrüh; Smaragd is a mid-alpha hop with fruity, floral characteristic; and Spalter Select is a disease-resistant Hallertauer and Spalt pale lager variety developed in the early 1990s.

Other European hop varieties include: Aalst is a Belgian variety (Lacambre, G. Traité de la Fabrication des Bières et de la Distillation des Grains, etc. Vol. 1. 1851); Aramis is a French hop used for bittering and aroma (“Alsace Aramis Hop—Aramis Hops for sale—Comptoir Agricole”); Bor is a hybrid of Saaz and Northern Brewer (“Bor”. Hopsteiner. web.archive.org/web/20040706065911/www.hopsteiner.com/pdf/europe/Bor.pdf); Coigneau is a Belgian hop with low bitterness; lunga is a variety from IUNG Pulawy (“Global Hops—Hops Varieties”); Lublin is a Polish grown Saaz, used in Polish lagers; Marynka is a bittering hop with an earthy, licorice quality; Poperinge is a Belgian variety mentioned for its use in Lambic beer in the early 19th century (Lacambre, G. Traité de la Fabrication des Bières et de la Distillation des Grains, etc. Vol. 1. 1851); Premiant is a high-alpha Saaz variant. Gives similar spicy aroma qualities (“Premiant”. Hopsteiner. web.archive.org/web/20050223031436/www.hopsteiner.com/pdf/europe/Premiant.pdf); Sladek is a hybrid of Northern Brewer, Osvald's close No. 126 and Czech male components. Similar to Czech Saaz (“Sladek”. Hopsteiner. web.archive.org/web/20050223035515/www.hopsteiner.com/pdf/europe/Sladek.pdf); Strisselspalt is a French aroma hop from Alsace, used mostly in pale lagers; Styrian Atlas is a diploid hybrid between Brewers Gold and 3/3 Slovenian wild hop; Styrian Aurora is a diploid hybrid between Northern Brewer and a TG seedling of unknown origin (Taish, Bostjan. “Aurora Data Sheet”); Styrian Bobek is a delicate, spicy aroma hop (Taish, Bostjan. “Bobek Data Sheet”); Styrian Celeia gives a pleasant hoppy aroma similar to other “Super Styrian” varieties (Taish, Bostjan. “Celeia Data Sheet”); Styrian Golding is a Slovenian variant used in English ales and Belgian strong ales; Sybilla is a variety from IUNG Pulawy which is registered as a bittering hop, but has aroma characteristics (“Global Hops—Hops Varieties”); Tardif de Bourgogne is a French hop, used as an aromatic in continental lagers; and Tomyski is a Polish variety used as an aroma hop.

Noble hop varieties include: Hallertau or Hallertauer Mittelfrüh was the original German lager hop; Hersbrucker used in German pale lagers; Saaz used to flavor pale Czech lagers; Tettnanger used in European pale lagers and wheat beers; and Spalt which is has a woody taste.

Australian hop varieties include: Ella is a high alpha variety formerly known as Stella with breeding code 01-220-060 (“Ella”. Hop Products Australia); Enigma is a high alpha variety typically 16-19% which is decedent from the Swiss Tettnang hop (“Hop Products Australia Enigma™); Feux-Coeur Francais has its genetic roots in the Burgundy region of France; Galaxy is a high alpha dual purpose triploid cultivar with a marked and unique hop aroma, described as a combination of citrus and passionfruit bred by crossing a female tetraploid (J78) with a male derived from Perle (“Hop Products Australia>Products>Australian Varieties>Galaxy”. Hops.com.au); Pride of Ringwood is a hops used by Australian brewer Carlton and United Breweries to bitter all of its beers; Summer is a low alpha Australian aroma variety with breeding code 97-235-026 (“Hop Products Australia>Products>Australian Varieties>Summer”. Hops.com.au); Super Pride is a high alpha version of Pride of Ringwood, bred by crossing a female tetraploid Pride of Ringwood with a male YK¬81¬18 (“Hop Products Australia>Products>Australian Varieties>Super Pride”. Hops.com.au); Topaz is a high alpha Australian variety bred by crossing a female Tetraploid J78 with a male 29/70/54; and Vic Secret is a hop with breeding code 00-207-013.

New Zealand hop varieties include: Green Bullet is a triploid Alpha Variety bred by open cross-pollination of the New Zealand “Smoothcone” variety (“New Zealand Hops Limited—Supplier of hops, commercial brewing supplies, home brewing supplies, spray free and organic Bio-Gro hops”. Nzhops.co.nz); Hallertau Aroma/Wakatu was released commercially from Hort's Riwaka Research Centre in 1988 (“New Zealand Hops Limited—Supplier of hops, commercial brewing supplies, home brewing supplies, spray free and organic Bio-Gro hops”. Nzhops.co.nz); Kohatu is a mid-alpha variety with aromas of pine needles and tropical fruit; Motueka was bred by crossing a New Zealand breeding selection (⅔) with Saazer parentage (⅓) (“New Zealand Hops Limited—Supplier of hops, commercial brewing supplies, home brewing supplies, spray free and organic Bio-Gro hops”. Nzhops.co.nz); Nectaron is a triploid aroma type developed in collaboration with New Zealand's Plant & Food Research (“NZ Hops reunites with Plant & Food for hop research”. Brews News. 2020-10-23; and “NZ hops capturing world attention”. NZ Herald.); Nelson Sauvin is a variety developed in Nelson, New Zealand in 1987 and commercially available since 2000 (Beatson/Ansell/Graham: “Breeding, development, and characteristics of the hop (Humulus lupulus) cultivar ‘Nelson Sauvin’, New Zealand Journal of Crop and Horticultural Science, 2003, Vol. 31: 303-309); New Zealand Cascade was bred from crossing an English Fuggle with a male selection believed to have been a crossing of Fuggle with the Russian variety Serebrianka; Pacifica was previously known as the Pacific Hallertau; Pacific Gem is a triploid Alpha type bred from the New Zealand variety “Smoothcone” crossed with Californian Late Cluster x Fuggle (“New Zealand Hops Limited—Supplier of hops, commercial brewing supplies, home brewing supplies, spray free and organic Bio-Gro hops”. Nzhops.co.nz); Pacific Jade is a high alpha bittering hop from New Zealand with a soft bitterness; Rakau is a dual purpose variety typically with alpha acid above 10% and cohumulone less than 25% of alpha acids; Riwaka is a triploid aroma type bred by crossing “Old Line” Saazer with specially developed New Zealand breeding selections; Southern Cross is a mellow bittering hop with high alpha of 11.0-14.0%; Sticklebract is a triploid variety with aroma of pine and citrus; Super Alpha is a triploid variety bred from the New Zealand Smoothcone variety cross open pollinated at the New Zealand Horticultural Research Centre (now known as HortResearch) and released in 1976 (New Zealand Hops Limited—Supplier of hops, commercial brewing supplies, home brewing supplies, spray free and organic Bio-Gro hops”. Nzhops.co.nz); Wai-iti is a low-alpha variety with a strong citrus character of mandarin, lemon and lime zest; and Waimea is a granddaughter of Pacific Jade and commercialised on dual purpose capability.

A Japanese hop includes Sorachi Ace which is a dual-purpose hop with a lemon, bubblegum, and dill pickle flavor.

An ornamental hop includes Golden hops which is a pale, ornamental variety, Humulus lupulus ‘Aurea’, and is cultivated for garden use.

In particular embodiments, the LC is from the Rubiaceae family and is a Coffea plant including Coffea abbayesii, Coffea affinis, Coffea alleizettii, Coffea ambanjensis, Coffea ambongenis, Coffea andrambovatensis, Coffea ankaranensis, Coffea anthonyi, Coffea arabica, Coffea arenesiana, Coffea augagneurii, Coffea bakossii, Coffea benghalensis, Coffea bertrandii, Coffea betamponensis, Coffea bissetiae, Coffea boinensis, Coffea boiviniana, Coffea bonnieri, Coffea brassii, Coffea brevipes, Coffea bridsoniae, Coffea buxifolia, Coffea canephora, Coffea carrissoi, Coffea charrieriana, Coffea cochinchinensis, Coffea commersoniana, Coffea congensis, Coffea costatifructa, Coffea coursiana, Coffea dactylifera, Coffea decaryana, Coffea dubardii, Coffea ebracteolata, Coffea eugenioides, Coffea fadenii, Coffea farafanganensis, Coffea floresiana, Coffea fotsoana, Coffea fragilis, Coffea fragrans, Coffea gallienii, Coffea grevei, Coffea heimii, Coffea homollei, Coffea horsfieldiana, Coffea humbertii, Coffea humblotiana, Coffea humilis, Coffea jumellei, Coffea kapakata, Coffea kianjavatensis, Coffea kihansiensis, Coffea kimbozensis, Coffea kivuensis, Coffea labatii, Coffea lancifolia, Coffea lebruniana, Coffea leonimontana, Coffea leroyi, Coffea liaudii, Coffea liberica, Coffea ligustroides, Coffea littoralis, Coffea lulandoensis, Coffea mabesae, Coffea macrocarpa, Coffea madurensis, Coffea magnistipula, Coffea malabarica, Coffea mangoroensis, Coffea mannii, Coffea manombensis, Coffea mapiana, Coffea mauritiana, Coffea mayombensis, Coffea mcphersonii, Coffea melanocarpa, Coffea merguensis, Coffea millotii, Coffea minutiflora, Coffea mogenetii, Coffea mongensis, Coffea montekupensis, Coffea montis-sacri, Coffea moratii, Coffea mufindiensis, Coffea myrtifolia, Coffea namorokensis, Coffea neobridsoniae, Coffea neoleroyi, Coffea perrieri, Coffea pervilleana, Coffea pocsii, Coffea pseudozanguebariae, Coffea pterocarpan, Coffea racemose, Coffea rakotonasoloi, Coffea ratsimamangae, Coffea resinosa, Coffea rhamnifolia, Coffea richardii, Coffea sahafaryensis, Coffea sakarahae, Coffea salvatrix, Coffea sambavensis, Coffea sapinii, Coffea schliebenii, Coffea semsei, Coffea sessiliflora, Coffea stenophylla, Coffea tetragona, Coffea togoensis, Coffea toshii, Coffea travancorensis, Coffea tricalysioides, Coffea tsirananae, Coffea vatovavyensis, Coffea vavateninensis, Coffea vianneyi, Coffea vohemarensis, Coffea wightiana, and/or Coffea zanguebariae.

In particular embodiments, an LC is from the Theaceae family and is a tea plant including Camellia sinensis. The variants of Camellia sinensis include Camellia sinensis var. sinensis, Camellia sinensis var. assamica, Camellia sinensis var. pubilimba, or Camellia sinensis var. dehungensis. In particular embodiments, tea varieties include black tea, green tea, white tea, oolong tea, pu-erh tea, and purple tea which are all made from the Camellia sinensis tea plant. Camellia sinensis var. sinensis grows primarily in China and other East Asian countries and has a milder, mellower character, and Camellia sinensis var. assamica grows primarily in India and is generally heartier and more robust. Other varieties include Camellia sinensis var. pubilimba and Camellia sinensis. var. dehungensis (in T, Bartholomew B. “18. Theaceae”. Flora of China. 12).

As indicated, particles, aqueous dispersions, and compositions described here include an LC. In particular embodiments, an LC is a first LC that is chosen for an intended physiological effect (e.g., the first LC is an active compound or an active ingredient). In particular embodiments, a first LC includes a cannabinoid; α-acids (e.g., humulone, cohumulone, adhumulone, posthumulone, prehumulone, adprehumulone) β-acids (e.g. lupulone, colupuloe, adlupulone, prelupulone, postlupulone), or flavonoids (e.g. xanthohumol, or xanthan humulone) from a hops plant; tryptophan alkaloids or diterpenes from a coffee plant; resveratrol, or proanthocyanidins from a tea plant; or coenzyme Q10 (ubiquinone or ubiquinol), plastoquinone, plastoquinonol, or other fat-soluble electron transport chain components found in the mitochondria of plants (and all respiring eukaryotic cells).

In particular embodiments, an LC can come from multiple plant types. For example, resveratrol can be found in tea but can also be found in peanuts, grapes, wine, and soy.

As used herein, the term “cannabinoid” is generally understood to include any chemical compound that acts upon a cannabinoid receptor. For instance, cannabinoids may include endocannabinoids (i.e., produced naturally by humans and animals), phytocannabinoids (i.e., found in cannabis and some other plants), and artificial cannabinoids (i.e., manufactured and not naturally occuring).

Examples of cannabinoids include cannabidiol (CBD), cannabigerolic acid (CBGA), cannabigerol (CBG), cannabigerol monomethylether (CBGM), cannabigerovarin (CBGV), cannabichromene (CBC), cannabichromevarin (CBCV), cannabidiol monomethylether (CBDM), cannabidiol-C4 (CBD-C4), cannabidivarin (CBDV), cannabidiorcol (CBD-C1), Δ-9-tetrahydrocannabinol (A9-THC), Δ-9-tetrahydrocannabinolic acid A (THCA-A), Δ-9-tetrahydrocannabionolic acid B (THCA-B), Δ-9-tetrahydrocannabinolic acid-C4 (THCA-C4), Δ-9-tetrahydrocannabinol-C4, Δ-9-tetrahydrocannabivarin (THCV), Δ-9-tetrahydrocannabiorcol (THC-C1), Δ-7-cis-iso tetrahydrocannabivarin, Δ-8-tetrahydrocannabinol (Dd-THC), cannabicyclol (CBL), cannabicyclovarin (CBLV), cannabielsoin (CBE), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 10-ethoxy-9hydroxy-Δ-6a-tetrahydrocannabinol, 8,9-dihydroxy-Δ-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-Δ-6a-tetrahydrocannabionol (OTHC), Δ-9-cis-tetrahydrocannabinol (cis-THC), 3,4,5,6-tetrahydro-7-hydroxy-α-α-2-trimethyl-9-n-propyl-2, 6-methano-2H-1-benzoxocin-5-methanol (OH-iso-HHCV), cannabiripsol (CBR), trihydroxy-Δ-9-tetrahydrocannabinol (triOH-THC), cannabinol propyl variant (CBNV), cannabidiolic acid (CBDA), cannabitriol (CBO), and tetrahydrocannabivarinic acid (THCVA) and derivatives thereof. Further examples of cannabinoids are discussed in PCT Patent Application Pub. No. WO2017/190249 and U.S. Patent Application Pub. No. US2014/0271940.

The term “derivative” in chemistry refers to a compound that is obtained from a similar compound or a precursor compound by a chemical reaction.

Examples of cannabinoids that can be synthetically produced include: naphthoylindoles, naphthylmethylindoles, naphthoylpyrroles, naphthylmethylindenes, phenylacetylindoles, cyclohexylphenols, tetramethylcyclopropylindoles, adamantoylindoles, indazole carboxamides, and quinolinyl esters. Derivatives of natural cannabinoids can include metabolites of cannabinoids which are disclosed in WO 2015/198078. For example, the metabolite of CBD includes 7-OH-CBD and the metabolite of CBDV includes 7-OH-CBDV. In particular embodiments, examples of cannabinoids include 3-carbamoyl-2-pyridone, and its derivatives and/or analogs disclosed in US 2008/0103139; pyrimidine derivatives and/or analogs disclosed in US 2006/0293354; carenadiol and its derivatives and/or analogs thereof disclosed in U.S. Pat. No. 4,758,597; cannabinoid carboxylic acids and their derivatives and/or analogs disclosed in WO 2013/045115; pyrido[3,2-E][1,2,4]triazolo[4,3-C]pyrimidine and its derivatives and/or analogs disclosed in WO 2008/118414; tetrahydro-pyrazolo[3,4-C] pyridine and its derivatives and/or analogs disclosed in WO 2007/112399; bicyclo[3.1.1]heptan-2-one cannabinoid and its derivatives and/or analogs disclosed in WO 2006/043260; resorcinol and its derivatives and/or analogs disclosed in WO 2005/0123051; dexanabinol compounds and their derivatives and/or analogs disclosed in WO 2004/050011; cannabimimetic lipid amide compounds and their derivatives and/or analogs disclosed in WO 2000/032200; nabilone and its derivatives and/or analogs disclosed in US 2010/0168066; 2-oxoquinolone compounds and their derivatives and/or analogs disclosed in US 2003/0191069; and 3,4-diaryl-4,5-dihydro-(h)-pyrazole-1-carboxamide and its derivatives and/or analogs disclosed in US 2011/0137040.

In particular embodiments, 3-carbamoyl-2-pyridone and its derivatives and/or analogs include methyl 3-methyl-2-{[2-oxo-1-(2-oxo-ethyl)-1,2,5,6,7,8,9,10-octahydro-cycloocta[b]pyridine-3-carbonyl]-amino}-butyrate; dimethyl 2-[(1-cyclohexylmethyl-5,6-dimethyl-2-oxo-1,2-dihydropyridine-3-carbonyl)-amino]-succinate; and methyl 2-{[1-(3-methoxycarbonyamino-propyl)-2-oxo-1,2,5,6,7,8,9,10-octahydro-cycloocta[b]pyridine-3-carbonyl]-amino}-2-methyl-propionate.

In particular embodiments, pyrimidine derivatives and/or analogs include a compound having Formula (I) (2-((2,4-dichlorophenyl)amino)-N-((tetrahydro-2H-pyran-4-yl)methyl)-4-(trifluoromethyl)pyrimidine-5-carboxamide),

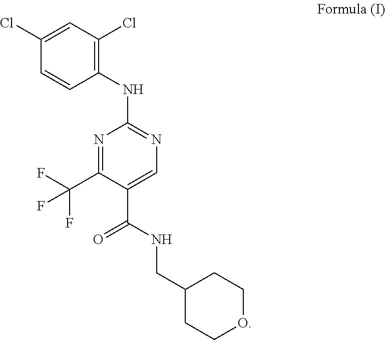 [SMILES: O=C(NCC1CCOCC1)c1cnc(Nc2ccc(Cl)cc2Cl)nc1C(F)(F)F]

Other pyrimidine derivatives and/or analogs include 2-(3-Chlorophenylamino)-4-trifluoromethylpyrimidine-5-carboxylic acid cyclohexylmethyl-amide; 2-Phenylamino-4-trifluoromethylpyrimidine-5-carboxylic acid cyclohexylmethyl-amide; 1-[2-(2,3-Dichlorophenylamino)-4-trifluoromethylpyrimidin-5-yl]-1-morphol-in-4-yl-methanone; 1-[2-(2,4-Dichlorophenylamino)-4-trifluoromethylpyrimidin-5-yl]-1-morphol-in-4-yl-methanone; and 2-(3-Chlorophenylamino)-4-trifluoromethylpyrimidin-5-carboxylic acid cyclopentylamide.

In particular embodiments, carenadiol and its derivatives and/or analogs include compounds having Formula II),

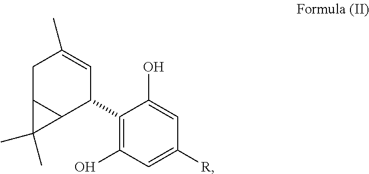

wherein R is a lower alkyl having 1 to 9 carbon atoms including isomeric forms such as i-butyl, n-butyl, and t-butyl. In particular embodiments, R is C₅H₁₁ or 1,1-dimethylheptyl.

In particular embodiments, cannabinoid carboxylic acids and their derivatives and/or analogs include compounds having Formula (III), (IV), (V), or (VI),

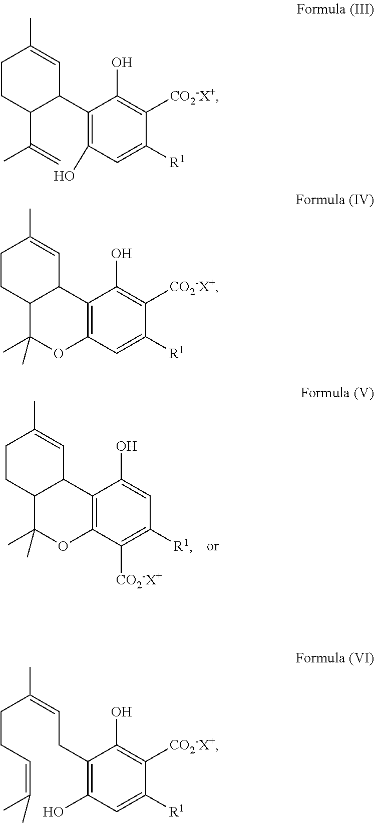

wherein:
R¹ is a straight-chain, branched or cyclic hydrocarbon residue with one C atom to 12 C atoms; and
X⁺ is NH₄⁺, mono-, di- or trivalent metal ions; or primary, secondary, tertiary or quaternary organic ammonium ions with up to 48 C atoms, which may bear still further functional groups.

Examples of multivalent ammonium ions include N,N-dicyclo-hexylamine-H+ and N,N-dicyclohexyl-N-ethylamine-H⁺. X⁺ can also be the hydrogen cation of a physiologically active substance with at least one basic nitrogen atom, such as for example morphine, methadone (or an enantiomer thereof) or hydromorphone.

In particular embodiments, pyrido[3,2-E][1,2,4]triazolo[4,3-C]pyrimidine and its derivatives and/or analogs include 5-tert-butyl-8-(2-chlorophenyl)-9-(4-chlorophenyl)pyrido[3,2-e][1,2,4]triazolo[4,3-c]pyrimidin-3(2H)-one; 8-(4-bromo-2-chlorophenyl)-5-tert-butyl-9-(4-chlorophenyl)pyrido[3,2-e][1,2,4]triazolo[4,3-c]pyrimidin-3(2H)-one; 5-tert-butyl-9-(4-chlorophenyl)-8-(2-methylphenyl)pyrido[3,2-e][1,2,4]triazolo[4,3-c]pyrimidin-3(2H)-one; 9-(4-bromophenyl)-5-tert-butyl-8-(2-chlorophenyl)pyrido[3,2-e][1,2,4]triazolo[4,3-c]pyrimidin-3(2H)-one; and 5-tert-butyl-8-(2-chlorophenyl)-9-(4-chlorophenyl)pyrido[3,2-e][1,2,4]triazolo[4,3-c]pyrimidine.

In particular embodiments, tetrahydro-pyrazolo[3,4-C] pyridine and its analogs and/or derivatives include compounds having Formula (VII), (VIII), (IX), (X), or (XI),

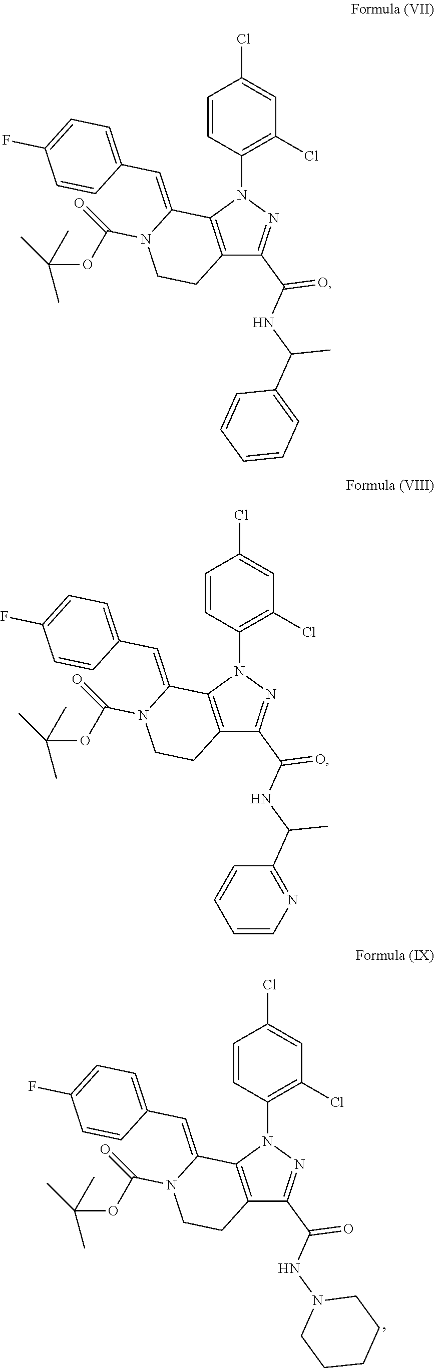

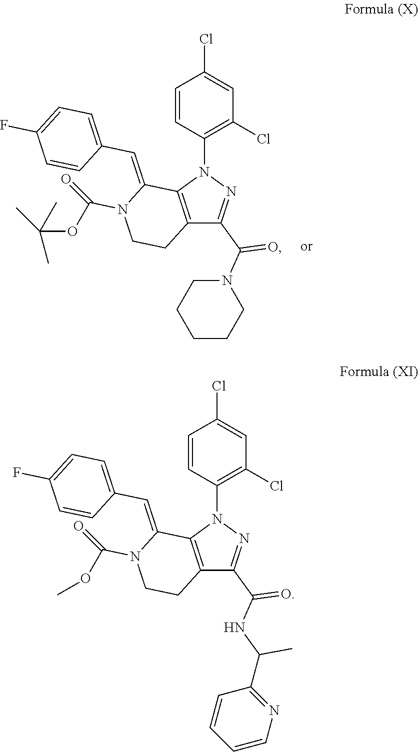 [SMILES: CC(C)(C)OC(=O)N1CCc2c(C(=O)N3CCCCC3)nn(-c3ccc(Cl)cc3Cl)c2/C1=C/c1ccc(F)cc1 ; COC(=O)N1CCc2c(C(=O)NC(C)c3ccccn3)nn(-c3ccc(Cl)cc3Cl)c2/C1=C/c1ccc(F)cc1]

In particular embodiments, bicyclo[3.1.1]heptan-2-one cannabinoids and their derivatives and/or analogs include compounds having Formula (XII),

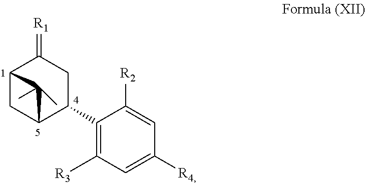

having a specific stereochemistry wherein C-4 is S, the protons at C-1 and C-5 are cis in relation to one another and the protons at C-4 and C-5 are trans; and wherein:
R₁ is (a) O or S; (b) C(R′)₂ wherein R′ at each occurrence is independently selected from the group consisting of hydrogen, cyano, —OR″, —N(R″)₂, a saturated or unsaturated, linear or branched C₁-C₆ alkyl, C₁-C₆ alkyl-OR″ or C₁-C₆alkyl-N(R″)₂ wherein at each occurrence R″ is independently selected from the group consisting of hydrogen, C(O)R′″, C(O)N(R′″)₂, C(S)R′″, saturated or unsaturated, linear or branched C₁-C₆ alkyl, C₁-C₆ alkyl-OR′″, and C₁-C₆ alkyl-N(R′″)₂, wherein at each occurrence R′″ is independently selected from the group consisting of hydrogen or saturated or unsaturated, linear, branched or cyclic C₁-C₁₂alkyl; or (c) NR″ or N—OR″ wherein R″ is as previously defined;
R₂ and R₃ are each independently (a) —R″, —OR″, —N(R″)₂, —SR″, —S(O)(O)NR″, wherein at each occurrence R″ is as previously defined; (b) —S(O)R^{b}, —S(O)(O)R^{b} wherein R^{b} is selected from the group consisting of hydrogen, saturated or unsaturated, linear or branched C₁-C₆alkyl, C₁-C₆alkyl-OR″, and C₁-C₆alkyl-N(R″)₂, wherein R″ is as previously defined; or (c) —OC(O)OH, —OS(O)(O)OR^{e}, —OP(O)(OR^{e})₂, —OR^{d} or —OC(O)—R^{d} chain terminated by —C(O)OH, —S(O)(O)OR^{e}, or —P(O)(OR^{e})₂, wherein R^{d} is a saturated or unsaturated, linear or branched C₁-C₆ alkyl and Re is at each occurrence selected from the group consisting of hydrogen and R^{d} as previously defined; and
R₄ is (a) R wherein R is selected from the group consisting of hydrogen, halogen, OR′″, OC(O)R′″, C(O)OR′″, C(O)R′″, OC(O)OR′″, CN, N(R′″)₂, NC(O)R′″, NC(O)OR′″, C(O)N(R′″)₂, NC(O)N(R′″)₂, and SR′″, wherein at each occurrence R′″ is as previously defined; (b) a saturated or unsaturated, linear, branched or cyclic C₁-C₁₂ alkyl-R wherein R is as previously defined; (c) an aromatic ring which can be further substituted at any position by R wherein R is as previously defined; or (d) a saturated or unsaturated, linear, branched or cyclic C₁-C₁₂ alkyl optionally terminated by an aromatic ring which can be further substituted as defined in (c).

In particular embodiments, resorcinol and its derivatives and/or analogs include compounds having Formula (XIII),

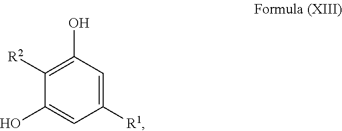

wherein:
R¹ is (a) straight or branched alkyl chain of 7 to 12 carbon atoms; (b) —O—R³, where R³ is a straight or branched alkyl chain of 5 to 9 carbon atoms, optionally substituted by one phenyl group; or (c) —(CH₂)ₙ—O—R⁴, where n is an integer from 1 to 7, and R⁴ is a straight alkyl chain of 1 to 5 carbon atoms; and
R² is a non-cyclic terpenoid including from 10 to 30 carbon atoms.

In particular embodiments, resorcinol and its derivatives and/or analogs include compounds having Formula (XIII), wherein R¹ and R² are as follows:
R¹ is a straight alkyl chain of 5 to 8 carbon atoms, optionally substituted with one methyl group; and
R² is selected from geranyl optionally substituted with one —OH, and farnesyl optionally substituted with one —OH.

In particular embodiments, resorcinol and its derivatives and/or analogs include compounds having Formula (XIII), wherein:
R¹ is (a) straight or branched alkyl chain of 7 to 12 carbon atoms; (b) —O—R³, where R³ is a straight or branched alkyl chain of 5 to 9 carbon atoms, optionally substituted by one phenyl group; or (c) —(CH₂)ₙ—O—R⁴, where n is an integer from 1 to 7, and R⁴ is a straight alkyl chain of 1 to 5 carbon atoms; and
R² is a non-cyclic terpenoid including from 10 to 30 carbon atoms; with the proviso that when R¹ is isononyl, R² is not geranyl.

In particular embodiments, resorcinol and its derivatives and/or analogs include compounds having Formula (XIII), wherein R¹ is (a) a straight or branched alkyl of 7 to 12 carbon atoms; (b) a group —O—R³, where R³ is a straight or branched alkyl of 5 to 9 carbon atoms, or a straight or branched alkyl substituted at the terminal carbon atom by a phenyl group; or (c) a group —(CH₂)ₙ—O-alkyl, where n is an integer from 1 to 7 and the alkyl group contains 1 to 5 carbon atoms.

In particular embodiments, resorcinol and its derivatives and/or analogs include compounds of Formula (XIII), wherein R² is a non-cyclic terpenoid carbon chain such as geranyl, farnesyl, and related non-cyclic terpenes and their isomers as well as other non-cyclic paraffinic or olefinic carbon chains.

In particular embodiments, resorcinol and its derivatives and/or analogs include compounds of Formula (XIII), wherein R¹ is dimethylheptyl and R² is geranyl.

In particular embodiments, dexanabinol compounds and their derivatives and/or analogs include high enantiomeric purity compounds having Formula (XIV),

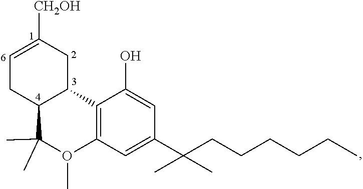

and having the (3S, 4S) configuration and being in enantiomeric excess of at least 99.90% over the (3R, 4R) enantiomer.

In particular embodiments, cannabimimetic lipid amide compounds and their derivatives and/or analogs include compounds having Formula (XV),

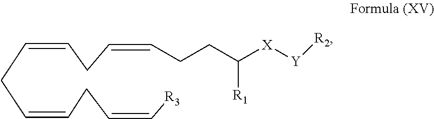

wherein:
X is one of the group consisting of C═O and NH, and Y is the other of that group. Expressed another way, X may be C═O and Y may be NH, or Y may be C═O and X may be NH, but both X and Y may not be the same group.
R₁ is H or an alkyl group. In particular embodiments, R₁ is H, CH₃, or (CH₃)₂;
R₂ is an alkyl, a substituted alkyl, an alkenyl or an alkynyl group. In particular embodiments,
R₂ is CH(R) CH₂Z, CH₂CH(R)Z, or CH(R)(CH₂)ₙCH₂Z; R being H, CH, CH₃, CHCH, CH₂CF₃, or (CH₃)₂; Z being H, halogen, N₃, NCS, or OH; and n being selected from the group consisting of 0, 1 and 2.
R₃ is an alkyl, a substituted alkyl, an aryl, an alkylaryl, an O-alkyl, an O-alkylaryl, a cyclic and a heterocyclic group. O-alkyl and O-alkylaryl refer to groups in which an oxygen atom is interposed between carbon atoms on the anandamide portion and substituent group. Examples of such R₃ groups include cyclohexyl, cyclopentyl, alkylcyclohexyl, alkylcyclopentyl, piperidinyl, morpholinyi and pyridinyl. In particular embodiments, R₃ is n-C₅H₁₀Z′, n-C₆H₁₂Z′, n-C₇H₁₄Z′, or 1′, 1′-C(CH₃)₂(CH₂)₅ CH₂Z′; Z being H, halogens, CN, N₃, NCS, or OH.

In particular embodiments, cannabimimetic lipid amide compounds and their derivatives and/or analogs include compounds having Formula (XVI),

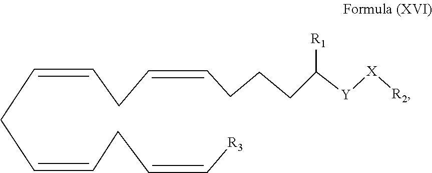

wherein:
Y is one of the group consisting of C═O and NH and X is the other of that group.
R₁ is H or an alkyl group. In particular embodiments, R₁ is H, CH₃, or (CH₃)₂.
R₂ is an alkyl, a substituted alkyl, an alkenyl, an alkynyl, an O-alkyl, a cyclic, a polycyclic, or a heterocyclic group. In particular embodiments, R₂ is

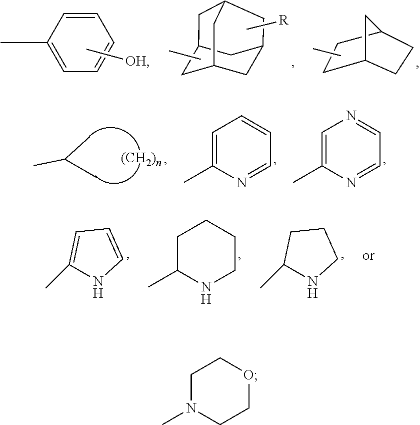

CH═CH₂, CH═C(CH₃)₂, C≡CH, CH₂OCH₃, CH(R)(CH₂)ₙCH₂Z, or CH₂CH(R)(CH₂)nZ; R being H, CH₃ or (CH₃)₂; Z being H, halogens, N₃, NCS, OH, or OAc; and n 0, 1, or 2; and
R₃ is an alkyl, a substituted alkyl, an aryl, an alkylaryl, an O-alkyl, an O-alkylaryl, a cyclic, or a heterocyclic group. In particular embodiments R₃ includes cyclohexyl, cyclopentyl, alkylcyclohexyl, alkylcyclopentyl, piperidinyl, morpholinyi and pyridinyl. In particular embodiments, R₃ is n-C₅H₁₀Z′, n-C₆H₁₂Z′, n-C₇H₁₄Z′, or 1′,1′-C(CH₃)₂CH₂)₅ CH₂Z′; Z′ being H, halogen, CN, N₃, NCS, or OH.

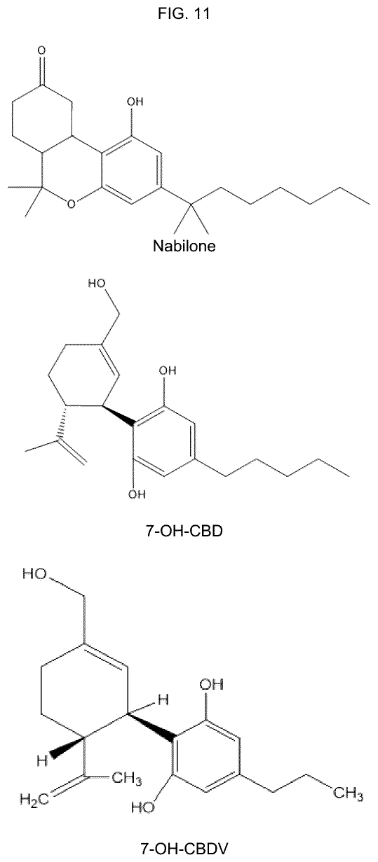

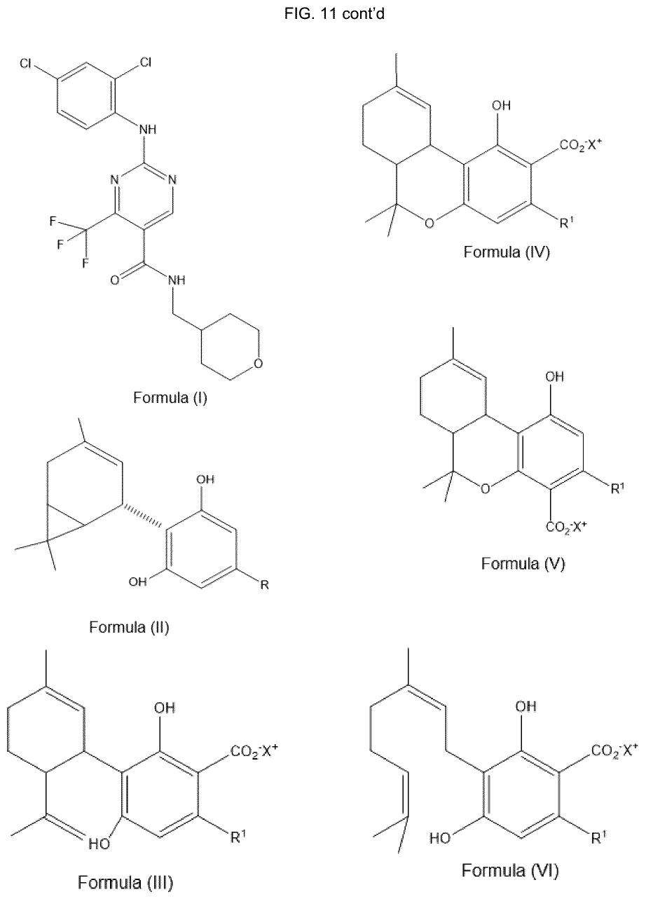

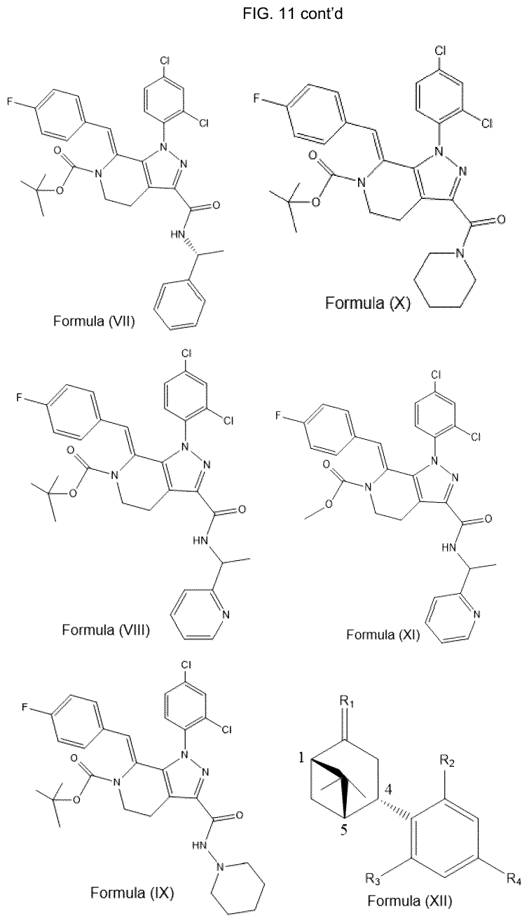

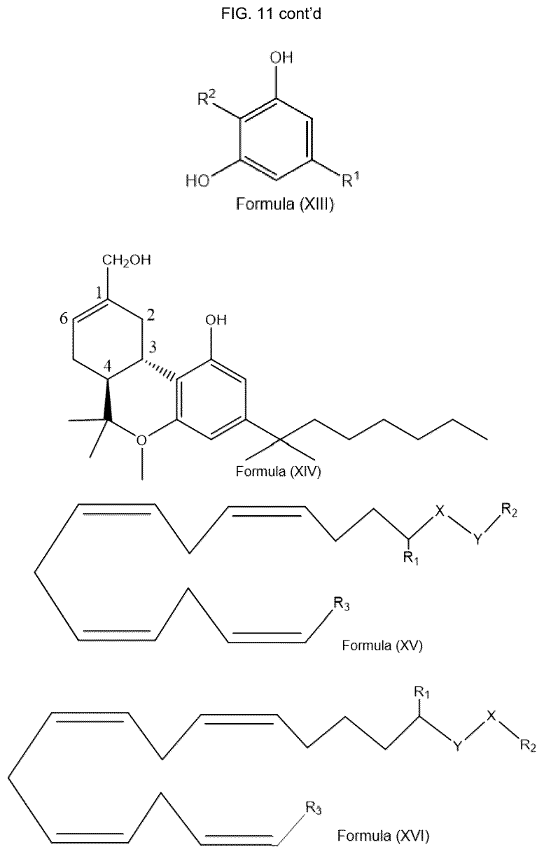

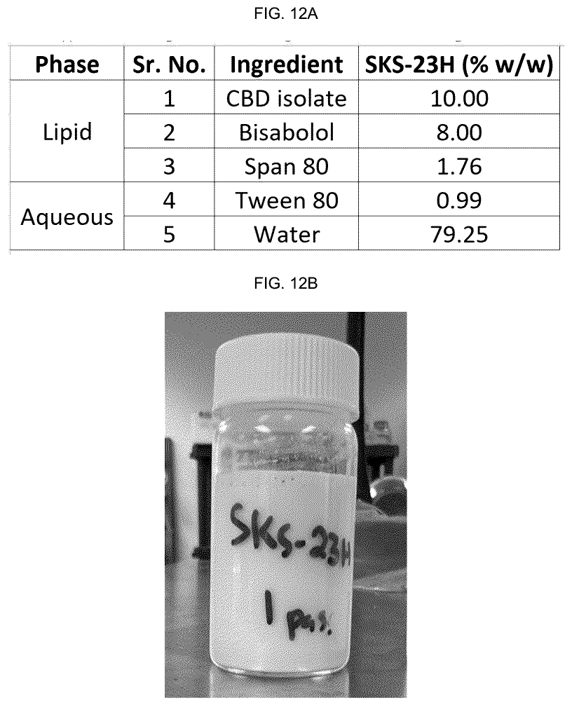

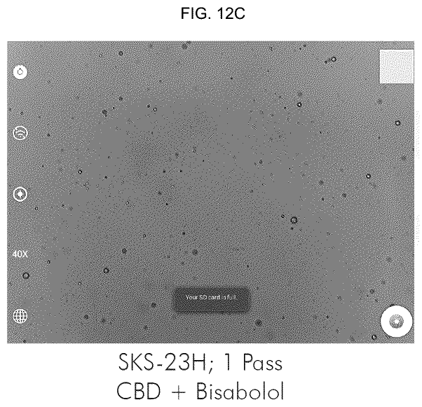

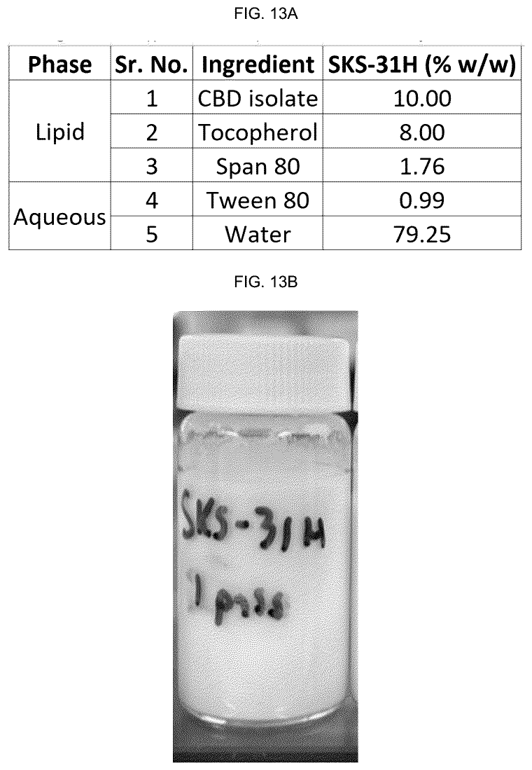

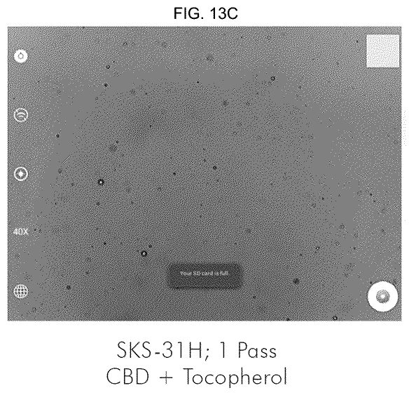

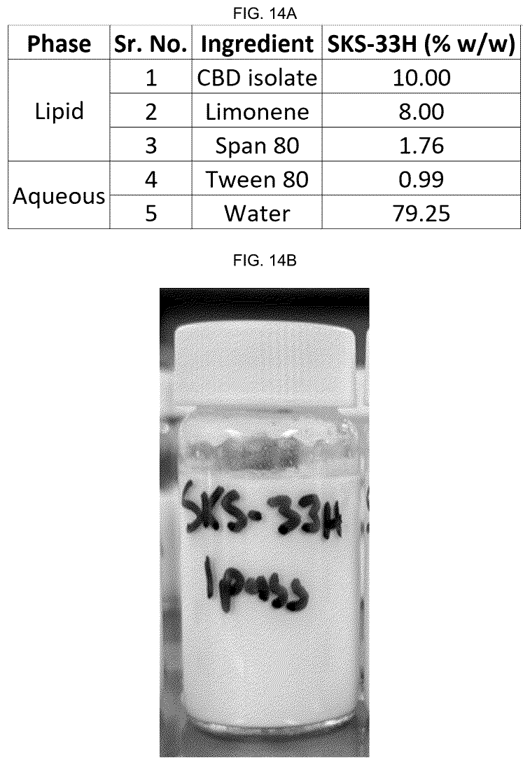

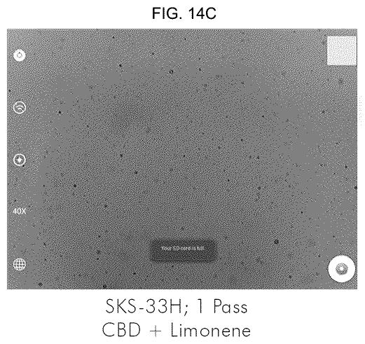

In particular embodiments, nabilone and its derivatives and/or analogs include compounds having Formula (XVII):

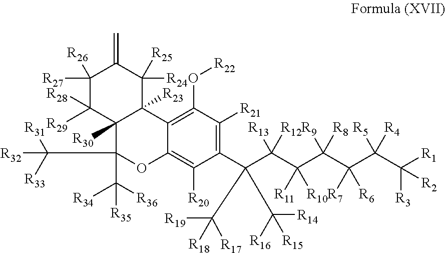

wherein:
R¹-R³⁶ are independently selected from the group consisting of hydrogen and deuterium. Nabilone derivatives and/or analogs can refer to compounds wherein at least one of R¹—R³⁶ includes deuterium. For the chemical structure of nabilone, see FIG. 11.

In particular embodiments, first LCs from the Humulus plant include: α-acids (e.g., humulone, cohumulone, adhumulone, posthumulone, prehumulone, adprehumulone) β-acids (e.g. lupulone, colupuloe, adlupulone, prelupulone, postlupulone), from a hops plant; terpenes, sesquiterpenoids, diterpenoids, and triterpenoids, phytoestrogens, and flavonoids (e.g., xanthan humulone or xanthohumol).

In particular embodiments, first LCs from a Coffea plant include: tryptophan alkaloids, diterpenes, linoleic acid, palmitic acid, triacylglycerols, diterpene esters, triterpene esters, and triterpenes.

In particular embodiments, first LCs from a Camellia plant include: resveratrol, proanthocyanidins, terpenoid esters, diacylglycerol, sterol esters, and β-amyrin esters.

In particular embodiments, first LCs from a plant include coenzyme Q10, plastoquinone, plastoquinonol, or other fat-soluble electron transport chain components found in the mitochondria of plants (and all respiring eukaryotic cells). In particular embodiments, ubiquinone and ubiquinol are forms of coenzyme Q10.

In particular embodiments, the first LC is in a lipid particle at an amount ranging from 8-95% w/w. In other examples, the first LC is in the lipid particle at an amount ranging from 32% to 40% w/w. In particular embodiments, the first LC is in the lipid particle in an amount of 10% w/w, 15% w/w, 20% w/w, 25% w/w, 30% w/w, 32% w/w, 32.5% w/w, 33% w/w, 34% w/w, 34.5% w/w, 35% w/w, 36% w/w, 36.5% w/w, 37% w/w, 38% w/w, 38.5% w/w, 39% w/w, 40% w/w, 42% w/w, 42.6% w/w, 45% w/w, 48.2% w/w, 50% w/w, 55% w/w, 60% w/w, 65% w/w, 70% w/w, 75% w/w, 80% w/w, 85% w/w, or 90% w/w. In certain embodiments, first LC is in the lipid particle at an amount of at least 32% w/w, at least 32.5% w/w, at least 33% w/w, at least 34% w/w, at least 34.5% w/w, at least 35% w/w, at least 36% w/w, at least 36.5% w/w, at least 37% w/w, at least 38% w/w, at least 38.5% w/w, at least 39% w/w %, at least 40% w/w, at least 42% w/w, at least 45% w/w, at least 48% w/w, at least 50% w/w, at least 55% w/w, at least 60% w/w, at least 65% w/w, at least 70% w/w, at least 75% w/w, at least 80% w/w, at least 85% w/w, or at least 90% w/w.

In particular embodiments, the first LC is in the lipid particle at 42.6%. In particular embodiments, the first LC is in the lipid particle at 48.2%. In particular embodiments, the first LC is in the lipid particle at 38.5%.

In particular embodiments, the first LC is CBD and is present in the lipid particle at 42.6%. In particular embodiments, the first LC is CBD and is present in the lipid particle at 48.2%. In particular embodiments, the first LC is CBD and is present in the lipid particle at 38.5%.

In particular embodiments, the first LC is in an aqueous dispersion or liquid composition at an amount ranging from 0.01% to 20% w/w. In particular embodiments, the first LC is in the aqueous dispersion or liquid composition in an amount of 0.01% w/w, 0.5% w/w, 0.75% w/w, 1% w/w, 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, 15% w/w, 16% w/w, 17% w/w, 18% w/w, 19% w/w, or 20% w/w. In certain embodiments, the first LCs is in the aqueous dispersion or liquid composition at an amount of at least 0.5% w/w, at least 0.75% w/w, at least 1% w/w, at least 2% w/w, at least 3% w/w, at least 4% w/w, at least 5% w/w, at least 6% w/w, at least 7% w/w, at least 8% w/w, at least 9% w/w, at least 10% w/w, at least 11% w/w, at least 12% w/w, at least 13% w/w, at least 14% w/w, at least 15% w/w, at least 16% w/w, at least 17% w/w, at least 18% w/w, at least 19% w/w, or at least 20% w/w.

In particular embodiments, the first LC is in the aqueous dispersion or liquid composition at 10% w/w.

In particular embodiments, the first LC is CBD and is present in the aqueous dispersion or liquid composition at 10% w/w.

Particles disclosed herein can include more than one LC. At least one of the LCs is provided as an active compound or an active ingredient. When more than one LC is used, the additional LC(s) can also be active compounds or active ingredients, and/or can be provided as carriers.

In particular embodiments, additional LCs include a second LC. In particular embodiments, additional LCs include a second LC and a third LC. In particular embodiments, additional LCs include a second LC, a third LC, and a fourth LC. In particular embodiments, additional LCs include 1, 2, 3, 4, 5, 6, 7, 8, 9, or 10 additional LCs. In particular embodiments, additional LCs include a terpene, essential oil, alkaloid, a linoleic acid, a palmitic acid, a triacylglycerol, a terpene ester, a terpenoid ester, a diacylglycerol, a sterol ester, a β-amyrin ester, a carrier oil, or any of the first LCs described earlier.

As used herein, a terpene refers to a hydrocarbon or derivative thereof, found as a natural product and biosynthesized by oligomerization of isoprene units (terpenes can also be synthetically produced). A terpene can be acyclic, monocyclic, bicyclic, or multicyclic. Examples include limonene, pulegone, caryophyllene epoxide, bisabalol and the like. As used herein, the term “terpene” includes corresponding terpenoid or sesquiterpenoid compounds. Over 100 different terpenes have been identified in the cannabis plant, and every strain tends toward a unique terpene type and composition. Examples of terpenes include: β-caryophyllene [(1R,4E,9S)-4,11,11-trimethyl-8-methylene-bicyclo(7.2.0)undec-4-ene]; s-caryophyllene oxide; citronellol [3, 7-dimethyl-Δ-octen-1-ol]; α-eudesmol [2-[(2R,4aR)-4a,8-dimethyl-2,3,4,5,6,8a-hexahydro-1H-naphthalen-2-yl]prop-an-2-ol]; β-eudesmol [2-[(2R,4aR,8aS)-4a-methyl-8-methylidene-1,2,3,4,5,6,7,8a-octahydronaphth-alen-2-yl]propan-2-ol]; gamma-eudesmol [2-[(2R,4aR)-4a, 8-dimethyl-2,3,4,5,6,7-hexahydro-1H-naphthalen-2-yl]propan-2-ol]; geraniol [(2E)-3,7-dimethylocta-2,6-dien-1-ol]; guaiol [2-[(3S,5R,8S)-3,8-dimethyl-1,2,3,4,5,6,7,8-octahydroazulen-5-yl]propan-2- -ol]; α-humulene [(1E,4E,8E)-2,6,6,9-tetramethylcycloundeca-1,4,8-triene]; β-humulene [(1E,5E)-1,4,4-trimethyl-8-methylidenecycloundeca-1, 5-diene]; gamma-humulene [(1Z,6E)-1,8, 8-trimethyl-5-methylidenecycloundeca-1,6-diene]; D-limonene [(4R)-1-methyl-4-prop-1-en-2-ylcyclohexene]; L-bmonene [(4S)-1-methyl-4-prop-1-en-2-ylcyclohexene]; (−)-linalool [(3R)-3,7-dimethylocta-1,6-dien-3-ol]; (+)-linalool [(3S)-3,7-dimethylocta-1,6-dien-3-ol]; α-myrcene [2-methyl-Δ-methybdeneocta-1,7-diene]; β-myrcene [7-methyl-3-methylideneocta-1,6-diene]; nerol [(2Z)-3,7-dimethylocta-2,6-dien-1-ol]; cis-nerolidol [(6Z)-3,7, 11-trimethyldodeca-1,6,10-trien-3-ol]; trans-nerolidol [(6E)-3,7,1 1-trimethyldodeca-1,6,10-trien-3-ol]; α-ocimene [(3E)-3,7-dimethylocta-1,3,7-triene]; ρ-ocimene [(3E)-3,7-dimethylocta-1,3,6-triene]; p-cymene [1-methyl-4-(1-methylethyl)benzene]; α-phellandrene [2-methyl-5-propan-2-ylcyclohexa-1, 3-diene]; β-phellandrene [3-methybdene-Δ-propan-2-ylcyclohexene]; cis-phytol [(Z,7R,1 1R)-3,7,1 1,15-tetramethylhexadec-2-en-1-ol]; trans-phytol [(E,7R,11R)-3,7,11,15-tetramethylhexadec-2-en-1-ol]; (−)-alphα-pinene [(1S,5S)-4,6,6-trimethylbicyclo[3.1.1]hept-3-ene]; (−)-α-pinene [(1S,5S)-6,6-dimethyl-4-methylidenebicyclo[3.1.1]heptane]; (+)-α-pinene [(1R,5R)-4,6,6-trimethylbicyclo[3.1.1]hept-3-ene]; (+)-β-pinene [(1R,5R)-6,6-dimethyl-4-methylidenebicyclo[3.1.1]heptane]; (−)-pulegone [(5S)-5-methyl-2-propan-2-yldenecyclohexan-1-one]; (+)-pulegone [(5R)-5-methyl-2-propan-2-ybdenecyclohexan-1-one]; α-terpinene [1-methyl-4-propan-2-ylcyclohexa-1, 3-diene]; Δ-terpinene [5-methyl-2-propan-2-ylcy cl ohexa-1,3-diene]; γ-terpinene [1-methyl-4-propan-2-ylcy cl ohexa-1,4-diene]; α-terpineol [2-(4-methylcyclohex-3-en-1-yl)propan-2-ol]; γ-terpineol [1-methy 1-4-propan-2-γ lidenecy clohexan-1-ol]; and (+)-valencene [(3R,4aS,5R)-4a,5-dimethyl-3-prop-1-en-2-yl-2,3,4,5,6,7-hexahydro-1H-naph-thalene].

In particular embodiments, the lipid particles include one or more terpenes selected from limonene and/or bisabolol.

In particular embodiments, terpenoids include sesquiterpenoids, diterpenoids, or triterpenoids. In particular embodiments, a sesquiterpenoid includes isohumulone. In particular embodiments, terpenes include monoterpenes, sesquiterpenes, diterpenes, or triterpenes. In particular embodiments, the monoterpene includes limonene, myrcene, geraniol, linalool, terpineol, α-pinene, or β-pinene. In particular embodiments, the sesquiterpene includes humulene, caryophyllene, or bisabolol. In particular embodiments, the triterpene includes squalene. In particular embodiments, the terpene ester includes a diterpene ester or a triterpene ester.

In particular embodiments, an essential oil includes α-pinene, β-pinene, myrcene, limonene, α-humulene, β-farnesene, β-caryophyllene, α-selinene, β-selinene, and/or γ-muurolene.

In particular embodiments, an alkaloid includes lupuline.

In particular embodiments, an additional LC includes an oil carrier. Oil carriers can include medium chain triglycerides (MCT) and/or long chain triglycerides. Oil carriers can also include borage oil, castor oil, coconut oil, cottonseed oil, soybean oil, safflower oil, sunflower oil, castor oil, corn oil, olive oil, palm oil, peanut oil, poppy seed oil, canola oil, hydrogenated soybean oil, hydrogenated vegetable oils, sesame oil, triolein, trilinolein, and trilinolenin.

In certain embodiments, the oil carrier is olive oil and/or sesame oil. In other embodiments, the oil carrier includes medium-chain triglycerides (MCT) fractionated from coconut oil, hemp oil, sunflower oil, olive oil, corn oil, or sesame oil, including mixtures thereof.

In particular embodiments, additional LCs or combinations of additional LCs are in the aqueous dispersion or liquid composition at amounts ranging from 0.01% to 20% w/w. In particular embodiments, an aqueous dispersion or liquid composition includes a terpene or a combination of terpenes in an amount of 0.01% w/w, 0.5% w/w, 0.75% w/w, 1% w/w, 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, 15% w/w, 16% w/w, 17% w/w, 18% w/w, 19% w/w, or 20% w/w. In certain embodiments, additional LCs or combinations of additional LCs are in the aqueous dispersion or liquid composition at an amount of at least 0.5% w/w, at least 0.75% w/w, at least 1% w/w, at least 2% w/w, at least 3% w/w, at least 4% w/w, at least 5% w/w, at least 6% w/w, at least 7% w/w, at least 8% w/w, at least 9% w/w, at least 10% w/w, at least 11% w/w, at least 12% w/w, at least 13% w/w, at least 14% w/w, at least 15% w/w, at least 16% w/w, at least 17% w/w, at least 18% w/w, at least 19% w/w, or at least 20% w/w.

In particular embodiments, a ratio of first LC to additional LC includes: 1:1, 1:2, 1:3, 1:4, 1:5, 1:6, 1:7, 1:8, 1:9, 1:10, 10:1, 9:1, 8:1, 7:1, 6:1, 5:1, 4:1, 3:1, 2:1, 5:4, 3:8, 9:2, 2:3, or 1:1.

In certain examples, LCs within a lipid particle are selected from the same plant type. For example, a lipid particle can include two LCs from a common plant type, three LCs from a common plant type, four LCs from a common plant type, five LCs from a common plant type, or six LCs from a common plant type. At least one of the selected LCs is solid at room temperature. These embodiments provide lipid particles with a high concentration of ingredients from a common plant type. One certain example includes CBD and humulene. Other examples include a cannabinoid (e.g., CBD) in combination with α-tocopherol, β-tocopherol, γ-tocopherol, and/or Δ-tocopherol). In these combinations, the tocopherols can provide a useful antioxidant function, preventing or reducing degradation of active compounds within a lipid particle. Other embodiments may combine particular components from different plant types, for example CBD from a cannabis plant and theanine from a tea plant.

In certain examples, the present disclosure includes an LC that is a lipophilic protein or peptide. A lipophilic protein or peptide may be an active ingredient and may be used alone or in combination with other LCs according to aspects of the present disclosure. In certain examples, the LC protein may include enzymes, such as nattokinase and bromelain that may be taken as supplements. In certain cases, the LC protein may include a membrane protein that may, for example, be used in cellular membrane targeting of lipophilic proteins. In certain examples, A lipophilic protein or peptide may be a surfactant and may be used alone or in combination with other surfactants according to aspects of the present disclosure.

(ii) Surfactants. Surfactants can be classified as nonionic, anionic, cationic, or amphoteric depending on the charge of the hydrophilic head. Nonionic surfactants are neutral and do not have any charge on their hydrophilic end. Anionic surfactants have a negative charge while cationic surfactants have a positive charge on their hydrophilic end. Amphoteric surfactants, also referred to as zwitterionic surfactants, can have either a positive, negative, or neutral net charge on their hydrophilic end depending on the pH of the environment. In acidic environments, the amphoteric surfactant behaves like a cationic surfactant and in alkaline environments, it behaves similar to an anionic surfactant.

Nonionic surfactants include sorbitan esters, polyethylene glycol sorbitan fatty acid esters, polyoxyethylene alkyl ethers, polyoxyethylene fatty acid esters, polyethylene-polypropylene glycols, saturated polyglycolized glycerides, polyethylene glycols, quillaia, polyethylene glycol stearates, polyethylene glycol glycerides, sucrose esters of fatty acids, and chemical equivalents.

Sorbitan esters include sorbitan monolaurate, sorbitan monopalmitate, sorbitan monostearate, sorbitan tristearate, sorbitan monooleate, and sorbitan trioleate.

Polyethylene glycol sorbitan fatty acid esters include polyethylene glycol sorbitan monolaurate (polysorbate 20), polyethylene glycol sorbitan monopalmitate (polysorbate 40), polyethylene glycol sorbitan monostearate (polysorbate 60), polyethylene glycol sorbitan tristearate (polysorbate 65), polyethylene glycol sorbitan monooleate (polysorbate 80), polyethylene glycol sorbitan trioleate (polysorbate 85), polyethylene glycol sorbitan hexaoleate, and polyethylene glycol sorbitan tetraoleate.

Polyoxyethylene fatty acid esters include polyoxyl 40 hydrogenated castor oil.

Polyethylene-polypropylene glycols include polyethylene-polypropylene glycol (poloxamer) 124, poloxamer 188, poloxamer 407, poloxamer 108, poloxamer 217, poloxamer 238, poloxamer 288, poloxamer 338, poloxamer 182, poloxamer 183, poloxamer 212, poloxamer 331, or poloxamer 335.

Saturated polyglycolized glycerides include lauroyl macrogol 32 glycerides.

Polyethylene glycols include polyethylene glycol (PEG) 400.

Quillaia include quillaia/quillaja saponins or quillaia extract.

Polyethylene glycol stearates include PEG 8 stearates or PEG 40 stearates.

Polyethylene glycol hydrogenated castor oils include PEG 25 hydrogenated castor oil and PEG 40 hydrogenated castor oil.

Polyethylene glycol glycerides include PEG 8 caprylic/capric glycerides or PEG 300 oleic glycerides.

Sucrose esters of fatty acids include sucrose distearate, sucrose dilaurate, sucrose palmitate.

Common anionic surfactants include dioctyl sodium sulfosuccinate (DOSS), perfluorooctanesulfonate (PFOS), linear alkylbenzene sulfonates, sodium lauryl ether sulfate, lignosulfonate, and sodium stearate.

Cationic surfactants include benzalkonium chloride (BAC), cetylpyridinium chloride (CPC), Benzethonium chloride (BZT), cetyl trimethylammonium bromide (CTAB), and cetyl trimethylammonium chloride (CTAC).

Amphoteric surfactants include lauryl betain, betaine citrate, sodium lauroamphoacetate, sodium hydroxymethylglycinate, (carboxymethyl)dimethyl-3-[(1-oxododecyl)amino] propyl ammonium hydroxide, rennin, coco alkyldimethyl betaines, (carboxymethyl) dimethyloleylammonium hydroxide, cocoamidopropyl betaine, and (carboxylatomethyl) dimethyl (octadecyl)ammonium.

In particular embodiments, the surfactant is a nonionic surfactant. In particular embodiments, the surfactant includes sorbitan monooleate. In particular embodiments, the surfactant includes polysorbate 80. In particular embodiments, the surfactant includes two or more surfactants. In particular embodiments, the surfactant includes sorbitan monooleate and polysorbate 80.

In particular embodiments, the surfactant and LC are derived from a common plant type. In particular embodiments, the common plant type is Cannabis sativa. In particular embodiments, the surfactant is hemp protein.

Surfactants can also be characterized by the hydrophilic-lipophilic balance (HLB) which is the balance of the size and strength of the hydrophilic and lipophilic moieties of a surfactant molecule ranging from 0 to 20. Lower values are more hydrophobic and higher numbers are more hydrophilic. In particular embodiments anti-foaming agents have an HLB of 1.5-3, water-in-oil emulsifiers have an HLB of 3-6, wetting agents have an HLB of 7-9, oil-in-water emulsifiers have an HLB of 8-12, and solubilizers have an HLB of 15-20. In particular embodiments, the surfactant has an HLB value greater than 4, 5, 6, 7, 8, 9, 10, 11, 12, 13, 14, 15, 16, 17, 18, 19, or 20. In other embodiments, the surfactant has an HLB value between 4-6, 5-9, 8-17, 9-16.7, 9-16.9-15, 9-14, 10-17, 10-16.7, 10-16, 10-15, 14-16, 14-17, 15-17, and between 10-14. In certain examples, the surfactant or plurality of surfactants can be chosen according to the HLB requirement of the lipid particle, aqueous dispersion, or composition.

In particular embodiments, the lipid particles include surfactant in an amount selected from: less than 20% w/w, less than 21% w/w, less than 22% w/w, less than 23% w/w, less than 24% w/w, less than 25% w/w, less than 26% w/w, less than 27% w/w, less than 28% w/w, less than 29% w/w, less than 30% w/w, less than 31% w/w, less than 32% w/w, less than 33% w/w, less than 34% w/w, and less than 35% w/w.

In particular embodiments, the aqueous dispersion or liquid composition includes surfactant in an amount selected from: less than 1% w/w, less than 2% w/w, less than 3% w/w, less than 4% w/w, less than 5% w/w, less than 6% w/w, less than 7% w/w, less than 8% w/w, less than 9% w/w, and less than 10% w/w surfactant.

(iii) Glycosides. A glycoside is any molecule in which a sugar group (the glycone) is bonded through its anomeric carbon to another group (the aglycone), which may or may not be another carbohydrate, via a glycosidic bond. Glycosides can be linked by an O— (an O-glycoside), N— (a glycosylamine), S— (a thioglycoside), or C— (a C-glycoside) glycosidic bond.

The term ‘glycoside’ is a general one which embraces all the many and varied combinations of sugars and aglycones. More precise terms are available to describe particular classes. Terms used for aglycones are generally self-explanatory (e.g., phenol, anthraquinone and sterol glycosides). The names ‘saponin’ (soap-like), ‘cyanogenetic’ (producing hydrocyanic acid) and ‘cardiac’ (having an action on the heart), although applied to these substances when little was known about them, are useful terms which do in fact bring together glycosides of similar chemical structure.

Terpene Glycosides. The flavor and sweetener agents that unexpectedly facilitate emulsification are included within the class of terpene glycosides. Rebaudiosides (REB), found naturally-occurring in Stevia rebaudiana, are steviol-type diterpene glycosides, and mogrosides (MOG), found naturally occuring in Siraitia grosvenorii, are curcurbitane-type triterpene glycosides. The structure of steviol and curcurbitane are shown below.

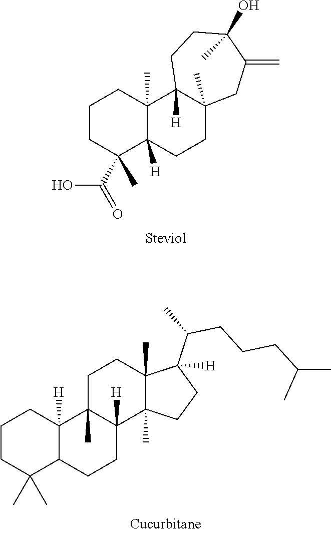

Natural terpene glycosides are well known and exist in a variety of plant sources. They generally are terpene aglycons attached to at least one glucose or other simple sugars (e.g., xylose or galactose), and the most common forms are monoterpene glycosides, diterpene glucosides, and triterpene glucosides. Many of these compounds are known to be non-toxic and natural sweeteners.

Monoterpene glycosides consist of a monoterpene (2 isoprene unit) aglycone bound to a glycone group of one, two, three, or more sugar residues. Exemplary monoterpene glycosides include paeoniflorin, albiflorin, geniposide, rosiridin, monoterpenol dihexose pentose 1, monoterpenol hexose pentose 1, monoterpenol hexose pentose 2, monoterpenol glucoside 1, monoterpenol hexose pentose 3, monoterpenol glucoside 2, monoterpenol glucoside 3, monoterpenol hexose deoxyhexose 1, monoterpenol hexose pentose 4, malonylated monoterpenol glucoside 1, monoterpenol hexose pentose 5, malonylated monoterpenol glucoside 2, monoterpenol hexose pentose 6, monoterpenol hexose pentose 7, malonylated monoterpenol glucoside 3, monoterpenol hexose pentose 8, monoterpenol hexose deoxyhexose 2, malonylated monoterpenol glucoside 4, Decyl-Beta-D-Glucopyranoside, Paeonihybridin, Paeobrin, Salicylpaeoniflorin, 6-O-β-d-Glucopyranosyl-8-O-benzoyl-paeonisuffrone, 9-Epi-paeonidanin, Paeonidanin, 3-Methyl-O-[α-arabinopyranosyl-(1→6)-β-glucopyranosyl]-6-methyl-benzofuran, (3S)-hydroxyl Terpane-10-O-β-D-glucopyranoside, (3R)-hydroxyl Terpane-10-O-β-D-glucopyranoside. Exemplary monoterpene glycosides include glycosides of limonene, geraniol, geranyl acetate, linalool, α-terpineol, α-pinene, sabinene, β-myrcene, δ-3-carene, β-pinene, α-thujene, γ-terpinene, α-terpinolene, terpinen-4-ol, α-terpinene, nerol, isomenthone, perillyl alcohol, perillic acid (perillic acid), and dihydro perillic acid.

Diterpene glycosides consist of a diterpene (4 isoprene unit) aglycone bound to a glycone group of one, two, three, or more sugar residues. Exemplary diterpene glycosides include, steviol glycosides such as rebaudioside, rebaudioside D, rebaudioside A, rebaudioside N, rebaudioside 0, rebaudioside E, stevioside, steviol monoside, steviolbioside, rubusoside, rubusoside A, dulcoside B, dulcoside A, rebaudioside B, rebaudioside G, rebaudioside C, rebaudioside F, rebaudioside I, rebaudioside H, rebaudioside L, rebaudioside K, rebaudioside J, rebaudioside M2, rebaudioside D2, rebaudioside S, rebaudioside T, rebaudioside U, rebaudioside V, rebaudioside W, rebaudioside Z1, rebaudioside Z2, rebaudioside IX, and enzymatically glucosylated steviol glycosides. The structure of rebaudioside A is shown below. Other diterpenes that contain various numbers of glucose moieties are known in the art. These compounds include: paniculoside IV, suaviosides A, B, C1, D1, D2, E, F, G, H, I, and J as identified by Ohtani et al., (1992, Phytochemistry 31(5): 1553-1559), and goshonosides F1 to F5 as identified by Seto et al., (1984, Phytochemistry 23 (12): 2829-2834).

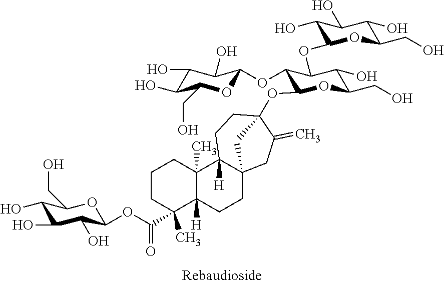

Triterpene glycosides consist of a triterpene (6 isoprene unit) aglycone bound to a glycone group of one, two, three, or more sugar residues. Exemplary triterpene glycosides include mogrosides, astragaloside, astragaloside IV, actein, cimicifugoside, cimigoside, 20-hydroxy-11-oxomogroside IAi, 11-oxomogroside NE, 11-oxomogroside IAi, mogroside ME, mogroside Ill, mogroside IV, mogroside V, siamenoside I, triterpenoid glycoside V, neogroside, Kaempferol 7-α-1-rhamnopyranoside, Kaempferol 3,7-α-1-dirhamnopyranoside, 11-oxomogroside III, 11-Dehydroxymogroside III, 11-oxomogroside IV, mogroside II, mogroside VI, 11-oxo-mogroside and siamcroside-I, 23-epi-26 deoxyactein, cimiracemoside A, 27-deoxyactein, 26-deoxycimicifugoside, acetyl shengmanol xyloside, cimicifugoside (cimigenol-3-O-β-D-Xylopyranoside), cimiaceroside A, 12β-hydroxycimigenol-3-O-β-D-xylopyranoside, 12β-hydroxycimigenol-3-O-α-L-arabinopyranoside, 21-hydroxycimigenol-3-O-α-L-arabinopyranoside, 21-hydroxycimigenol-3-O-β-D-xylopyranoside, cimigenol-3-O-α-L-arabinopyranoside, 12β-acetoxycimigenol-3-O-α-L-arabinopyranoside, 24-acetylisodahurinol-3-O-β-D-xylopyranoside, 20(S),22(R),23(S),24(R)-16β:23; 22; 25-diepoxy-12β-acetoxy-3β,23,24-trihydroxy-9,19-cycloanost-7-ene-3-O-β-D-xylopyranoside, 20(S),22(R),23(S),24(R)-16β:23; 22; 25-diepoxy-12β-acetoxy-3β,23,24-trihydroxy-9,19-cycloanost-7-en-3-O-β-L-arabinopyranoside, and 20(S),22(R),23(S),24(R)-16β:23; 22; 25-diepoxy-12β-acetoxy-3β,23,24-trihydroxy-9,19-cycloanostane-3-O-β-D-xylopyranoside. Exemplary triterpene glycosides also include glycosides of amyrin, such as α-amyrin β-amyrin and δ-amyrin. The structure of amyrin is shown below.

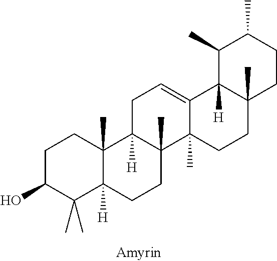

Exemplary mogrosides include grosmogroside I, mogroside IA, mogroside IE, 11-oxomogroside IA, mogroside II, mogroside II A, mogroside IIB, mogroside IIE, 7-oxomogroside IIE, mogroside III, Mogroside IIIe, 11-deoxymogroside III, mogroside IV, 11-oxomogroside IV, 11-oxomogroside IV A, mogroside V, isomogroside V, 11-deoxymogroside V, 7-oxomogroside V, 11-oxomogroside V, isomogroside V, mogroside VI, mogrol, 11-oxomogrol, siamenoside I and combinations thereof. The structure of mogroside V is shown below.

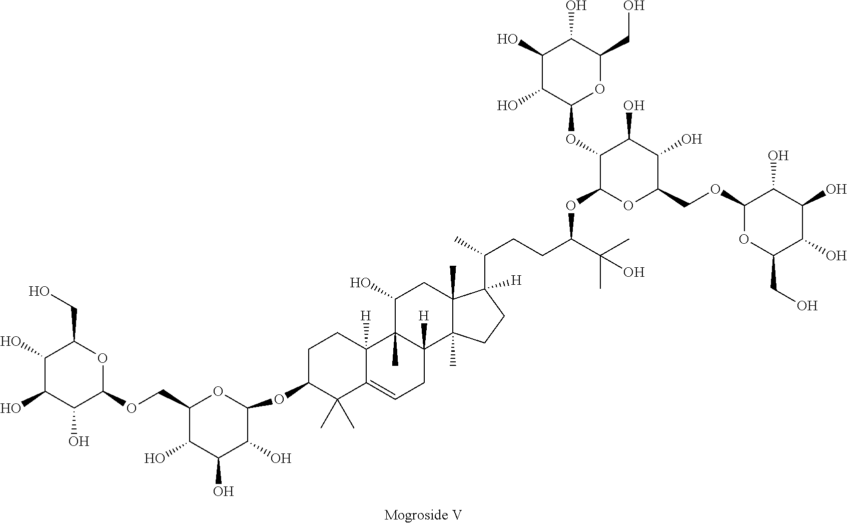

There are two major classes of terpene glycosides: monodesmosidic where there is only a single sugar chain attached at C-3 and bidesmosidic where a second sugar chain is also attached at C-24 or C-28.

Saponin Glycosides. Saponins are naturally occurring amphiphilic glycosides, which contain polar glycone structure moieties (sugars) separated from nonpolar aglycones structure moieties (also known as sapogenins). Saponin glycosides are mostly originated from plants, such as the Quillaja plant, licorice root, and Camellia plants, and tend to foam with water. Saponins are classified according to their aglycone counterparts as (i) steroidal saponins and (ii) triterpenoid saponins. The difference between these two classes is that the steroidal saponins are molecules with 27 C-atoms whereas the triterpenoid saponins are molecules with 30 C-atoms. Triterpenoid saponins are further subcategorized into (i) oleanane saponins (e.g., Sapindus mukorossi, Camellia oleifera, etc.) (ii) ursolic acid saponins (e.g., Ilex paragariensis) and (iii) dammarane saponins (e.g., Panax gingseng). Steroid saponins are also further divided into furostanol type and spirostanol type. Saponins from some families such as Solanaceae have steroidal glycoalkaloids as aglycone backbone. Based on number of sugar units, saponins are classified into (i) monodesmosidic saponins, which have a single sugar unit attached to carbon-3, (ii) bidesmosidic saponins having two sugar units attached to C-3 and C-26 or 28 and (iii) tridesmosidic saponins: a compound that consists of three sugar units attached. Branched or linear chains of sugars are attached to the aglycone. These sugar units are mostly composed of D-glucose (Glc), D-galactose (Gal), L-arabinose (Ara), L-rhamnose (Rha), D-xylose (Xyl), D-fructose (Fuc), and glucuronic acid (GIcA) [21,22]. The ginsenosides are triterpene glycosides and ginseng saponins from Panax ginseng (Chinese ginseng) and Panax quinquefolius (American ginseng). Exemplary saponin glycosides include Saponin from Quillaja, Furostan saponins, Glycyrrhizin, escin (aescin), and tea saponin.

The structure of a bidesmosidic Saponin from Quillaja is shown below.

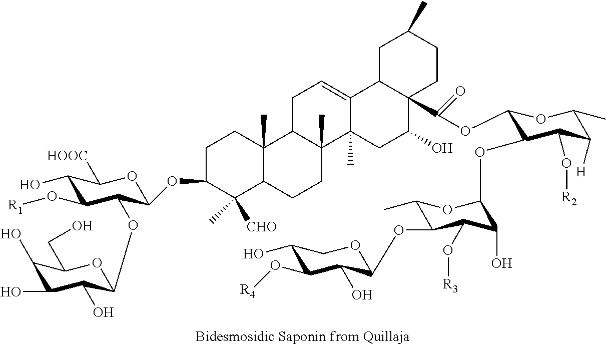

Furostan saponins of natural occurrence are bidesmosides in which a glucose monosaccharide is attached at the 26-OH group of the aglycone (with few exceptions), and an oligosaccharide chain is usually connected at 3-OH. The structure of furostan is shown below.

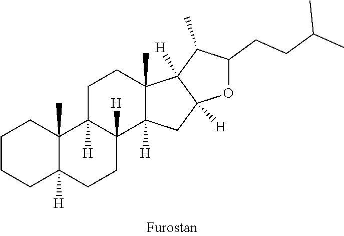

Glycyrrhizin is a triterpenoid saponin glycoside derived from licorice root, which occurs as a mixture of calcium and potassium salts of glycyrrhizic acid. The structure of glycyrrhizic acid is shown below.

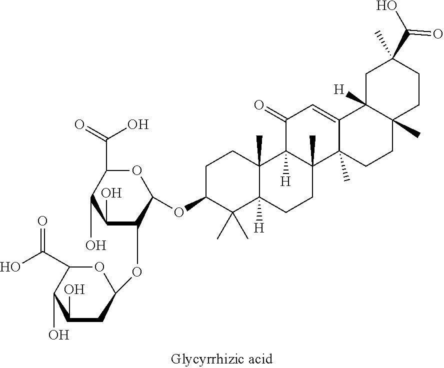

Escin or aescin is a mixture of saponins with anti-inflammatory, vasoconstrictor and vasoprotective effects found in Aesculus hippocastanum (the horse chestnut). Escin is the main active compound in horse chestnut and is responsible for most of its medicinal properties. The main active compound of escin is β-aescin, although the mixture also contains various other components including α-aescin, protoescigenin, barringtogenol, cryptoescin and benzopyrones. The structure of Escin is shown below.

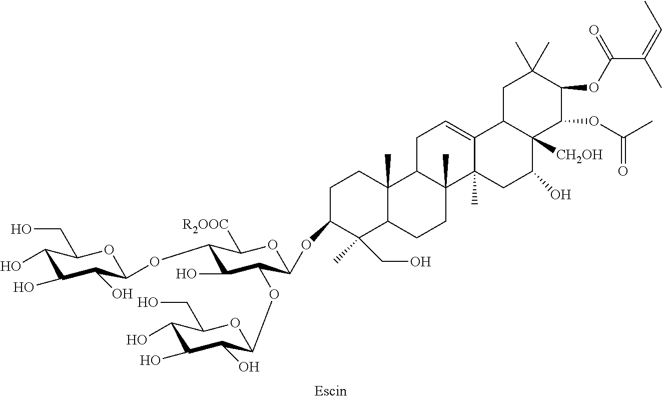

The structure of a tea saponin, found in Camellia plants, is shown below.

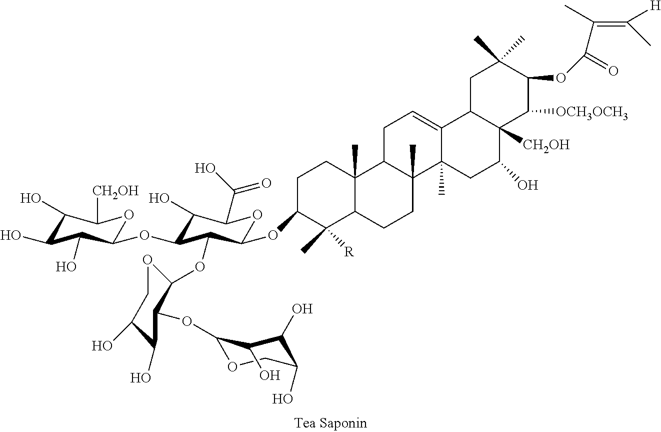

(iv) Formulations. Formulations include particles, aqueous dispersions, and compositions as disclosed herein prepared for a particular use. During preparation for a use (i.e., formulation), the nature of a lipid particle, aqueous dispersion, and/or composition may change so that it no longer meets the structural definitions or features provided above. For example, particles in an aqueous dispersion may become substantially unevenly dispersed during a formulation process. The liquid portion of an aqueous media or liquid composition may evaporate during a formulation process, such that a dry composition remains. In particular embodiments, a dry composition includes LCs, surfactant, and/or a glycoside. Other changes, such as will be understood by one of ordinary skill in the formulation arts, may also occur.

Examples of formulations include beverages and beverage kits, food products, consumer products, drops, gels, patches, films, sprays, foams, aerosols, sublingual capsules, tablets, suppositories, capsules, pessaries, tinctures, strips, lozenges, creams, lotions, balms, topicals, ointments, syrups, pastes, liquids, powders, solids, gas, inhalants, or rings, to name a few.

(v) Beverages or Beverage Kits. In particular embodiments, lipid particles, aqueous dispersions, and/or compositions of the present disclosure can be formulated into a beverage or a beverage kit. A beverage is a liquid intended for consumption that includes lipid particles, an aqueous dispersion and/or a liquid composition disclosed herein. A beverage kit includes a container including lipid particles, an aqueous dispersion, and/or a composition disclosed herein in a form to be added to a liquid for drinking. The form can be liquid, a dried powder, granules, syrups, concentrates, and the like. The kit can include the container including the lipid particles, aqueous dispersion, and/or composition, and optionally can include a beverage that the contents of the container can be added to before drinking.

Examples of beverages include coffee, tea, beer, juice, milk (both dairy and non-dairy), water, liquor, punch, a shake, soda, cocoa beverages, energy drink, drinkable yogurt, fermented beverages, cider, and wine.

In particular embodiments, beverages and/or beverage kits include flavoring agents, sweeteners, nutritional additives, preservatives, pH modifiers, coloring agents, taste-masking agents, viscosity modifiers, and thickeners.

Methods for making beverage products are described by Woodroof and Phillips, Beverages: Carbonated & Noncarbonated, AVI Publishing Co. (rev.ed. 1981); by Thorner and Herzberg, Non-alcoholic Food Service Beverage Handbook, AVI Publishing Co. (2nd ed. 1978); and by U.S. Pat. No. 4,737,375. Methods for making beverages include hot packing or aseptic packaging operations.

(vi) Food Products. In particular embodiments, lipid particles, aqueous dispersions and/or compositions of the present disclosure can be formulated into a food product. Food products generally are those that are chewed before swallowing. Examples of food products include candy (e.g., gummy candies, lollipops, mints), candy bars, bread, brownies, cakes, cheese, chocolate, cocoa, cookies, pastries, peanut butter, popcorn, protein bars, rice cakes, yogurt, sandwiches, pizza, eggs, soups, salads, salad dressings, etc.

In particular embodiments, food products are made by inclusion or infusion of the lipid particles, aqueous dispersions, and/or compositions of the present disclosure into a foodstuff. In particular embodiments, food products are made by combining lipid particles, aqueous dispersions, and/or compositions of the present disclosure with other ingredients to make an edible food product (e.g., a cookie, chocolate, lollipop, popcorn, etc.). In particular embodiments, food products are made by combining lipid particles, aqueous dispersions, and/or compositions of the present disclosure with ingredients used to make a food product, including butters and oils. Exemplary oils include coconut oil, grape seed oil, olive oil, palm oil, papaya seed oil, peanut oil, sesame oil, sprouted wheat oil, wheat germ oil, or any combination thereof.

The lipid particles, aqueous dispersions, and/or compositions of the present disclosure can be added to the food product at any stage of its preparation. For example, the lipid particles, aqueous dispersions, and/or compositions can be mixed with the food product's ingredients so that it is distributed throughout the food product and the product may be cooked, or the lipid particles, aqueous dispersions, and/or compositions can be added to the final food product. In particular embodiments, the lipid particles, aqueous dispersions, and/or compositions of the present disclosure can be applied to the surface of the food product, e.g., as a glaze, icing, or coating. In particular embodiments, the lipid particles, aqueous dispersions, and/or compositions can be sprinkled on or mixed with the food product.

In particular embodiments, food products include flavoring agents, sweeteners, nutritional additives, preservatives, pH modifiers, coloring agents, taste-masking agents, viscosity modifiers, and thickeners.

(vii) Oral Formulations. In particular embodiments, lipid particles, aqueous dispersions, and/or compositions of the present disclosure can be formulated into an oral formulation. Oral formulations referred to herein are formulations that are delivered by the mouth that are not conventional beverages or food products and are not subject to a regulatory approval process.

Examples of oral formulations include syrups, chewables, capsules, tablets, bi-layered tablets, powders, dissolvable capsules, lozenges, powders, aqueous dispersions, troches, wafers, pills, chewing gums, granules, and oils.

In particular embodiments, oral formulations can include preservatives, pH modifiers, coloring agents, taste-masking agents, viscosity modifiers, and thickeners.

Oral formulations can be manufactured using processes that are well known to those of skill in the art. For example, for the manufacture of bi-layered tablets, the agents can be dispersed uniformly in one or more excipients, for example, using high shear granulation, low shear granulation, fluid bed granulation, or by blending for direct compression. Excipients include diluents, binders, disintegrants, dispersants, lubricants, glidants, stabilizers, surfactants and colorants. Diluents, also termed “fillers”, can be used to increase the bulk of a tablet so that a practical size is provided for compression. Examples of diluents include lactose, cellulose, microcrystalline cellulose, mannitol, dry starch, hydrolyzed starches, powdered sugar, talc, sodium chloride, silicon dioxide, titanium oxide, dicalcium phosphate dihydrate, calcium sulfate, calcium carbonate, alumina and kaolin. Binders can impart cohesive qualities to a tablet formulation and can be used to help a tablet remain intact after compression. Non-limiting examples of suitable binders include starch (including corn starch and pregelatinized starch), gelatin, sugars (e.g., glucose, dextrose, sucrose, lactose and sorbitol), celluloses, polyethylene glycol, waxes, natural and synthetic gums, e.g., acacia, tragacanth, sodium alginate, and synthetic polymers such as polymethacrylates and polyvinylpyrrolidone. Lubricants can also facilitate tablet manufacture; examples thereof include magnesium stearate, calcium stearate, stearic acid, glyceryl behenate, and polyethylene glycol. Disintegrants can facilitate tablet disintegration after administration, and examples thereof include starches, alginic acid, crosslinked polymers such as, e.g., crosslinked polyvinylpyrrolidone, croscarmellose sodium, potassium or sodium starch glycolate, clays, celluloses, starches, gums and the like. Examples of suitable glidants include silicon dioxide, talc and the like. Stabilizers can inhibit or retard drug decomposition reactions, including oxidative reactions. Surfactants can also include and can be anionic, cationic, amphoteric or nonionic. If desired, the tablets can also include nontoxic auxiliary substances such as pH buffering agents, preservatives, e.g., antioxidants, wetting or emulsifying agents, solubilizing agents, coating agents, flavoring agents, and the like.

Controlled-release formulations can include one or more combinations of excipients that slow the release of the agents by coating or temporarily bonding or decreasing the solubility of the active agents. Examples of these excipients include cellulose ethers such as hydroxypropylmethylcellulose (e.g., Methocel K4M) or silicified microcrystalline cellulose, polyvinylacetate-based excipients such as, e.g., Kollidon SR, and polymers and copolymers based on methacrylates and methacrylic acid such as, e.g., Eudragit NE 30D.

Immediate-release formulations can include one or more combinations of excipients that allow for a rapid release of an active compound or active ingredient (such as from 1 minute to 1 hour after administration). In one embodiment an immediate release excipient can be microcrystalline cellulose, sodium carboxymethyl cellulose, sodium starch glycolate, corn starch, colloidal silica, Sodium Laurel Sulphate, Magnesium Stearate, Prosolve SMCC(HD90), croscarmellose Sodium, Crospovidone NF, Avicel PH200, and combinations of such excipients.

In particular embodiments, the formulations may be formulated as extended release, sustained release, or as a long acting tablet or capsule. Methods of making sustained release tablets are known in the art; see e.g., U.S. Patent Publication No. 2006/0051416 to Rastogi, and U.S. Patent Publication No. 2007/0065512 to Dedhiya, which are hereby incorporated by reference in their entirety. Gradual-release tablets are also known in the art; examples of such tablets are set forth in U.S. Pat. No. 3,456,049 to Hotko, which is hereby incorporated by reference in its entirety. A slow- or sustained-release formulation may delay disintegration or absorption of the formulation or one or more components thereof.

Oral formulations can include additional active compounds or active ingredients and can be formulated with any compounds suitable for administration as an oral formulation. In particular embodiments, the oral formulation can include components that do not impair the desired action of the oral formulation and/or components that supplement the desired action of the oral formulation.

In particular embodiments, the oral formulation is a syrup. In particular embodiments, the first LC concentration in the syrup is 0.05 mg/ml to 100 mg/ml, 0.5 mg/ml to 10 mg/ml. In particular embodiments, the first LC concentration in the syrup is 1 mg/ml. In particular embodiments, the first LC in the syrup is 2.5 mg/ml to about 50 mg/ml.

Oral formulations in the form of syrups or other liquid formulations can be incorporated into an acceptable single-dose or multi-dose container for oral dosing of the formulation.

(viii) Mucosal Formulations. In particular embodiments, lipid particles, aqueous dispersions, and/or compositions of the present disclosure can be formulated into a mucosal formulation.

Examples of mucosal formulations include nebulizers, ointments, creams, lotions, foams, sprays, mousses, patches, powders, pastes, gels, tablets, liquid droplets, suppositories, and dissolvable films. Mucosal formulations referred to herein refer to formulations delivered through moist cavities such as the lining of the mouth (sublingual and buccal), bladder, rectal, genital, or nasal cavity. Mucosal formulations administered buccally can be formulated into lozenges, sprays, gels, pastes, dissolvable tablets or dissolvable strips. Mucosal formulations administered sublingually can be formulated into lozenges, sprays, gels, pastes, dissolvable tablets or dissolvable strips. Mucosal formulations administered bronchially or pulmonary can be formulated into an aerosol, pressurized atomizers, inhalers of dry powder, or dissolved in volatile liquids. Mucosal formulations administered rectally can be formulated into suppositories, ointments, creams, suspensions, solutions, lotions, pastes, gels, sprays, foams or oils. Mucosal formulations administered vaginally can be formulated into suppositories, ointments, creams, suspensions, solutions, lotions, pastes, gels, sprays, foams or oils. Mucosal formulations administered nasally can be formulated into ointments, suspensions, solutions, lotions, pastes, gels, sprays or mists.

In particular embodiments, mucosal formulations can include preservatives, pH modifiers, coloring agents, taste-masking agents, film-forming polymers, penetration enhancers, viscosity modifiers, lubricants, and thickeners.

In particular embodiments, mucosal formulations include liquid droplets administered sublingually. In particular embodiments, mucosal formulations include nebulizers for administration nasally. In particular embodiments, mucosal formulations include a spray for administration nasally. In particular embodiments, mucosal formulations include a suppository.

In particular embodiments, mucosal formulations include a nebulizer. Nebulizers are broadly known to those of skill in the art and the invention is not limited to any specific type of nebulizer. Examples of suitable nebulizers and/or delivery devices are described in: U.S. Pat. Nos. 7,036,500; 7,029,656; 7,013,894; 6,994,083; 6,962,151; 6,929,003; 6,854,662; 6,748,945; 6,732,731; 6,729,327; 6,598,602; 5,853,002; 5,549,102; 5,435,282; 5,036,840; 7,077,126; 7,059,320; 6,983,747; 6,679,251; 6,606,990; 6,514,177; 513,727; 6,513,519; 6,464,388; 6,176,237; 6,085,741; 6,000,394; 5,957,389; 5,740,966; 5,596,982; 5,461,695; 5,458,136; 5,312,046; 5,309,900; 5,280,784; and U.S. Publication Nos.: 20060102172; 20060065267; 20060054166; 20060048772; 20060011196; 20050224076; 20050056274; 20050039741; 20040250816; 20030037788; 20030037785; 20020005196 and 20010054421 and the like. The disclosures of which are incorporated by reference in their entireties.

(ix) Consumer Products. In particular embodiments, lipid particles, aqueous dispersions, and/or compositions of the present disclosure can be formulated onto a consumer product such that the particles or their remnants are found on or near the surface of the consumer product. In certain examples, the addition of a liquid to the surface of the consumer product results in the release of the lipid particles or their remnants into the liquid.

In particular embodiments, a consumer product includes any product that comes into contact with an ingestible liquid or other ingestible or inhalable substrate. Examples of such products include liquid-holding receptacles (e.g., cups or bowls), utensils (e.g., spoons, forks, knives), peripheral cooking objects (e.g., coffee filters), wrappers (e.g., food or tobacco wrappers), etc. The consumer product can be made of glass, paper, plastic, ceramics, stoneware, styrofoam, cardboard, metal, or combinations thereof.

In particular embodiments, the particles, aqueous dispersions, and/or compositions can be applied to the surface of or incorporated into the consumer product by any method known in the art including by printing, solvent-based methods, adsorption, absorption, heat-based methods, and/or mechanochemical activation-based methods. In particular embodiments, the lipid particles, aqueous dispersions, and/or compositions can be applied to the surface of a consumer product by spray drying, film deposition, extrusion-based printing, dip coating, ink-jet printing, bead coating, or any other method known in the art. The lipid particles, aqueous dispersions, and/or compositions can be deposited on the surface of the consumer product in any shape or orientation desired including stripes, amorphous blobs, circles, words, or logos. In particular embodiments, a colorant is included in the aqueous dispersions, and/or compositions such that the human eye can see the shape and/or orientation of the formulation on the consumer product. Once the formulation on or within the consumer product comes into contact with a liquid or other medium (e.g., heated air), the formulation can be released from the consumer product surface such that it will be able to be dispersed ingested or inhaled.

In particular embodiments, coating formulations on consumer products can further include mechanical modifiers, plasticizers, disintegrants, film-forming polymers, thinners, thickeners, preservatives, pH modifiers, taste-masking agents, viscosity modifiers, and/or lubricants.

Mechanical modifiers are used to improve the processing flowability and physical properties of a formulation and can include magnesium stearate, stearic acid, sodium stearyl fumarate, hydrogenated vegetable oil, polyethylene glycol, sugars, mannitol, sorbitol, lactose, whey and yeast, microcrystalline cellulose, povidone, carbopol, crospovidone, croscarmellose sodium, gellan gum, silicone dioxide, pectin, gelatin, carrageenan, carnauba wax, carob gum, agar, mannan, sodium alginate, and shellac.

In particular embodiments, edible plasticizers include glycerol, triacetine, sorbitan, corn starch and citric acids, guar gum, and propylene glycol.

An exemplary disintegrant includes a milled lactose powder which can allow the formulation to release an LC at a desired rate.

In particular embodiments, a thinner includes food grade ethanol, other alcohol, or water. In particular embodiments, thickeners include starch. Exemplary starches include pea starch, potato starch, corn starch, microcrystalline cellulose, wheat starch, rice starch, and combinations thereof.

(x) Common Considerations & Components for Drinkable, Ingestible, and/or Inhalable Products & Formulations. Flavoring agents useful herein include any material or mixture of materials operable to enhance the taste of a drinkable formulation. Any orally acceptable natural or synthetic flavoring agents can be used, such as essential oils, various flavoring aldehydes, flavoring oils, esters, alcohols, similar materials, as well as sweeteners such as sodium saccharin, xylitol, D-mannose, and combinations thereof. Essential oils can include: Ylang Ylang (Cananga odorata); Yarrow (Achillea millefolium); Violet (Viola odorata); Vetiver (Vetiveria zizanoides); Vanilla (Vanilla plantifolia); Tuberose (Polianthes tuberosa); Thyme (Thymus vulgarisL.); Tea Tree (Melaleuca altemifolia); Tangerine (Citrus reticulata); Spruce, Black (Picea mariana); Spruce (Tsuga Canadensis); Spikenard (Nardostachys jatamansi); Spearmint (Mentha spicata); Sandalwood (Santalum spicatum); Rosewood (Aniba rosaeodora); Rosemary Verbenone (Rosmarinus officinalis); Rosemary (Rosmarinus officinalis); Rose (Rosa damascena); Rose Geranium (Pelargonium roseum); Ravensara (Ravensara aromatica); Plai (Zingiber cassumunar) Pine Needle (Pinus sylvestrisL.); Petitgrain (Citrus aurantium); Peppermint (Mentha piperita); Pepper, Black (Piper nigrumL.); Patchouli (Pogostemon cablin); Palo Santo (Bursera graveolens); Palmarosa (Cymbopogon martini); Osmanthus (Osmanthus fragrans); Oregano (Origanum vulgare); Orange, Sweet (Citrus sinensis); Oak Moss (Evemia prunastri); Nutmeg (Myristica fragrans) Niaouli (Melaleuca viridifloria); Neroli (aka Orange Blossom) (Citrus aurantium); Myrtle (Myrtus communis); Myrrh (Commiphora myrrha); Mimosa (Acacia decurrens); Melissa (Melissa officinalis L); Marjoram, Sweet (Origanum majorana); Manuka (Leptospermum scoparium); Mandarin, Red (Citrus deliciosa); Mandarin (Citrus deliciosa); Lotus, White (Nelumbo nucifera); Lotus, Pink (Nelumbo nucifera); Lotus, Blue (Nelumbo nucifera); Lime (Citms aurantifolia); Lily (Lilum aurantum); Lemongrass (Cymbopogon citratus); Lemon (Citrus limonum); Lavender (Lavandula angustifolium); Lavandin (Lavandula hybrida grosso); Kanuka (Kunzea ericoides); Juniper Berry (Juniperus cummunis); Jasmine (Jasminum officinale); Jasmine Abs (Jasminum sambac); Helichr sum (Helichrysum italicum); Grapefruit, White (Citrus x paradisi); Grapefruit, Pink (Citrus paradisi); Ginger (Zingiber officinalis); Geranium (Pelargonium graveolens); Geranium, Bourbon (Pelargonium graveolens, 'Herit); Gardenia (Gardenia jasminoides); Galbanum (Ferula galbaniflua); Frankincense (Boswellia carterii); Frangipani (Plumeria alba); Fir Needle White (Abies alba); Fir Needle Siberia (Abies sibenca); Fir Needle Canada (Abies balsamea); Fennel, Sweet (Foeniculum vulgare); Eucalyptus Smithii. Eucalyptus Radiata, Eucalyptus Globulus, Eucalyptus Citriodora, Eucalyptus Blue Mallee (Eucalyptus polybractea); Elemi (Canarium luzonicum); Dill (Anethum graveolens); Cypress (Cupressus sempervirens); Cumin (Cuminum cyminum); Coriander (Coriandum sativum); Cocoa (Theobroma cacao); Clove (Eugenia caryophylatta); Clary Sage (Salvia sclarea); Cistus (aka Labdanum) (Cistus ladaniferusL.); Cinnamon (Cinnamomum zeylanicum); Chamomile, Roman (Anthemis nobilis); Chamomile, Blue (Matricaria chamomilla); Celery Seed (Apium graveolins); Cedarwood, Western Red (Thuja plicata); Cedarwood, Blood (Juniperus virginiana); Cedarwood Atlas (Cedrus atlantica); Carrot Seed (Daucus carota); Cardamon (Elettaria cardamomum); Caraway Seed (Carum carvi); Cajeput (Melaleuca cajuputi); Cade (Juniperus oxy cedrus); Birch, White (Betula alba); Birch, Sweet (Betula lenta); Bergamot (Citrus bergamia); Bay Laurel (Lauras nobilis); Basil (Ocimum basilicum); Basil, Holy (Ocimum sanctum); Basil (Ocimum basilicum); Balsam Poplar (Populus balsamifera); Balsam Peru (Myroxylon balsamum); Angelica (Angelica archangelicaL.); and combinations thereof. Flavoring agents include, for example, vanillin, sage, marjoram, parsley oil, spearmint oil, cinnamon oil, oil of wintergreen (methylsalicylate), peppermint oil, clove oil, bay oil, anise oil, eucalyptus oil, citrus oils, fruit oils, and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavoring agents, and mixtures thereof. Flavoring agents also include ingredients that provide fragrance and/or other sensory effect in the mouth, including cooling or warming effects. Such ingredients include menthol, menthyl acetate, menthyl lactate, camphor, eucalyptus oil, eucalyptol, anethole, eugenol, cassia, oxanone, [α]-irisone, propenyl guaiethol, thymol, linalool, benzaldehyde, cinnamaldehyde, N-ethyl-p-menthan-3-carboxamme, N,2,3-trimethyl-2-isopropylbutanamide, 3-1-menthoxypropane-1,2-diol, cinnamaldehyde glycerol acetal (CGA), methane glycerol acetal (MGA) and mixtures thereof. In particular embodiments, the formulation includes a lipophilic flavoring agent including menthol, vanillin or an essential oil (e.g., orange oil, lemon oil, clove oil, peppermint oil, spearmint oil or aniseed oil). In particular embodiments, flavoring agents can be present in the formulation at amounts ranging from 1 to 10 mg/ml, 10 to 50 mg/ml, 50 to 100 mg/ml, 100 to 250 mg/ml, 250 to 500 mg/ml, 500 to 750 mg/ml, or 750 to 1000 mg/ml.

In particular embodiments, flavoring agents include lemon juice concentrate, tangerine flavor, tangerine concentrate, and/or apple juice concentrate. In particular embodiments, flavoring agents include jasmine, rooibos, and/or peppermint tea.

Sweeteners include maltose, sucrose, glucose, fructose, invert sugars and mixtures thereof. These sugars can be incorporated into the aqueous dispersion in solid or liquid form. Fructose can be obtained or provided as liquid fructose, high fructose corn syrup (HFCS), dry fructose or fructose syrup. High fructose corn syrup is commercially available as HFCS-42, HFCS-55 and HFCS-90, which include 42%, 55% and 90%, respectively, by weight of the sugar solids therein of fructose. In particular embodiments, sweeteners can be provided to some extent by other components of the aqueous dispersion, such as by fruit juice, flavorants, and so forth. Sweeteners can be employed in the process of the present disclosure in amounts ranging from 0.01% to 20%, from 6% to 14%, sugar solids by weight of the aqueous dispersion.

Optional artificial or noncaloric sweeteners can be used alone or in combination with carbohydrate sweeteners in beverages or beverage kits. They include, for example, saccharin, cyclamates, acetosulfam, L-aspartyl-L-phenyalanine lower alkyl ester sweeteners (e.g., aspartame). L-aspartyl-D-alanine amides disclosed in U.S. Pat. No. 4,411,930 to Brennan et al., L-aspartyl-D-serine amides disclosed in U.S. Pat. No. 4,399,163 to Brennan et al., L-aspartyl-L-1-hydroxymethyl-alkaneamide sweeteners disclosed in U.S. Pat. No. 4,338,346 to Brand, L-aspartyl-1-hydroxyethylakaneamide sweeteners disclosed in U.S. Pat. No. 4,423,029 to Rizzi, L-aspartyl-D-phenylglycine ester and amide sweeteners disclosed in European Patent Application 168,112 to J. M. Janusz, published Jan. 15, 1986, and the like. Artificial or noncaloric sweeteners, if used, are typically employed in an amount ranging from 0.01% to 1%, from 0.05% to 0.10% by weight of the beverage portion of the beverage or beverage kit.

Nutritional additives include essential oils, vitamins, minerals, and amino acids. Vitamins include Vitamin A, vitamin B1, vitamin B2, vitamin B3, vitamin B12, vitamin C, vitamin E, vitamin D, niacin (vitamin PP), biotin (vitamin H), menadione (vitamin K), folic acid, and pyridoxine (B6). Minerals include iron, zinc, calcium, phosphorus, potassium, magnesium, and fluoride. Amino acids include lysine, histidine, isoleucine, leucine, methionine, phenylalanine, threonine, tryptophan, and valine. Exemplary nutritional additives can be found in Roberts et al., Nutraceuticals: The Complete Encyclopedia of Supplements, Herbs, Vitamins, and Healing Foods (American Nutraceutical Association, 2001); in Physicians' Desk Reference for Nutritional Supplements, 1st Ed. (2001); and in The Physicians' Desk Reference for Herbal Medicines, 1st Ed. (2001), which are incorporated by reference.

Taste-masking agents include sodium bicarbonate, ion-exchange resins, cyclodextrin inclusion compounds, adsorbates, and the like.

(xi) Ocular Formulations. In particular embodiments, lipid particles, aqueous dispersions, and/or compositions of the present disclosure can be formulated into an ocular formulation.

Ocular formulations include aqueous dispersions, suspensions (e.g., eye drops), viscous or semi-viscous gels, or other types of solid or semisolid compositions (e.g., ointments) or sustained release devices or mechanisms that are placed in or around the eye. Examples of ocular formulations include eye drops and ointments.

In particular embodiments, an ointment is an oil-based or oil-and-water-based semi-solid or viscous formulation with a melting or softening point near body temperature. In particular embodiments, eye drops are a liquid formulation that can be applied to the eye as droplets. In particular embodiments, a gel is a jelly-like viscous formulation that includes a matrix of interacting molecules that confer viscosity, and a liquid (e.g., suspension) that is dispersed within the matrix.

Ocularly administrable formulations can also be provided with tear substitutes. “Tear substitutes” refer to molecules or compositions which lubricate or “wet,” approximate the consistency of endogenous tears, aid in natural tear build-up, or otherwise provide temporary relief of eyes upon ocular administration. A variety of tear substitutes are known in the art and include: monomeric polyols, such as, glycerol, propylene glycol, and ethylene glycol; polymeric polyols such as polyethylene glycol; cellulose esters such hydroxypropylmethyl cellulose, carboxymethyl cellulose sodium and hydroxy propylcellulose; dextrans such as dextran 70; water soluble proteins such as gelatin; vinyl polymers, such as polyvinyl alcohol, polyvinylpyrrolidone, and povidone; and carbomers, such as carbomer 934P, carbomer 941, carbomer 940 and carbomer 974P. Many such tear substitutes are commercially available, which include cellulose esters such as Bion® Tears (Alcon Research, Ltd., Fort Worth, Texas, U.S.A.), Celluvisc® (Allergan, Inc., Irvine, California, U.S.A.), Genteal® (Novartis Pharmaceuticals Corporation, East Hanover, New Jersey, U.S.A.), OccuCoat® (Barnes-Hind, Inc. Clearwater, Florida, U.S.A.), Refresh® (Allergan, Inc., Irvine, California, U.S.A.), Systane® (Novartis AG, Basel, Switzerland), Systane Ultra® (Novartis AG, Basel, Switzerland), Refresh Endura® (Allergan, Inc., Irvine, California, U.S.A.), Refresh Liquigel® (Allergan, Inc., Irvine, California, U.S.A.), Teargen II™ (McKesson), Tears Naturale® (Alcon (Puerto Rico), Inc., Humacao, Puerto Rico), Tears Naturale® II (Alcon (Puerto Rico), Inc., Humacao, Puerto Rico), Tears Naturale Free® (Alcon (Puerto Rico), Inc., Fort Worth, Texas, U.S.A.), and TheraTears® (Advanced Vision Research, Inc., Ann Arbor, Michigan, U.S.A.); and polyvinyl alcohols such as Akwa Tears® (Akorn, Inc., Lake Forest, Illinois, U.S.A.), HypoTears® (Novartis Pharmaceuticals Corporation, East Hanover, New Jersey, U.S.A.), Moisture Eyes® (Bausch & Lomb Incorporated, Rochester, New York, U.S.A.), Murine Tears® (Medtech Products, Inc., Irvington, New York, U.S.A.), Visine Tears® (Johnson & Johnson, New Brunswick, New Jersey, U.S.A.), and Soothe® (Bausch & Lomb Incorporated, Rochester, New York, U.S.A.). Tear substitutes may also include paraffins, such as the commercially available Lacri-Lube® (Allergan, Inc., Irvine, California, U.S.A.) ointments. Other commercially available ointments that are used as tear substitutes include Lubrifresh PM™ (Bausch & Lomb Incorporated), Moisture Eyes® PM (Bausch & Lomb Incorporated, Rochester, New York, U.S.A.) and Refresh P.M.® (Allergan, Inc., Irvine, California, U.S.A.).

Additional potential excipients for ocularly administrable formulations include solubilizing agents, stabilizing agents, surfactants, demulcents, viscosity modifiers, diluents, inert carriers, preservatives, binders, and/or disintegrants. Further examples of excipients include certain inert proteins such as albumins; hydrophilic polymers such as polyvinylpyrrolidone; amino acids such as aspartic acid (which may alternatively be referred to as aspartate), glutamic acid (which may alternatively be referred to as glutamate), lysine, arginine, glycine, and histidine; fatty acids and phospholipids such as alkyl sulfonates and caprylate; surfactants such as sodium dodecyl sulphate and polysorbate; nonionic surfactants such as such as TWEEN® (Tween-Croda Americas, LLC, Wilmington, DE, PLURONICS® (Pluronic-BASF Corp., Mount Olive, NJ), or a polyethylene glycol (PEG) designated 200, 300, 400, or 600; a Carbowax designated 1000, 1500, 4000, 6000, and 10000; carbohydrates such as glucose, sucrose, mannose, maltose, trehalose, and dextrins, including cyclodextrins; polyols such as mannitol and sorbitol; chelating agents such as EDTA; and salt-forming counter-ions such as sodium.

Eye drops may be prepared by dispersing lipid particles into a sterile aqueous solution or aqueous dispersion such as physiological saline, buffering solution, etc., or by combining powder compositions to be dissolved before use. Other vehicles may be chosen, as is known in the art: balance salt solution, saline solution, water soluble polyethers such as polyethylene glycol, polyvinyls, such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate. If desired, additives ordinarily used in the eye drops can be added. Such additives include isotonizing agents (e.g., sodium chloride, etc.), buffer agent (e.g., boric acid, sodium monohydrogen phosphate, sodium dihydrogen phosphate, etc.), preservatives (e.g., benzalkonium chloride, benzethonium chloride, chlorobutanol, etc.), thickeners (e.g., saccharide such as lactose, mannitol, maltose, etc.; e.g., hyaluronic acid or its salt such as sodium hyaluronate, potassium hyaluronate, etc.; e.g., mucopolysaccharide such as chondroitin sulfate, etc.; e.g., sodium polyacrylate, carboxyvinyl polymer, crosslinked polyacrylate, polyvinyl alcohol, polyvinyl pyrrolidone, methyl cellulose, hydroxy propyl methylcellulose, hydroxyethyl cellulose, carboxymethyl cellulose, hydroxy propyl cellulose or other agents known to those skilled in the art).

For eye drops, the formulations can include concentrations of the first LC ranging from 0.0001 to 10.0 w/v %, 0.005 to 10.0 w/v %, 0.01 to 10.0 w/v %, 0.05 to 10.0 w/v %, 0.1 to 10.0 w/v %, 0.5 to 10.0 w/v %, 1.0 to 10.0 w/v %, 20 to 10.0 w/v %, 3.0 to 10.0 w/v %, 4.0 to 10.0 w/v %, or 5.0 to 10.0 w/v %. The administration may be administered several times a day per eye, one to ten times, one to four times, or once a day. The size of the drop administered may be in the range of 10-100 μl, 10-90 μl, 10-80 μl, 10-70 μl, 10-60 μl, 10-50 μl, 10-40 μl, 10-30 μl, 20-100 μl, 20-90 μl, 20-80 μl, 20-70 μl, 20-60 μl, 20-50 μl, 20-40 μl, or 20-30 μl.

(xii) Topical Formulations. In particular embodiments, lipid particles, aqueous dispersions, and/or compositions of the present disclosure can be formulated into a topical formulation. Topical formulations are formulations that are applied directly to a body surface, such as skin. Topical formulations can be in the form of foams, sprays, mousses, patches, powders, pastes, medicated plasters, creams, ointments, lotions, or gels.

Penetration enhancers increase dermal penetration and include 2-(2-ethoxyethoxy)ethanol and dimethyl isosorbide in the range of 5-30% (w/w). Other non-limiting examples of penetration enhancers include C8-C22 fatty acids such as isostearic acid, octanoic acid, and oleic acid; C8-C22 fatty alcohols such as oleyl alcohol and lauryl alcohol; lower alkyl esters of C8-C22 fatty acids such as ethyl oleate, isopropyl myristate, butyl stearate, and methyl laurate; di(lower)alkyl esters of C6-C8 diacids such as diisopropyl adipate; monoglycerides of C8-C22 fatty acids such as glyceryl monolaurate; tetrahydrofurfuryl alcohol polyethylene glycol ether; polyethylene glycol; propylene glycol; 2-(2-ethoxyethoxy)ethanol; diethylene glycol monomethyl ether; alkylaryl ethers of polyethylene oxide; polyethylene oxide monomethyl ethers; polyethylene oxide dimethyl ethers; dimethyl sulfoxide; glycerol; ethyl acetate; acetoacetic ester; N-alkylpyrrolidone; terpenes; macrocyclic enhancers such as macrocyclic ketones, for example, 3-methylcyclopentadecanone, 9-cycloheptadecen-1-one, cyclohexadecanone, and cyclopentadecanone; macrocyclic esters such as pentadecalactone. Examples of suitable agents to enhance dermal penetration are disclosed at Int J Pharm. 2013 Apr. 15; 447(1-2):12-21 (incorporated herein by reference).

In particular embodiments, the topical formulation includes water. In particular embodiments, the topical formulation is substantially or totally free of water. In particular embodiments, the water content of the topical formulation is 0 to 80% water.

Other components commonly used in topical formulations include: antioxidants (e.g. α-tocopherol, β-tocopherol, γ-tocopherol, Δ-tocopherol, butylated hydroxytoluene, butylated hydroxyanisole), stabilizers, chelating agents, softeners, lubricants, emollients, pigments, fragrances, skin soothing agents, skin healing agents and skin conditioning agents. Skin healing and skin conditioning agents include glycerol, allantoin, and bisabolol.

(xiii) Pharmaceutical Formulations. In particular embodiments, lipid particles, aqueous dispersions, and/or compositions of the present disclosure can be formulated into a pharmaceutical formulation. Pharmaceutical formulations include any formulation (e.g., formulations described above) when the formulation is subject to government approval by a regulatory agency, such as the U.S. FDA.

(xiv) Common Considerations for all Products and Formulations. The following passages present considerations that are applicable to a variety of formulation types. One of ordinary skill in the art can interpret and apply these sections to formulation types described above as appropriate.

Formulations for beverages and beverage kits can include additional surfactants, glycosides, and/or water.

In particular embodiments, formulations can be diluted and then concentrated. In particular embodiments, formulations can be concentrated into a liquid or solid (e.g., spray-dried particles).

In particular embodiments, aqueous dispersions can include water soluble active compounds. In particular embodiments, water soluble active compounds include water soluble vitamins, water soluble minerals, water soluble enzymes, and substances with a negative log P. In particular embodiments, water soluble vitamins include ascorbic acid (vitamin C), thiamin, riboflavin, niacin, nicotinamide mononucleotide, pantethine, vitamin B6 (pyridoxine, pyridoxal, and pyridoxamine), folacin, folate, vitamin B12, biotin, choline, and pantothenic acid. In particular embodiments, water soluble minerals include calcium, phosphorus, potassium, sodium, chloride, magnesium, iron, zinc, iodine, sulfur, cobalt, copper, fluoride, manganese, and selenium. In particular embodiments, water soluble enzymes include bromelain, papain, lactase, beta-amylase, cellulase, lipase, and proteases. In particular embodiments, substances with a negative log P include caffeine, theanine, theobromine, theophylline, chlorogenic acids, taurine, glucuronolactone, glucose, gamma amino butyric acid (GABA), fructose, glucose, sucrose, ribose, xylitol, inositol, aspartame, rebaudoside, soluble fiber, whey protein, ethanol, carnitine, carnosine, creatine, glycerylphosphorylcholine, beta-glucan, polyphenols, amino acids, 5-HTP, glucosamine, chondroitin, collagen, hyaluronic acid, pyrroloquinoline quinone (PQQ), glutathione, fructooligosaccharides, galactooligosaccharides, and probiotic organisms.

Suitable preservatives include: benzalkonium chloride, thimerosal, chlorobutanol, methyl paraben, propyl paraben, phenylethyl alcohol, edetate disodium, sorbic acid, Onamer M, or other agents known to those skilled in the art. In particular embodiments, a suitable preservative includes any food-grade preservative. Food-grade preservatives include antioxidant preservatives and antimicrobial preservatives. Antioxidant preservatives include ascorbyl palmitate, ascorbic acid, butylated hydroxyanisole (BHA) butylated hydroxytoluene (BHT), citric acid, sulfites, tertiary butylhydroquinone (TBHQ), tocopherols, and salts and mixtures thereof. Antimicrobial preservatives include acetic acid, benzoic acid, potassium sorbate, natamycin, nisin, nitrates, nitrites, propionic acid, sorbic acid, sulfites, sodium hexametaphosphate, sulfur dioxide, and salts and mixtures thereof. In particular embodiments, preservatives include antibiotics, antimicrobials, antifungals, and antivirals. In particular embodiments, the preservative is a water-phase preservative. Water-phase preservatives include etheylenediaminetetraacetic acid (EDTA), citric acid, potassium sorbate, and sodium benzoate. In particular embodiments, preservatives may be employed at a level from 0.004% to 0.02%, from 0.001% to 0.01%, from 0.001% to 0.008%, or 0.005% to 0.006%, from 0.02% to 0.3%, from 0.04% to 0.2%, or 0.1% to 0.2% by weight of a formulation.

Suitable pH modifiers include citric acid, tartaric acid, phosphoric acid, hydrochloric acid, maleic acid, sodium hydroxide, disodium hydrogen phosphate, sodium acetate, sodium bicarbonate, sodium phosphate tribasic, dipotassium hydrogen phosphate, acetic acid, lactic acid, fumaric acid, adipic acid, sulfuric acid, salts thereof, and combinations thereof. In particular embodiments, the pH of the formulation or aqueous dispersion will be controlled. In particular embodiments, the aqueous dispersion or formulation has a pH from 2 to 9. In particular embodiments, the aqueous dispersion or formulation has a pH from 6.5 to 7.5. In particular embodiments, the aqueous dispersion or formulation has a pH of 7. Acidity can be adjusted to and maintained within the requisite range by known and conventional methods, e.g, the use of acid buffers. Typically, acidity is a balance of maximum acidity for microbial inhibition and optimum acidity for aqueous dispersion characteristics (e.g., flavor or activity). In particular embodiments, the pH modifier includes buffer to maintain a physiologically compatible pH range. Buffers include acetate, bicarbonate, citrate, and phosphate.

Coloring agents include red, black and yellow iron oxides and FD&C dyes such as FD&C Blue No. 2, FD&C Red No. 40. In particular embodiments coloring agents used in food include carotene, curcumin, riboflavin, carmine, turmeric, annatto, cochineal, betanin, saffron, paprika, elderberry, brilliant blue, indigotine, fast green, allura red, erythrosine, tartrazine, sunset yellow, and caramel.

Viscosity modifiers include cellulose or cellulose derivatives such as ethylcellulose, hydroxyethyl cellulose (HEC), hydroxypropyl cellulose (HPC), carboxymethylcellulose, sodium hydroxypropyl methylcellulose (HPMC), methylcellulose, methylethylcellulose, sodium carboxymethylcellulose, polyvinylpyrrolidone, polyvinylpolypyrrolidone (crospovidone), Carbopol (carbomer), and sodium hyaluronate (hyaluronic acid), Aerosil, cetostearyl alcohol, Gelucires 33/01, 39/01 and 43/01, glyceryl behenate, glyceryl palmitostearate, Softisans 100, 142, 378 and 649, stearyl alcohol carbomer, xanthan gum, maltodextrin, acacia, tragacanth, povidone and polyvinyl alcohol.

Lubricants include calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof.

Thickeners include xanthan gum, carboxymethylcellulose, propylene glycol alginate, gellan gum, guar gum, pectin, tragacanth gum, gum acacia, locust bean gum, as well as mixtures of these thickeners.

In particular embodiments, formulations may include a sterile unit dose type containing no preservatives, in multidose form, and/or packaged with preservatives to prevent microbial contamination during use.

Formulations can contain an amount of first LC selected from 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, or more than 100 mg.

In particular embodiments, formulations can contain a concentration of first LC ranging from 0.0001 to 10.0 w/v %, 0.005 to 10.0 w/v %, 0.01 to 10.0 w/v %, 0.05 to 10.0 w/v %, 0.1 to 10.0 w/v %, 0.5 to 10.0 w/v %, 1.0 to 10.0 w/v %, 20 to 10.0 w/v %, 3.0 to 10.0 w/v %, 4.0 to 10.0 w/v %, or 5.0 to 10.0 w/v %. These formulations may be applied several times a day, one to six times, one to four times, one to two times, or once a day.

(xv) Administration. Formulations of the present disclosure can be administered to subjects (humans, veterinary animals (dogs, cats, reptiles, birds, etc.) livestock (horses, cattle, goats, pigs, chickens, etc.) and research animals (monkeys, rats, mice, fish, etc.). In particular embodiments, an effective amount is administered to the subject.

An “effective amount” is the amount of a formulation necessary to result in a desired physiological change in the subject.

For administration, effective amounts (also referred to herein as doses) can be initially estimated based on results from in vitro assays and/or animal model studies. Such information can be used to more accurately determine useful doses in subjects of interest. The actual dose amount administered to a particular subject can be determined by the subject, a physician, veterinarian or researcher taking into account parameters such as physical and physiological factors including target, body weight, severity of a condition, gender, age, symptoms to be treated, previous or concurrent therapeutic interventions, desirable effects, route of administration, and period of administration.

The formulations of the present disclosure may be administered several times per day, one to four times per day, one to three times per day, one to two times per day, or once per day. Each administration can include one dose or several doses. The formulation can be administered as several units per administration, one to four units, one to three units, one to two units, or one units per administration. Units can be any of the administrable formulations described above including: dry powder, foams, gels, granules, inhalers, liquids, lotions, lozenges, mists, oils, ointments, pastes, pills, powder, pressurized atomizers, solutions, sprays, strips, suspensions, syrups, tablets, troches, or wafers. If the formulation is a drop, the formulation can be administered as several drops per administration, one to four drops, one to three drops, one to two drops, or one drop per administration. If the formulation is a spray, the formulation can be administered as several sprays per administration, one to four sprays, one to three sprays, one to two sprays, or one spray per administration. If the formulation is a capsule, the formulation can be administered as several capsules per administration, one to four capsules, one to three capsules, one to two capsules, or one capsule per administration. If the formulation is a tablet, the formulation can be administered as several tablets per administration, one to four tablets, one to three tablets, one to two tablets, or one tablet per administration.

Effective amounts of lipid particle-based formulations can include 104 to 109 particles/kg body weight, or 10¹ to 10¹¹ particles/kg body weight. Effective amounts to administer can include greater than 10¹ particles, greater than 10² particles, greater than 103 particles, greater than 104 particles, greater than 105 particles, greater than 10⁶ particles, greater than 107 particles, greater than 10⁸ particles, greater than 109 particles, greater than 10¹⁰ particles, or greater than 10¹¹ particles.

Effective amounts of active compounds and/or active ingredients can include 0.1 μg/kg to 5 mg/kg body weight, 0.5 μg/kg to 2 mg/kg, or 1 mg/kg to 4 mg/kg. Effective amounts to administer can include greater than 0.1 μg/kg, greater than 0.6 μg/kg, greater than 1 mg/kg, greater than 2 mg/kg, greater than 3 mg/kg, greater than 4 mg/kg, or greater than 5 mg/kg of body weight.

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (xvi) Exemplary Embodiments

1. A lipid particle including (i) at least two lipophilic components (LCs) and (ii) a surfactant, wherein at least a first LC is solid at room temperature, and at least a percentage of the surfactant is on a surface of the lipid particle.

2. The lipid particle of embodiment 1, further including a glycoside, wherein the glycoside is not a saponin if the surfactant is a saponin.

3. The lipid particle of embodiment 2, wherein the glycoside includes a terpene glycoside or saponin.

4. The lipid particle of embodiments 2 or 3, wherein the glycoside includes a rebaudioside glycoside.

5. The lipid particle of embodiment 4, wherein the rebaudioside glycoside includes rebaudioside A.

6. The lipid particle of any of embodiments 2-5, wherein the glycoside includes a mogroside-type glycoside.

7. The lipid particle of embodiment 6, wherein the mogroside-type glycoside includes mogroside V.

8. The lipid particle of any of embodiments 1-7, including at least two glycosides.

9. The lipid particle of any of embodiments 1-8, wherein the at least two LCs are from a same plant type.

10. The lipid particle of any of embodiments 1-9, wherein at least one of the at least two LCs and the surfactant are from a same plant type.

11. The lipid particle of any of embodiments 1-10, wherein a second LC is liquid at room temperature.

12. The lipid particle of any of embodiments 1-11, wherein the lipid particle is a lipid nanoparticle and/or a solid lipid particle (SLP).

13. The lipid particle of embodiments 9-12, wherein the plant type is a Cannabaceae plant.

14. The lipid particle of embodiment 13, wherein the Cannabaceae plant is a cannabis plant.

15. The lipid particle of embodiment 14, wherein the cannabis plant is Cannabis sativa, Cannabis indica, or Cannabis ruderalis.

16. The lipid particle of any of embodiments 1-15, wherein the first LC includes a cannabinoid.

17. The lipid particle of embodiment 16, wherein the cannabinoid includes tetrahydrocannabinol (THC), cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBT), tetrahydrocannabinolic acid (THCA), tetrahydrocannabivarinic acid (THCVA), and/or mixtures thereof.

18. The lipid particle of embodiments 16 or 17, wherein the cannabinoid includes CBD.

19. The lipid particle of any of embodiments 16-18, wherein the cannabinoid includes CBD and THC.

20. The lipid particle of any of embodiments 16-19, wherein the cannabinoid includes CBD and THC at a ratio of 1:1 (% w/w).

21. The lipid particle of any of embodiments 1-20, wherein at least one of the at least two LCs includes a synthetic and/or artificial cannabinoid.

22. The lipid particle of any of embodiments 11-21, wherein the second LC includes a terpene and/or a tocopherol (e.g., α-tocopherol, β-tocopherol, γ-tocopherol, and/or Δ-tocopherol).

23. The lipid particle of embodiment 22, wherein the terpene includes limonene, bisabolol, myrcene, linalool, caryophyllene, α-pinene, ρ-pinene, eucalyptol, trans-nerolido, humulene, delta 3 carene, camphene, borneol, terpineol, valencene, squalene, or geraniol.

24. The lipid particle of embodiments 22 or 23, wherein the terpene includes limonene.

25. The lipid particle of any of embodiments 22-24, wherein the terpene includes bisabolol.

26. The lipid particle of any of embodiments 22-25, wherein the terpene includes limonene and bisabolol.

27. The lipid particle of embodiment 13, wherein the Cannabaceae plant is a Humulus plant.

28. The lipid particle of embodiment 27, wherein the Humulus plant is Humulus lupulus, Humulus americanus, Humulus cordifolius, Humulus neomexicanus, Humulus pubescens, or Humulus scandens.

29. The lipid particle of embodiments 27 or 28, wherein the Humulus plant is Humulus lupulus.

30. The lipid particle of any of embodiments 1-29, wherein the first LC includes α-acids (humulone, cohumulone, adhumulone, posthumulone, prehumulone, adprehumulone), β-acids (lupulone, colupuloe, adlupulone, prelupulone, postlupulone), or flavonoids (xanthohumol, xanthan humulone).

31. The lipid particle of any of embodiments 11-30, wherein the second LC includes an essential oil, a terpene, or an alkaloid.

32. The lipid particle of embodiment 31, wherein the essential oil includes α-pinene, β-pinene, myrcene, limonene, α-humulene, β-farnesene, β-caryophyllene, α-selinene, β-selinene, or γ-muurolene.

33. The lipid particle of embodiment 31, wherein the terpene includes monoterpene (C10) myrcene, sesquiterpenes (C15) caryophyllene, or humulene.

34. The lipid particle of embodiment 31, wherein the alkaloid includes a lupuline.

35. The lipid particle of any of embodiments 9-12, 16-26, or 30-34, wherein the plant type is a Rubiaceae plant.

36. The lipid particle of embodiment 35, wherein the Rubiaceae plant is a Coffea plant.

37. The lipid particle of embodiment 36, wherein the Coffea plant is Coffea abbayesii, Coffea affinis, Coffea alleizettii, Coffea ambanjensis, Coffea ambongenis, Coffea andrambovatensis, Coffea ankaranensis, Coffea anthonyi, Coffea arabica, Coffea arenesiana, Coffea augagneurii, Coffea bakossii, Coffea benghalensis, Coffea bertrandii, Coffea betamponensis, Coffea bissetiae, Coffea boinensis, Coffea boiviniana, Coffea bonnieri, Coffea brassii, Coffea brevipes, Coffea bridsoniae, Coffea buxifolia, Coffea canephora, Coffea carrissoi, Coffea charrieriana, Coffea cochinchinensis, Coffea commersoniana, Coffea congensis, Coffea costatifructa, Coffea coursiana, Coffea dactylifera, Coffea decaryana, Coffea dubardii, Coffea ebracteolata, Coffea eugenioides, Coffea fadenii, Coffea farafanganensis, Coffea floresiana, Coffea fotsoana, Coffea fragilis, Coffea fragrans, Coffea gallienii, Coffea grevei, Coffea heimii, Coffea homollei, Coffea horsfieldiana, Coffea humbertii, Coffea humblotiana, Coffea humilis, Coffea jumellei, Coffea kapakata, Coffea kianjavatensis, Coffea kihansiensis, Coffea kimbozensis, Coffea kivuensis, Coffea labatii, Coffea lancifolia, Coffea lebruniana, Coffea leonimontana, Coffea Ieroyi, Coffea liaudii, Coffea liberica, Coffea ligustroides, Coffea littoralis, Coffea lulandoensis, Coffea mabesae, Coffea macrocarpa, Coffea madurensis, Coffea magnistipula, Coffea malabarica, Coffea mangoroensis, Coffea mannii, Coffea manombensis, Coffea mapiana, Coffea mauritiana, Coffea mayombensis, Coffea mcphersonii, Coffea melanocarpa, Coffea merguensis, Coffea millotii, Coffea minutiflora, Coffea mogenetii, Coffea mongensis, Coffea montekupensis, Coffea montis-sacri, Coffea moratii, Coffea mufindiensis, Coffea myrtifolia, Coffea namorokensis, Coffea neobridsoniae, Coffea neoleroyi, Coffea perrieri, Coffea pervilleana, Coffea pocsii, Coffea pseudozanguebariae, Coffea pterocarpan, Coffea racemose, Coffea rakotonasoloi, Coffea ratsimamangae, Coffea resinosa, Coffea rhamnifolia, Coffea richardii, Coffea sahafaryensis, Coffea sakarahae, Coffea salvatrix, Coffea sambavensis, Coffea sapinii, Coffea schliebenii, Coffea semsei, Coffea sessiliflora, Coffea stenophylla, Coffea tetragona, Coffea togoensis, Coffea toshii, Coffea travancorensis, Coffea tricalysioides, Coffea tsirananae, Coffea vatovavyensis, Coffea vavateninensis, Coffea vianneyi, Coffea vohemarensis, Coffea wightiana, or Coffea zanguebariae.

38. The lipid particle of embodiments 36 or 37, wherein the Coffea plant is Coffea arabica.

39. The lipid particle of any of embodiments 1-38, wherein the first LC includes tryptophan alkaloids or diterpenes.

40. The lipid particle of any of embodiments 11-39, wherein the second LC includes a linoleic acid, a palmitic acid, a triacylglycerol, a terpene ester, or a terpene.

41. The lipid particle of any of embodiments 9-12, 16-26, 30-34, or 39-40, wherein the plant type is a Theaceae plant.

42. The lipid particle of embodiment 41, wherein the Theaceae plant is a Camellia plant.

43. The lipid particle of embodiment 42, wherein the Camellia plant is Camellia sinensis var. sinensis, Camellia sinensis var. assamica, Camellia sinensis var. pubilimba, or Camellia sinensis var. dehungensis.

44. The lipid particle of embodiments 42 or 43, wherein the Camellia plant is Camellia sinensis var. sinensi.

45. The lipid particle of any of embodiments 1-44 wherein the first LC includes resveratrol or proanthocyanidins.

46. The lipid particle of any of embodiments 11-45, wherein the second LC includes a terpene, a terpenoid ester, a diacylglycerol, a sterol ester, or a β-amyrin ester.

47. The lipid particle of embodiment 46, wherein the terpene includes a squalene.

48. The lipid particle of any of embodiments 1-47 wherein the first LC includes coenzyme Q10, plastoquinone, or plastoquinonol.

49. The lipid particle of embodiment 48, wherein coenzyme Q10 includes ubiquinone or ubiquinol.

50. The lipid particle of any of embodiments 9-34, 39-40, or 45-49, wherein the plant type is a Cannabaceae plant and the surfactant includes a hemp protein.

51. The lipid particle of any of embodiments 1-15, 21-50 wherein the first LC includes a lipophilic protein or peptide.

52. The lipid particle of any of embodiment 1-15, 21-51, wherein the first LC includes a lipophilic enzyme.

53. The lipid particle of embodiment 52, wherein the lipophilic enzyme includes nattokinase or bromelain.

54. The lipid particle of any of embodiments 1-53, wherein the lipid particle further includes a preservative.

55. The lipid particle of embodiment 54, wherein at least one of the at least two LCs or the preservative is solid at room temperature; and at least a percentage of the surfactant and preservative is on the surface of the lipid particle.

56. The lipid particle of any of embodiments 1-55, wherein the lipid particle further includes a stabilizer.

57. The lipid particle of embodiment 56, wherein the stabilizer is solid at room temperature.

58. The lipid particle of embodiment 56 or 57, wherein at least a percentage of the stabilizer is on the surface of the lipid particle.

59. The lipid particle of any of embodiments 1-58, wherein at least one of the at least two LCs includes both (i) an active compound and (ii) a carrier or viscosity modifier.

60. The lipid particle of any of embodiments 12-59, wherein the SLP further includes a third LC from a same plant type, wherein the third LC is solid or liquid at room temperature.

61. The lipid particle of embodiment 60, wherein the third LC is present within the SLPs at a same % w/w as the second LC.

62. The lipid particle of any of embodiments 1-61, wherein the surfactant includes a glycoside.

63. The lipid particle of any of embodiments 1-61, wherein the surfactant includes a sorbitan fatty acid ester, polyethylene glycol sorbitan fatty acid ester, polyethylene-polypropylene glycol, saturated polyglycolized glyceride, polyethylene glycol, quillaia, polyethylene glycol stearate, polyethylene glycol hydrogenated castor oil, diethylene glycol monoethyl ether, propylene glycol laurate, D-α-Tocopherol polyethylene glycol 1000 succinate (TPGS), polyvinyl pyrrolidone, iota carrageenan, hydroxypropyl methylcellulose (HPMC), carboxymethyl cellulose (CMC), microcrystalline cellulose (MCC), lecithin, sucrose laurate, sucrose palmitate, sucrose stearate, gamma-cyclodextrin, β-cyclodextrin pectin, whey protein, caseinate, Xanthan gum, locust Bean gum, gellan gum, acacia gum, guar gum, Tragacanth gum, polyethylene glycol hydroxystearate polyoxyl-10-oleyl ether, or polyethylene glycol glyceride.

64. The lipid particle of any of embodiments 1-61 or 63, wherein the surfactant includes polysorbate 80.

65. The lipid particle of any of embodiments 1-61 or 63-64, wherein the surfactant includes sorbitan monooleate.

66. The lipid particle of any of embodiments 1-61 or 63-65, wherein the surfactant includes polysorbate 80 and sorbitan monooleate.

67. The lipid particle of any of embodiments 12-66, wherein a hydrophobic tail of the surfactant is embedded within the surface of the SLP.

68. The lipid particle of any of embodiments 1-67, within a formulation formulated for oral administration.

69. The lipid particle of embodiment 68, wherein the formulation formulated for oral administration is flavored.

70. The lipid particle of any of embodiments 1-67, within a formulation formulated for mucosal administration.

71. The lipid particle of any of embodiments 1-67, within a formulation formulated for ocular administration.

72. The lipid particle of any of embodiments 1-67, within a formulation formulated for topical administration.

73. The lipid particle of any of embodiments 1-67, within a pharmaceutical formulation.

74. The lipid particle of embodiment 73, wherein the pharmaceutical formulation is flavored.

75. The lipid particle of embodiment 73 or 74, wherein the pharmaceutical formulation includes a tablet, capsule or sachet.

76. The lipid particle of any of embodiments 1-67, within a food product.

77. The lipid particle of any of embodiments 1-67, within a formulation formulated for a consumer product.

78. The lipid particle of any of embodiments 1-67, within an aqueous dispersion.

79. The lipid particle of any of embodiments 1-67, within a dry composition.

80. The lipid particle of any of embodiments 1-79, wherein a ratio of first LC and second LC to surfactant is at least 2:1, at least 3:1, or at least 9:1 (% w/w).

81. The lipid particle of any of embodiments 1-80, wherein a ratio of first LC to second LC to surfactant is 10:8:5, 10:4:5, or 5:4:4 (% w/w).

82. The lipid particle of any of embodiments 1-81, including two, three, or four surfactants.

83. The lipid particle of any of embodiments 1-82, wherein a ratio of first LC and second LC to surfactant is greater than 18:1, 9:1, 18:3, 72:11, 9:2, 18:5, 36:11, 6:2, 18:7, 9:4, 6:3, or 9:5 (% w/w).

84. The lipid particle of any of embodiments 1-83, wherein a ratio of first LC to surfactant is 10:1, 20:3, 5:1, 20:5, 40:11 10:3, 20:7, 5:2, 9:4, 20:9, 2:1, 20:11, 5:3, 20:13, 10:7, 20:15, 5:4, 20:17, 5:4, 10:9, or 1:1 (% w/w).

85. The lipid particle of any of embodiments 1-84, wherein a ratio of first LC to second LC to the surfactant 5:4:2, 20:16:11, 10:8:7, 5:4:4, 20:12:11, 5:2:4, 40:32:11, or 10:4:7 (% w/w).

86. The lipid particle of any of embodiments 2-85, wherein a ratio of first LC and second LC to glycoside is at least 1:1, 1.5:1, 2:1, 5:1, 6:1, 7:1, 8:1, 9:1, or 10:1 (% w/w).

87. The lipid particle of any of embodiments 78-86, wherein polysorbate 80 is 3% w/w or less of the aqueous dispersion.

88. The lipid particle of any of embodiments 78-87, wherein polysorbate 80 is 2% w/w or less of the aqueous dispersion.

89. The lipid particle of any of embodiments 78-88, wherein polysorbate 80 is 1% w/w or less of the aqueous dispersion.

90. The lipid particle of any of embodiments 78-89, wherein sorbitan monooleate is 6% w/w or less of the aqueous dispersion.

91. The lipid particle of any of embodiments 78-90, wherein sorbitan monooleate is 4% w/w or less of the aqueous dispersion.

92. The lipid particle of any of embodiments 78-91, wherein sorbitan monooleate is 2% w/w or less of the aqueous dispersion.

93. The lipid particle of any of embodiments 78-88, or 90-91, wherein sorbitan monooleate is 3.6% w/w or less of the aqueous dispersion and polysorbate 80 is 2% w/w or less of the aqueous dispersion.

94. The lipid particle of any of embodiments 78-87, or 90, wherein sorbitan monooleate is 5.2% or less of the aqueous dispersion and polysorbate 80 is 3% w/w or less of the aqueous dispersion.

95. The lipid particle of any of embodiments 78-94, wherein sorbitan monooleate is 1.8% or less of the aqueous dispersion and polysorbate 80 is 1% w/w or less of the aqueous dispersion.

96. The lipid particle of any of embodiments 1-95, wherein the surfactant is 31% w/w or less of the lipid particle.

97. The lipid particle of any of embodiments 1-96, wherein polysorbate 80 is 12% w/w or less of the lipid particle.

98. The lipid particle of any of embodiments 1-97, wherein polysorbate 80 is 9% w/w or less of the lipid particle.

99. The lipid particle of any of embodiments 1-98, wherein polysorbate 80 is 5% w/w or less of the lipid particle.

100. The lipid particle of any of embodiments 1-99, wherein sorbitan monooleate is 20% w/w or less of the lipid particle.

101. The lipid particle of any of embodiments 1-100, wherein sorbitan monooleate is 15% w/w or less of the lipid particle.

102. The lipid particle of any of embodiments 1-101, wherein sorbitan monooleate is 9% w/w or less of the lipid particle.

103. The lipid particle of any of embodiments 1-98, or 100-101, wherein sorbitan monooleate is 15.0% w/w or less of the lipid particle and polysorbate 80 is 8.5% w/w or less of the lipid particle.

104. The lipid particle of any of embodiments 1-97 or 100, wherein sorbitan monooleate is 19.7% or less of the lipid particle and polysorbate 80 is 11.1% w/w or less of the lipid particle.

105. The lipid particle of any of embodiments 1-104, wherein sorbitan monooleate is 8.5% or less of the lipid particle and polysorbate 80 is 4.8% w/w or less of the lipid particle.

106. The lipid particle of any of embodiments 2-105, wherein the glycoside is 40% or less of the lipid particle.

107. The lipid particle of any of embodiments 2-106, wherein the glycoside is 30% or less of the lipid particle.

108. The lipid particle of any of embodiments 2-107, wherein the glycoside is 20% or less of the lipid particle.

109. The lipid particle of embodiment 78, wherein the aqueous dispersion includes water.

110. The lipid particle of embodiment 109, wherein the water includes deionized water, alkaline water, purified water, electrolyte water, carbonated water, isotonic water, or mineral water.

111. The lipid particle of any of embodiments 78-110, wherein the aqueous dispersion includes a ratio of first LC and second LC to surfactant of at least 18:1, 9:1, 18:3, 72:11, 9:2, 18:5, 36:11, 6:2, 18:8, 18:7, 9:4, 2:1, 9:5, or 1:1 (% w/w).

112. The lipid particle of any of embodiments 1-111, wherein a ratio of first LC and second LC to surfactant is 2:1 (% w/w).

113. The lipid particle of any of embodiments 1-111, wherein a ratio of first LC and second LC to surfactant is 18:8 (% w/w).

114. The lipid particle of any of embodiments 1-111, wherein a ratio of first LC and second LC to surfactant is 36:11 (% w/w).

115. The lipid particle of any of embodiments 1-111, wherein a ratio of first LC and second LC to surfactant is 72:11.

116. The lipid particle of any of embodiments 78-115, wherein the aqueous dispersion includes a ratio of first LC to surfactant of at least 10:1, 20:3, 5:1, 20:5, 40:11 10:3, 20:7, 5:2, 9:4, 20:9, 2:1, 20:11, 5:3, 20:13, 10:7, 20:15, 5:4, 20:17, 10:9, or 1:1 (% w/w).

117. The lipid particle of any of embodiments 1-116, wherein a ratio of first LC to surfactant is 5:4 (% w/w).

118. The lipid particle of any of embodiments 1-116, wherein a ratio of first LC to surfactant is 20:11 (% w/w).

119. The lipid particle of any of embodiments 1-116, wherein a ratio of first LC to surfactant is 40:11 (% w/w).

120. The lipid particle of any of embodiments 78-119, wherein the aqueous dispersion includes a ratio of first LC to second LC to surfactant of at least 5:4:2, 20:16:11, 10:8:7, 5:4:4, 20:12:11, 5:2:4, 40:32:11, or 10:4:7 (% w/w).

121. The lipid particle of any of embodiments 1-120, wherein a ratio of first LC to second LC to surfactant is 5:4:4 (% w/w).

122. The lipid particle of any of embodiments 1-80, or 82-120, wherein a ratio of first LC to second LC to surfactant is 20:16:11 (% w/w).

123. The lipid particle of any of embodiments 1-80, or 82-120, wherein a ratio of first LC to second LC to surfactant is 40:32:11 (% w/w).

124. The lipid particle of any of embodiments 78-123, wherein the aqueous dispersion includes a ratio of first LC and second LC to glycoside of at least 1:1, 1.5:1, 2:1, 5:1, 6:1, 7:1, 8:1, 9:1, 10:1 (w/w).

125. The lipid particle of any of embodiments 1-124, wherein the room temperature is 20° C. to 25° C.

126. The lipid particle of embodiment 1, wherein the lipid particle includes 1-20% CoQ10, 0.1-2% Rebaudioside A, 0.1-2% Mogroside V, 0.1-3% Saponin, 0.005-0.5% Sodium Benzoate, and 0.005-0.5% Potassium Sorbate.

127. The lipid particle of embodiment 1, wherein the lipid particle includes 1-20% CBD, 1-20% α-Tocopherol, 0.01-1% Ascorbyl Palmitate, 1-10% Span 80, 0.1-2% Mogroside V, 0.1-2% Rebaudioside A, 0.1-10% Tween 80, 0.005-0.5% Sodium Benzoate, 0.005-0.5% Potassium Sorbate.

128. The lipid particle of embodiment 1, wherein the lipid particle includes 5-15% CoQ10, 0.2-1% Rebaudioside A, 0.2-1% Mogroside V, 0.5-2% Saponin, 0.01-0.1% Sodium Benzoate, and 0.01-0.1% Potassium Sorbate.

129. The lipid particle of embodiment 1, wherein the lipid particle includes 5-15% CBD, 4-12% α-Tocopherol, 0.05-0.5% Ascorbyl Palmitate, 2-6% Span 80, 0.2-1% Mogroside V, 0.2-1% Rebaudioside A, 0.5-5% Tween 80, 0.01-0.1% Sodium Benzoate, 0.01-0.1% Potassium Sorbate.

130. The lipid particle of embodiment 1, wherein the lipid particle includes 10% CoQ10, 0.61% Rebaudioside A, 0.82% Mogroside V, 1% Saponin, 0.03% Sodium Benzoate, and 0.04% Potassium Sorbate.

131. The lipid particle of embodiment 1, wherein the lipid particle includes 10% CBD, 8% α-Tocopherol, 0.1% Ascorbyl Palmitate, 3.52% Span 80, 0.82% Mogroside V, 0.62% Rebaudioside A, 1.98% Tween 80, 0.03% Sodium Benzoate, 0.04% Potassium Sorbate.

132. A lipid particle including (i) a first lipophilic component (LC) that is solid at room temperature and (ii) a surfactant, wherein a ratio of LC to surfactant is at least 1:1 (% w/w).

133. A lipid particle including (i) a first lipophilic component (LC) that is solid at room temperature, (ii) no additional lipid carrier and (iii) a surfactant, wherein a ratio of LC to surfactant is at least 1:1 (% w/w).

134. A lipid particle of embodiment 132 or 122, wherein the surfactant includes a glycoside.

135. A lipid particle including (i) a first lipophilic component (LC) that is solid at room temperature, (ii) a surfactant, and (iii) a glycoside, wherein a ratio of LC to surfactant is at least 1:1 (% w/w) and wherein the glycoside is not a saponin if the surfactant is a saponin.

136. A lipid particle including (i) a first lipophilic component (LC) that is solid at room temperature, (ii) no additional lipid carrier, (iii) a surfactant, and (iv) a glycoside, wherein a ratio of LC to surfactant is at least 1:1 (% w/w) and wherein the glycoside is not a saponin if the surfactant is a saponin.

137. The lipid particle of any of embodiments 1-136, wherein the LC includes a lipid active compound (LAC).

138. The lipid particle of embodiment 137, wherein the LAC includes a regulated LAC (rLAC)

139. A plurality of lipid particles of any of embodiments 1-138, including at least two types of lipid particles, wherein the types of lipid particles have LCs from different plant types and a common surfactant type.

140. The plurality of lipid particles of embodiment 139, wherein a ratio of a first lipid particle type to a second lipid particle type is 10:1, 9:1, 8:1, 7:1, 6:1, 5:1, 4:1, 3:1, 2:1, or 1:1.

141. The plurality of lipid particles of embodiments 139 or 140, including at least three types of lipid particles, wherein the three types of lipid particles include a first LC in the first lipid particle type, a second LC in the second lipid particle type, and a third LC in a third lipid particle type and a common surfactant type.

142. The plurality of lipid particles of embodiment 141, wherein a ratio of the first lipid particle type to second lipid particle type to third lipid particle type is 10:1:1, 9:1:1, 8:1:1, 7:1:1, 6:1:1, 5:1:1, 4:1:1, 3:1:1, 2:1:1, 1:1:1, 10:2:1, 9:1:5, 8:4:1, 7:1:4, 6:6:1, 5:3:1, 4:1:3, 3:1:3, 2:5:1, or 1:8:1.

143. The plurality of lipid particles of any of embodiments 139-142, wherein the common surfactant type is an anionic surfactant type.

144. The plurality of lipid particles of any of embodiments 139-142, wherein the common surfactant type is a cationic surfactant type.

145. The plurality of lipid particles of any of embodiments 139-142, wherein the common surfactant type is a non-ionic surfactant type.

146. The plurality of lipid particles of any of embodiments 139-142, wherein the common surfactant type is a zwitterionic surfactant type.

147. The plurality of lipid particles of any of embodiments 139-146, wherein the different plant types are selected from a Cannabaceae plant, a Rubiaceae plant, and a Theaceae plant.

148. The plurality of lipid particles of any of embodiments 139-147, wherein the different plant types are selected from different cannabis plant species.

149. The plurality of lipid particles of embodiment 148, wherein the different cannabis plant species are selected from Cannabis sativa, Cannabis indica, and Cannabis ruderalis.

150. A beverage kit including a lipid particle of any of embodiments 1-138 or a plurality of lipid particles of any of embodiments 139-149 and a beverage.

151. The beverage kit of embodiment 150, wherein the beverage includes a surfactant.

152. The beverage kit of embodiment 150, wherein the surfactant within the beverage is at an amount that creates a dispersion of the lipid particles within the beverage following an addition of the lipid particles to the beverage.

153. The beverage kit of any of embodiments 150-152, wherein the beverage is beer, coffee, or tea.

154. The beverage kit of any of embodiments 150-153, wherein the beverage is soda.

155. The beverage kit of any of embodiments 150-154, wherein the beverage is a juice-flavored drink.

156. The beverage kit of any of embodiments 150-155, wherein the beverage is carbonated water.

157. A formulation including lipid particles wherein the lipid particles include (i) an LC and (ii) a surfactant, and wherein a ratio of the LC to the surfactant within the formulation is at least 2:1 (% w/w).

158. The formulation of embodiment 157, wherein the surfactant includes a glycoside.

159. The formulation of embodiments 157 or 158, wherein the lipid particles are lipid nanoparticles and/or solid lipid particles (SLPs).

160. The formulation of any of embodiments 157-159, wherein the formulation includes an aqueous dispersion, an aqueous composition, a liquid composition, or a dry composition.

161. The formulation of any of embodiments 157-160, wherein the formulation further includes a glycoside, wherein the glycoside is not a saponin if the surfactant is a saponin.

162. The formulation of embodiment 161, wherein the lipid particle includes the glycoside.

163. The formulation of embodiment 161 or 162, wherein the glycoside includes at least two glycosides.

164. The formulation of any of embodiments 161-163, wherein the glycoside includes a terpene glycoside or saponin.

165. The formulation of any of embodiments 161-164, wherein the glycoside includes a rebaudioside glycoside.

166. The formulation of embodiment 165, wherein the rebaudioside glycoside includes rebaudioside A.

167. The formulation of any of embodiments 161-166, wherein the glycoside includes a mogroside-type glycoside.

168. The formulation of embodiment 167, wherein the mogroside-type glycoside includes mogroside V.

169. The formulation of any of embodiments 157-168, wherein the LC includes a first LC from a Cannabaceae plant, a Rubiaceae plant, and/or a Theaceae plant.

170. The formulation of embodiment 169, wherein the first LC is from the Cannabaceae plant.

171. The formulation of embodiment 169 or 170, wherein the Cannabaceae plant is a cannabis plant.

172. The formulation of embodiment 171, wherein the cannabis plant is Cannabis sativa, Cannabis indica, or Cannabis ruderalis.

173. The formulation of any of embodiments 169-172, wherein the first LC includes a cannabinoid.

174. The formulation of embodiment 173, wherein the cannabinoid includes tetrahydrocannabinol (THC), cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBT), tetrahydrocannabinolic acid (THCA), tetrahydrocannabivarinic acid (THCVA), and/or mixtures thereof.

175. The formulation of embodiments 173 or 174, wherein the cannabinoid includes CBD.

176. The formulation of any of embodiments 173-175, wherein the cannabinoid includes CBD and THC.

177. The formulation of any of embodiments 173-176, wherein the cannabinoid includes CBD and THC at a ratio of 1:1 (% w/w).

178. The formulation of any of embodiments 157-177, wherein the LC includes a synthetic and/or artificial cannabinoid.

179. The formulation embodiment 178, wherein the synthetic and/or artificial cannabinoid includes nabilone; 7-OH-CBD; 7-OH-CBDV; 3-carbamoyl-2-pyridone; carenadiol; cannabinoid carboxylic acids; pyrido[3,2-E][1,2,4]triazolo[4,3-C]pyrimidine; tetrahydro-pyrazolo[3,4-C] pyridine; bicyclo[3.1.1]heptan-2-one cannabinoid; resorcinol; dexanabinol compounds; cannabimimetic lipid amide compounds; 2-oxoquinolone compounds; 3,4-diaryl-4,5-dihydro-(h)-pyrazole-1-carboxamide; and a derivative and/or analog thereof.

180. The formulation of embodiments 169 or 170, wherein the Cannabaceae plant is a Humulus plant.

181. The formulation of embodiment 180, wherein the Humulus plant is Humulus lupulus, Humulus americanus, Humulus cordifolius, Humulus neomexicanus, Humulus pubescens, or Humulus scandens.

182. The formulation of embodiments 180 or 181, wherein the Humulus plant is Humulus lupulus.

183. The formulation of any of embodiments 169-182, wherein the first LC includes α-acids (humulone, cohumulone, adhumulone, posthumulone, prehumulone, adprehumulone), β-acids (lupulone, colupuloe, adlupulone, prelupulone, postlupulone), or flavonoids (xanthohumol or xanthan humulone).

184. The formulation of embodiment 169, wherein the first LC is from the Rubiaceae plant.

185. The formulation of embodiment 184, wherein the Rubiaceae plant is a Coffea plant.

186. The formulation of embodiment 185, wherein the Coffea plant is Coffea abbayesii, Coffea affinis, Coffea alleizettii, Coffea ambanjensis, Coffea ambongenis, Coffea andrambovatensis, Coffea ankaranensis, Coffea anthonyi, Coffea arabica, Coffea arenesiana, Coffea augagneurii, Coffea bakossii, Coffea benghalensis, Coffea bertrandii, Coffea betamponensis, Coffea bissetiae, Coffea boinensis, Coffea boiviniana, Coffea bonnieri, Coffea brassii, Coffea brevipes, Coffea bridsoniae, Coffea buxifolia, Coffea canephora, Coffea carrissoi, Coffea charrieriana, Coffea cochinchinensis, Coffea commersoniana, Coffea congensis, Coffea costatifructa, Coffea coursiana, Coffea dactylifera, Coffea decaryana, Coffea dubardii, Coffea ebracteolata, Coffea eugenioides, Coffea fadenii, Coffea farafanganensis, Coffea floresiana, Coffea fotsoana, Coffea fragilis, Coffea fragrans, Coffea gallienii, Coffea grevei, Coffea heimii, Coffea homollei, Coffea horsfieldiana, Coffea humbertii, Coffea humblotiana, Coffea humilis, Coffea jumellei, Coffea kapakata, Coffea kianjavatensis, Coffea kihansiensis, Coffea kimbozensis, Coffea kivuensis, Coffea labatii, Coffea lancifolia, Coffea lebruniana, Coffea leonimontana, Coffea Ieroyi, Coffea liaudii, Coffea liberica, Coffea ligustroides, Coffea littoralis, Coffea lulandoensis, Coffea mabesae, Coffea macrocarpa, Coffea madurensis, Coffea magnistipula, Coffea malabarica, Coffea mangoroensis, Coffea mannii, Coffea manombensis, Coffea mapiana, Coffea mauritiana, Coffea mayombensis, Coffea mcphersonii, Coffea melanocarpa, Coffea merguensis, Coffea millotii, Coffea minutiflora, Coffea mogenetii, Coffea mongensis, Coffea montekupensis, Coffea montis-sacri, Coffea moratii, Coffea mufindiensis, Coffea myrtifolia, Coffea namorokensis, Coffea neobridsoniae, Coffea neoleroyi, Coffea perrieri, Coffea pervilleana, Coffea pocsii, Coffea pseudozanguebariae, Coffea pterocarpan, Coffea racemose, Coffea rakotonasoloi, Coffea ratsimamangae, Coffea resinosa, Coffea rhamnifolia, Coffea richardii, Coffea sahafaryensis, Coffea sakarahae, Coffea salvatrix, Coffea sambavensis, Coffea sapinii, Coffea schliebenii, Coffea semsei, Coffea sessiliflora, Coffea stenophylla, Coffea tetragona, Coffea togoensis, Coffea toshii, Coffea travancorensis, Coffea tricalysioides, Coffea tsirananae, Coffea vatovavyensis, Coffea vavateninensis, Coffea vianneyi, Coffea vohemarensis, Coffea wightiana, or Coffea zanguebariae.

187. The formulation of embodiments 185 or 186, wherein the Coffea plant is Coffea arabica.

188. The formulation of any of embodiments 169-187, wherein the first LC includes tryptophan alkaloids or diterpenes.

189. The formulation of embodiment 169, wherein the first LC is from the Theaceae plant.

190. The formulation of embodiment 189, wherein the Theaceae plant is a Camellia plant.

191. The formulation of embodiment 190, wherein the Camellia plant is Camellia sinensis var. sinensis, Camellia sinensis var. assamica, Camellia sinensis var. pubilimba, or Camellia sinensis var. dehungensis.

192. The formulation of embodiments 190 or 191, wherein the Camellia plant is Camellia sinensis var. sinensi.

193. The formulation of any of embodiments 169-192, wherein the first LC includes a resveratrol or proanthocyanidins.

194. The formulation of any of embodiments 169-193, wherein the first LC includes a lipophilic protein or peptide.

195. The formulation of any of embodiments 169-194, wherein the first LC includes a lipophilic enzyme.

196. The formulation of any of embodiments 169-195, wherein the first LC includes nattokinase or bromelain.

197. The formulation of any of embodiments 169-196, wherein the LC includes a second LC.

198. The formulation of embodiment 197, wherein the second LC is derived from a same plant family as the first LC.

199. The formulation of embodiments 197 or 198, wherein the second LC is derived from a same plant genus as the first LC.

200. The formulation of any of embodiments 197-199, wherein the second LC is derived from a same plant species as the first LC.

201. The formulation of any of embodiments 197-200, wherein the second LC is present at less than half % w/w of the first LC.

202. The formulation of any of embodiments 197-201, wherein the LC further includes a third LC.

203. The formulation of embodiment 202, wherein the third LC is derived from a same plant family as the first LC.

204. The formulation of embodiments 202 or 203, wherein the third LC is derived from a same plant genus as the first LC.

205. The formulation of any of embodiments 202-204, wherein the third LC is derived from a same plant species as the first LC.

206. The formulation of any of embodiments 202-205, wherein the second LC and the third LC are derived from a same plant family as the first LC.

207. The formulation of any of embodiments 202-206, wherein the second LC and the third LC are derived from a same plant genus as the first LC.

208. The formulation of any of embodiments 202-207, wherein the second LC and the third LC are derived from a same plant species as the first LC.

209. The formulation of any of embodiments 200-208, wherein the plant species is Cannabis sativa, Humulus lupulus, Coffea arabica, or Camellia sinensis.

210. The formulation of any of embodiments 200-209, wherein the plant species is Cannabis sativa.

211. The formulation of any of embodiments 202-210, wherein the third LC is present within the SLPs at a same % w/w as the second LC.

212. The formulation of any of embodiments 202-211, wherein the second LC and/or the third LC include(s) a Cannabaceae plant component.

213. The formulation of embodiment 212 wherein the Cannabaceae plant component includes a terpene, an essential oil, a tocopherol, or an alkaloid.

214. The formulation of embodiment 213, wherein the terpene includes limonene, bisabolol, myrcene, linalool, caryophyllene, α-pinene, β-pinene, eucalyptol, trans-nerolido, humulene, delta 3 carene, camphene, borneol, terpineol, valencene, squalene, monoterpene (C10) myrcene, sesquiterpenes (C15) caryophyllene, or geraniol.

215. The formulation of embodiment 213, wherein the essential oil includes α-pinene, β-pinene, myrcene, limonene, α-humulene, β-farnesene, β-caryophyllene, α-selinene, β-selinene, or γ-muurolene.

216. The formulation of embodiment 213, wherein the tocopherol includes α-tocopherol, β-tocopherol, γ-tocopherol, or Δ-tocopherol.

217. The formulation of embodiment 213, wherein the alkaloid includes a lupuline.

218. The formulation of embodiment 213 or 214, wherein the terpene includes monoterpene (C10) myrcene, sesquiterpenes (C15) caryophyllene, or humulene.

219. The formulation of any of embodiments 197-218, wherein the first LC includes a cannabinoid and the second LC includes a humulene.

220. The formulation of any of embodiments 197-219, wherein the first LC includes a cannabinoid and the second LC includes a tocopherol.

221. The formulation of any of embodiments 202-220, wherein the second LC and/or the third LC include(s) a Rubiaceae plant component.

222. The formulation of embodiment 221, wherein the Rubiaceae plant component includes a linoleic acid, a palmitic acid, a triacylglycerol, a terpene ester, or a terpene.

223. The formulation of any of embodiments 202-170, wherein the second LC and/or the third LC include(s) a Theaceae plant component.

224. The formulation of embodiment 223, wherein the Theaceae plant component includes a terpene, a terpenoid ester, a diacylglycerol, a sterol ester, or a β-amyrin ester.

225. The formulation of embodiment 224, wherein the terpene includes squalene.

226. The formulation of any of embodiments 202-225, wherein the second LC and/or the third LC include(s) olive oil, limonene, sesame oil, coconut oil, hemp oil, sunflower oil, or corn oil.

227. The formulation of any of embodiments 169-226, wherein the first LC includes coenzyme Q10, plastoquinone, or plastoquinol.

228. The formulation of embodiment 227, wherein the coenzyme Q10 includes ubiquinol or ubiquinone.

229. A formulation of any of embodiments 157-228, wherein the surfactant includes a glycoside.

230. The formulation of any of embodiments 157-228, wherein the surfactant includes a sorbitan fatty acid ester, polyethylene glycol sorbitan fatty acid ester, polyethylene-polypropylene glycol, saturated polyglycolized glyceride, polyethylene glycol, quillaia, polyethylene glycol stearate, polyethylene glycol hydrogenated castor oil, diethylene glycol monoethyl ether, propylene glycol laurate, D-α-Tocopherol polyethylene glycol 1000 succinate (TPGS), polyvinyl pyrrolidone, iota carrageenan, hydroxypropyl methylcellulose (HPMC), carboxymethyl cellulose (CMC), microcrystalline cellulose (MCC), lecithin, sucrose laurate, sucrose palmitate, sucrose stearate, gamma-cyclodextrin, β-cyclodextrin pectin, whey protein, caseinate, Xanthan gum, locust Bean gum, gellan gum, acacia gum, guar gum, Tragacanth gum, polyethylene glycol hydroxystearate polyoxyl-10-oleyl ether, or polyethylene glycol glyceride.

231. The formulation of any of embodiments 157-230, wherein the surfactant includes polysorbate 80.

232. The formulation of any of embodiments 157-231, wherein the surfactant includes sorbitan monooleate.

233. The formulation of any of embodiments 157-232, wherein the surfactant includes polysorbate 80 and sorbitan monooleate.

234. The formulation of any of embodiments 160-233, wherein the aqueous dispersion or liquid composition includes a water soluble active compound.

235. The formulation of any of embodiments 159-234, wherein a hydrophobic tail of the surfactant is embedded within a surface of the SLP.

236. The formulation of any of embodiments 157-235, formulated for oral administration.

237. The formulation of any of embodiments 157-235, formulated for mucosal administration.

238. The formulation of any of embodiments 157-235, formulated for ocular administration.

239. The formulation of any of embodiments 157-235, formulated for topical administration.

240. The formulation of embodiment 157-235, formulated as a pharmaceutical composition.

241. The formulation of any of embodiments 157-235, formulated as a food product.

242. The formulation of any of embodiments 157-241, formulated onto a consumer product.

243. The formulation of any of embodiments 157-242, including 200 mg first LC with 3 mg or less of surfactant.

244. The formulation of any of embodiments 157-243, including 100 mg first LC with 3 mg or less of surfactant.

245. The formulation of any of embodiments 157-244, including 200 mg cannabinoid, α-acids (humulone, cohumulone, adhumulone, posthumulone, prehumulone, adprehumulone), β-acids (lupulone, colupuloe, adlupulone, prelupulone, postlupulone), or flavonoids (xanthohumol or xanthan humulone) with 3 mg or less of surfactant.

246. The formulation of any of embodiments 157-245, including 100 mg cannabinoid, α-acids (humulone, cohumulone, adhumulone, posthumulone, prehumulone, adprehumulone), β-acids (lupulone, colupuloe, adlupulone, prelupulone, postlupulone), or flavonoids (xanthohumol or xanthan humulone) with 3 mg or less of surfactant.

247. The formulation of any of embodiments 160-246, wherein the aqueous dispersion has 1 mL of liquid.

248. The formulation of any of embodiments 197-247, wherein a ratio of first LC and second LC to surfactant is at least 2:1, at least 3:1, or at least 9:1 (% w/w).

249. The formulation of any of embodiments 169-248, wherein a ratio of first LC to surfactant is at least 1:1 (% w/w).

250. The formulation of any of embodiments 197-249, wherein a ratio of first LC to second LC to surfactant is 10:8:5, 10:4:5, or 5:4:4 (% w/w).

251. The formulation of any of embodiments 197-250, wherein a ratio of first LC and second LC to glycoside is at least 1:1, 1.5:1, 2:1, 5:1, 6:1, 7:1, 8:1, 9:1, or 10:1 (% w/w).

252. The formulation of any of embodiments 160-251, wherein the aqueous dispersion has no more than 8% surfactant or no more than 5.5% w/w surfactant.

253. The formulation of any of embodiments 160-252, wherein the aqueous dispersion has no more than 2.75% w/w surfactant.

254. The formulation of embodiment 252 or 253, wherein the no more than 5.5% w/w, or 2.75% w/w surfactant has up to four surfactants.

255. The formulation of any of embodiments 252-254, wherein the no more than 5.5% w/w, or 2.75% w/w surfactant has two surfactants.

256. The formulation of any of embodiments 252-255, wherein the no more than 5.5% w/w, or 2.75% w/w surfactant has one surfactant.

257. The formulation of any of embodiments 160-256, wherein the aqueous dispersion has 10% w/w LC.

258. The formulation of any of embodiments 197-257, wherein the aqueous dispersion has 4-8% w/w second LC.

259. The formulation of any of embodiments 202-258, wherein the aqueous dispersion has 4% w/w third LC.

260. The formulation of any of embodiments 202-259, wherein the aqueous dispersion includes 10% w/w LC, 4% w/w second LC, 4% w/w third LC, and 8% or 5.5% w/w surfactant, wherein the remainder of the aqueous dispersion is water.

261. The formulation of any of embodiments 197-259, wherein the aqueous dispersion includes 10% w/w LC, 8% w/w second LC, and 8% or 5.5% w/w surfactant, wherein the remainder of the aqueous dispersion is water.

262. The formulation of any of embodiments 197-259, wherein the aqueous dispersion includes 10% w/w LC, 8% w/w second LC, and 2.75% w/w surfactant, wherein the remainder of the aqueous dispersion is water.

263. The formulation of any of embodiments 159-262, wherein the SLP includes 42.6% w/w LC, 34.0% w/w second LC, and 23.4% w/w surfactant.

264. The formulation of any of embodiments 159-262, wherein the SLP includes 38.5% w/w LC, 30.8% w/w second LC, and 30.8% w/w surfactant.

265. The formulation of any of embodiments 159-262, wherein the SLP includes 48.2% w/w LC, 38.5% w/w second LC, and 13.3% w/w surfactant.

266. The formulation of any of embodiments 159-262, wherein the SLP includes 42.6% w/w LC, 17.0% w/w second LC, 17.0% w/w third LC, and 23.4% w/w surfactant.

267. The formulation of any of embodiments 159-262, wherein the SLP includes 38.5% w/w LC, 15.4% w/w second LC, 15.4% w/w third LC, and 30.8% w/w surfactant.

268. The formulation of any of embodiments 159-262, wherein the SLP includes 42.6% w/w LC, 34% w/w third LC, and 23.4% w/w surfactant.

269. The formulation of any of embodiments 161-268, wherein the glycoside is 40% or less of the lipid particle.

270. The formulation of any of embodiments 161-269, wherein the glycoside is 30% or less of the lipid particle.

271. The formulation of any of embodiments 161-270, wherein the glycoside is 20% or less of the lipid particle.

272. The formulation of any of embodiments 159-271, wherein the % w/w surfactant includes two surfactants, three surfactants or four surfactants.

273. The formulation of any of embodiments 160-272, wherein the aqueous dispersion includes 10% w/w CBD, 8% w/w olive oil, 3.52% w/w sorbitan monooleate, 1.98% w/w polysorbate 80, and 76.5% w/w water.

274. The formulation of any of embodiments 160-272, wherein the aqueous dispersion includes 10% w/w CBD, 8% w/w olive oil, 5.12% w/w sorbitan monooleate, 2.88% w/w polysorbate 80, and 74% w/w water.

275. The formulation of any of embodiments 160-272, wherein the aqueous dispersion includes 10% w/w CBD, 8% w/w olive oil, 1.76% w/w sorbitan monooleate, 0.99% w/w polysorbate 80, and 79.25% w/w water.

276. The formulation of any of embodiments 160-272, wherein the aqueous dispersion includes 10% w/w CBD, 4% w/w olive oil, 4% w/w limonene, 3.52% w/w sorbitan monooleate, 1.98% w/w polysorbate 80, and 76.5% w/w water.

277. The formulation of any of embodiments 160-272, wherein the aqueous dispersion includes 10% w/w CBD, 4% w/w olive oil, 4% w/w limonene, 5.12% w/w sorbitan monooleate, 2.88% w/w polysorbate 80, and 74% w/w water.

278. The formulation of any of embodiments 160-272, wherein the aqueous dispersion includes 10% w/w CBD, 4% w/w olive oil, 4% bisabolol, 3.52% w/w sorbitan monooleate, 1.98% w/w polysorbate 80, and 76.5% w/w water.

279. The formulation of any of embodiments 160-272, wherein the aqueous dispersion includes 10% w/w CBD, 8% w/w bisabolol, 3.52% w/w sorbitan monooleate, 1.98% w/w polysorbate 80, and 76.5% w/w water.

280. The formulation of any of embodiments 157-279, wherein the formulation includes 10% w/w CBD, 8% w/w tocopherol, 1.76% w/w sorbitan monooleate, 0.99% w/w polysorbate 80, and 79.25% w/w water.

281. The formulation of any of embodiments 157-279, wherein the formulation includes 4.8-5.7% w/w a first LC, 0-0.1% w/w a second LC, 0.3-1% w/w surfactant, 1.1-2.1% w/w glycoside, and 90-94% w/w water.

282. The formulation of any of embodiments 157-279, wherein the formulation includes 4.8-5.8% w/w cannabidiol, 0-0.1% w/w tocopherol, 0.3-1% w/w saponin, 0.5-1% w/w rebaudioside A, 0.6-1.1% w/w mogroside V, and 90-94% w/w water.

283. The formulation of any of embodiments 157-279, wherein the formulation includes 8.8-11% w/w cannabidiol, 15-16% w/w all-rac-alpha-tocopherol, 0.3-2% w/w rebaudioside A, 0.3-2% w/w mogroside V, 1-2.7% w/w polysorbate 80, 2.2-4.5% w/w sorbitan monooleate, and 62-72% w/w water.

284. The formulation of any of embodiments 159-283, wherein the SLP includes 42.6% w/w CBD, 34.0% w/w olive oil, 15.0% w/w sorbitan monooleate, and 8.4% w/w polysorbate 80.

285. The formulation of any of embodiments 159-283, wherein the SLP includes 38.5% w/w CBD, 30.8% w/w olive oil, 19.7% w/w sorbitan monooleate, and 11.1% w/w polysorbate 80.

286. The formulation of any of embodiments 159-283, wherein the SLP includes 48.2% w/w CBD, 38.5% w/w olive oil, 8.5% w/w sorbitan monooleate, and 4.8% w/w polysorbate 80.

287. The formulation of any of embodiments 159-283, wherein the SLP includes 42.6% w/w CBD, 17.0% w/w olive oil, 17.0% w/w limonene, 15.0% w/w sorbitan monooleate, and 8.4% w/w polysorbate 80.

288. The formulation of any of embodiments 159-283, wherein the SLP includes 38.5% w/w CBD, 15.4% w/w olive oil, 15.4% w/w limonene, 19.7% w/w sorbitan monooleate, and 11.1% w/w polysorbate 80.

289. The formulation of any of embodiments 159-283, wherein the SLP includes 42.6% w/w CBD, 17.0% w/w olive oil, 17.0% w/w bisabolol, 15.0% w/w sorbitan monooleate, and 8.4% w/w polysorbate 80.

290. The formulation of any of embodiments 159-283, wherein the SLP includes 42.6% w/w CBD, 34% w/w bisabolol, 15.0% w/w sorbitan monooleate, and 8.4% w/w polysorbate 80.

291. The formulation of any of embodiments 159-283, wherein the SLP includes 48.2% w/w CBD, 38.6% w/w tocopherol, 8.5% w/w sorbitan monooleate, and 4.8% w/w polysorbate 80.

292. The formulation of any of embodiments 159-283, wherein the SLP includes 57-70% w/w of a first LC, 0.5-0.9% w/w of a second LC, 4-9% w/w surfactant, and 16-21% w/w glycoside, wherein the selected amounts do not collectively exceed 100% w/w.

293. The formulation of any of embodiments 159-283, wherein the SLP includes 62% w/w of a first LC, 0.7% w/w of a second LC, 7% w/w surfactant, and 19% w/w glycoside, wherein the selected amounts do not collectively exceed 100% w/w.

294. The formulation of any of embodiments 159-283, wherein the SLP includes 75-63% w/w cannabidiol, 0.5-1% w/w tocopherol, 5-10% w/w saponin, 7.5-11% w/w rebaudioside A, and 10-13% w/w mogroside V, wherein the selected amounts do not collectively exceed 100% w/w.

295. The formulation of any of embodiments 159-283, wherein the SLP includes 68% w/w cannabidiol, 0.8% w/w tocopherol, 8% w/w saponin, 9% w/w rebaudioside A, and 12% w/w mogroside V, wherein the selected amounts do not collectively exceed 100% w/w.

296. The formulation of any of embodiments 159-283, wherein the SLP includes 57-70% w/w cannabidiol, 0.5-1% w/w tocopherol, 4.5-9% w/w saponin, 7-10% w/w rebaudioside A, and 9-11% w/w mogroside V, wherein the selected amounts do not collectively exceed 100% w/w.

297. The formulation of any of embodiments 159-283, wherein the SLP includes 62% w/w cannabidiol, 0.7% w/w tocopherol, 7% w/w saponin, 8% w/w rebaudioside A, and 11% w/w mogroside V, wherein the selected amounts do not collectively exceed 100% w/w.

298. The formulation of any of embodiments 159-283, wherein the SLP includes 31-29% w/w cannabidiol, 54-43% w/w all-rac-alpha-tocopherol, 1-5% w/w rebaudioside A, 1-5% w/w mogroside V, 3-8% w/w polysorbate 80, and 7-12% w/w sorbitan monooleate, wherein the selected amounts do not collectively exceed 100% w/w.

299. The formulation of any of embodiments 159-283, wherein the SLP includes 30% w/w cannabidiol, 48% w/w all-rac-alpha-tocopherol, 2% w/w rebaudioside A, 2.5% w/w mogroside V, 6% w/w polysorbate 80, and 11% w/w sorbitan monooleate, wherein the selected amounts do not collectively exceed 100% w/w.

300. The formulation of any of embodiments 159-299, including at least two types of SLPs, wherein the types of SLPs have different first LCs and a common surfactant type.

301. The formulation of embodiment 300, wherein a ratio of first SLP type to second SLP type is 10:1, 9:1, 8:1, 7:1, 6:1, 5:1, 4:1, 3:1, 2:1, or 1:1.

302. The formulation of any of embodiments 159-301, including at least three types of SLPs, wherein the three types of SLPs include a first LC in the first SLP type, a second LC in the second SLP type, and a third LC in a third SLP type and a common surfactant type.

303. The formulation of embodiment 302, wherein a ratio of first SLP type to second SLP type to third SLP type is 10:1:1, 9:1:1, 8:1:1, 7:1:1, 6:1:1, 5:1:1, 4:1:1, 3:1:1, 2:1:1, 1:1:1, 10:2:1, 9:1:5, 8:4:1, 7:1:4, 6:6:1, 5:3:1, 4:1:3, 3:1:3, 2:5:1, or 1:8:1.

304. The formulation of any of embodiments 300-303, wherein the common surfactant type is an anionic surfactant type.

305. The formulation of any of embodiments 300-303, wherein the common surfactant type is a cationic surfactant type.

306. The formulation of any of embodiments 300-303, wherein the common surfactant type is a non-ionic surfactant type.

307. The formulation of any of embodiments 300-303, wherein the common surfactant type is a zwitterionic surfactant type.

308. The formulation of any of embodiments 300-303, wherein the different first LCs are different components from a same plant type.

309. The formulation of any of embodiments 300-303, wherein the different first LCs are different components from different plant types.

310. The formulation of embodiment 308, wherein a same plant type is selected from a Cannabaceae plant, a Rubiaceae plant, or a Theaceae plant.

311. The formulation of embodiment 308 or 309, wherein a same plant type is selected from a same cannabis plant species.

312. The formulation of embodiment 311, wherein the same cannabis plant species are selected from Cannabis sativa, Cannabis indica, or Cannabis ruderalis.

313. The formulation of embodiment 309, wherein the different plant types are selected from a Cannabaceae plant, a Rubiaceae plant, and a Theaceae plant.

314. The formulation of embodiment 309 or 313, wherein the different plant types are selected from a different cannabis plant species.

315. The formulation of embodiment 314, wherein the different cannabis plant species are selected from Cannabis sativa, Cannabis indica, and Cannabis ruderalis.

316. The formulation of embodiment 157, wherein the lipid particle includes 1-20% CoQ10, 0.1-2% Rebaudioside A, 0.1-2% Mogroside V, 0.1-3% Saponin, 0.005-0.5% Sodium Benzoate, and 0.005-0.5% Potassium Sorbate.

317. The formulation of embodiment 157, wherein the lipid particle includes 1-20% CBD, 1-20% α-Tocopherol, 0.01-1% Ascorbyl Palmitate, 1-10% Span 80, 0.1-2% Mogroside V, 0.1-2% Rebaudioside A, 0.1-10% Tween 80, 0.005-0.5% Sodium Benzoate, 0.005-0.5% Potassium Sorbate.

318. The formulation of embodiment 157, wherein the lipid particle includes 5-15% CoQ10, 0.2-1% Rebaudioside A, 0.2-1% Mogroside V, 0.5-2% Saponin, 0.01-0.1% Sodium Benzoate, and 0.01-0.1% Potassium Sorbate.

319. The formulation of embodiment 157, wherein the lipid particle includes 5-15% CBD, 4-12% α-Tocopherol, 0.05-0.5% Ascorbyl Palmitate, 2-6% Span 80, 0.2-1% Mogroside V, 0.2-1% Rebaudioside A, 0.5-5% Tween 80, 0.01-0.1% Sodium Benzoate, 0.01-0.1% Potassium Sorbate.

320. The formulation of embodiment 157, wherein the lipid particle includes 10% CoQ10, 0.61% Rebaudioside A, 0.82% Mogroside V, 1% Saponin, 0.03% Sodium Benzoate, and 0.04% Potassium Sorbate.

321. The formulation of embodiment 157, wherein the lipid particle includes 10% CBD, 8% α-Tocopherol, 0.1% Ascorbyl Palmitate, 3.52% Span 80, 0.82% Mogroside V, 0.62% Rebaudioside A, 1.98% Tween 80, 0.03% Sodium Benzoate, 0.04% Potassium Sorbate.

322. A beverage kit including a formulation of any of embodiments 157-321, a beverage, and a surfactant.

323. The beverage kit of embodiment 322, wherein the surfactant is within the beverage at an amount that maintains the dispersion of SLPs within the beverage following an addition of the aqueous dispersion to the beverage.

324. The beverage kit of embodiments 322 or 323, wherein the beverage is beer, coffee, or tea.

325. The beverage kit of any of embodiments 322-324, wherein the beverage is soda.

326. The beverage kit of any of embodiments 322-325, wherein the beverage is a juice-flavored drink.

327. The beverage kit of any of embodiments 322-326, wherein the beverage is carbonated water.

328. An aqueous dispersion or liquid composition including
(i) lipid particles including a first LC, a second LC, and a surfactant, and (ii) a liquid media, wherein the aqueous dispersion or liquid composition includes over 100 mg of a lipophilic plant component in 1 mL of the liquid media.

329. The aqueous dispersion or liquid composition of embodiment 328, wherein the liquid media is an aqueous media.

330. The aqueous dispersion or liquid composition of embodiment 328 or 329, further including a glycoside, wherein the glycoside is not a saponin if the surfactant is a saponin.

331. The aqueous dispersion or liquid composition of embodiment 330, wherein the lipid particle includes the glycoside.

332. The aqueous dispersion or liquid composition of embodiment 330 or 331, wherein the liquid media includes the glycoside.

333. The aqueous dispersion or liquid composition of any of embodiments 330-332 wherein the glycoside includes a terpene glycoside or saponin.

334. The aqueous dispersion or liquid composition of any of embodiments 330-333, wherein the glycoside includes a rebaudioside glycoside.

335. The aqueous dispersion or liquid composition of embodiment 334, wherein the rebaudioside glycoside includes rebaudioside A.

336. The aqueous dispersion or liquid composition of any of embodiments 330-335, wherein the glycoside includes a mogroside-type glycoside.

337. The aqueous dispersion or liquid composition of embodiment 336, wherein the mogroside-type glycoside includes mogroside V.

338. The aqueous dispersion or liquid composition of any of embodiments 328-337, wherein the first LC includes a lipophilic plant component.

339. The aqueous dispersion or liquid composition of any of embodiments 328-338, wherein the aqueous dispersion or liquid composition includes 150 mg of the first LC in 1 mL of the liquid media.

340. The aqueous dispersion or liquid composition of any of embodiments 328-339, wherein the aqueous dispersion or liquid composition includes 200 mg of the first LC in 1 mL of the liquid media.

341. The aqueous dispersion or liquid composition of any of embodiments 338-340, wherein the lipophilic plant component includes a cannabinoid, α-acids (humulone, cohumulone, adhumulone, posthumulone, prehumulone, adprehumulone), β-acids (lupulone, colupuloe, adlupulone, prelupulone, postlupulone), flavonoids (xanthohumol or xanthan humulone), tryptophan alkaloids, diterpenes, resveratrol or proanthocyanidins.

342. The aqueous dispersion or liquid composition of embodiment 341, wherein the cannabinoid includes cannabidiol (CBD), tetrahydrocannabinol (THC), tetrahydrocannabinolic acid (THCA), cannabidiolic acid (CBDA), cannabigerolic acid (CBGA), cannabichromenenic acid (CBCA), cannabigerovarinic acid (CBGVA), tetrahydrocanabivarinic acid (THCVA), cannabidivarinic acid (CBDVA), cannabichromevarinic acid (CBCVA), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethylether (CBGM), cannabielsoin (CBE), cannabicitran (CBT), or a combination thereof.

343. The aqueous dispersion or liquid composition of embodiments 341 or 342, wherein the cannabinoid includes CBD.

344. The aqueous dispersion or liquid composition of any of embodiments 341-343, wherein the cannabinoid includes CBD and THC.

345. The aqueous dispersion or liquid composition of any of embodiments 341-344, wherein the cannabinoid includes CBD and THC at a ratio of 1:1 (% w/w).

346. The aqueous dispersion or liquid composition of any of embodiments 328-345, wherein the first LC includes a synthetic and/or artificial cannabinoid.

347. The aqueous dispersion or liquid composition of any of embodiments 328-346, wherein the first LC includes a lipophilic protein or peptide.

348. The aqueous dispersion or liquid composition of any of embodiments 328-347, wherein the first LC includes a lipophilic enzyme.

349. The aqueous dispersion or liquid composition of any of embodiments 328-348, wherein the first LC includes nattokinase or bromelain.

350. The aqueous dispersion or liquid composition of any of embodiments 328-349, wherein the first LC is solid at a room temperature.

351. The aqueous dispersion or liquid composition of embodiment 350, wherein the room temperature is 20° C. to 25° C.

352. The aqueous dispersion or liquid composition of any of embodiments 328-351, wherein the surfactant includes a glycoside.

353. The aqueous dispersion or liquid composition of any of embodiments 328-352, wherein the surfactant includes a sorbitan fatty acid ester, polyethylene glycol sorbitan fatty acid ester, polyethylene-polypropylene glycol, saturated polyglycolized glyceride, polyethylene glycol, quillaia, polyethylene glycol stearate, polyethylene glycol hydrogenated castor oil, diethylene glycol monoethyl ether, propylene glycol laurate, D-α-Tocopherol polyethylene glycol 1000 succinate (TPGS), polyvinyl pyrrolidone, iota carrageenan, hydroxypropyl methylcellulose (HPMC), carboxymethyl cellulose (CMC), microcrystalline cellulose (MCC), lecithin, sucrose laurate, sucrose palmitate, sucrose stearate, gamma-cyclodextrin, β-cyclodextrin pectin, whey protein, caseinate, Xanthan gum, locust Bean gum, gellan gum, acacia gum, guar gum, Tragacanth gum, polyethylene glycol hydroxystearate polyoxyl-10-oleyl ether, or polyethylene glycol glyceride.

354. The aqueous dispersion or liquid composition of embodiment 353, wherein the polyethylene glycol sorbitan fatty acid includes polysorbate 80.

355. The aqueous dispersion or liquid composition of embodiment 353, wherein the sorbitan fatty acid ester includes sorbitan monooleate.

356. The aqueous dispersion or liquid composition of any of embodiments 328-355, including two or more surfactants.

357. The aqueous dispersion or liquid composition of any of embodiments 328-356, wherein a ratio of first LC and second LC to surfactant is greater than 9:4, 2:1, 10:8, 14:8, 28:11, 36:11, or 72:11 (% w/w).

358. The aqueous dispersion or liquid composition of any of embodiments 328-357, wherein a ratio of first LC to surfactant is 9:4, 20:5.5, 2:1, 20:11, 20:12, 20:13, 20:14, 20:15, 5:4, 10:7, or 1:1 (% w/w).

359. The aqueous dispersion or liquid composition of any of embodiments 328-358, wherein a ratio of first LC to second LC to surfactant is 40:32:11, 20:16:11, or 5:4:4 (% w/w).

360. The aqueous dispersion or liquid composition of any of embodiments 330-359, wherein a ratio of first LC and second LC to glycoside is at least 1:1, 1.5:1, 2:1, 5:1, 6:1, 7:1, 8:1, 9:1, or 10:1 (% w/w).

361. The aqueous dispersion or liquid composition of any of embodiments 328-360, wherein the first LC includes 35% w/w, 37% w/w, 40% w/w, 43% w/w, 45% w/w, 48% w/w, 50% w/w, 55% w/w, 56% w/w. 60% w/w, 63% w/w, 65% w/w, 70% w/w, 74% w/w, 75% w/w, 80% w/w, or 85% w/w of the lipid particle.

362. The aqueous dispersion or liquid composition of any of embodiments 328-360, wherein the first LC includes 42.6% w/w of the lipid particle.

363. The aqueous dispersion or liquid composition of any of embodiments 328-360, wherein the first LC includes 48.2% w/w of the lipid particle.

364. The aqueous dispersion or liquid composition of any of embodiments 328-363, wherein the surfactant includes less than 6% w/w, less than 5% w/w, less than 4% w/w, less than 3% w/w, less than 2% w/w, or less than 1% w/w of the aqueous dispersion or liquid composition.

365. The aqueous dispersion or liquid composition of any of embodiments 328-364, wherein the second LC includes 32% w/w, 32.5% w/w, 33% w/w, 34% w/w, 34.5% w/w, 35% w/w, 36% w/w, 36.5% w/w, 37% w/w, 38% w/w, 38.5% w/w, 39% w/w, or 40% w/w of the lipid particle.

366. The aqueous dispersion or liquid composition of any of embodiments 328-365, wherein the second LC includes a terpene, an essential oil, an alkaloid, a linoleic acid, a palmitic acid, a triacylglycerol, a terpene ester, a terpenoid ester, a diacylglycerol, a sterol ester, or a β-amyrin ester.

367. The aqueous dispersion or liquid composition of embodiment 366, wherein the terpene includes limonene, bisabolol, myrcene, linalool, caryophyllene, α-pinene, β-pinene, eucalyptol, trans-nerolido, humulene, delta 3 carene, camphene, borneol, terpineol, valencene, squalene, or geraniol.

368. The aqueous dispersion or liquid composition of embodiments 366 or 367, wherein the terpene includes limonene.

369. The aqueous dispersion or liquid composition of any of embodiments 366-368, wherein the terpene includes bisabolol.

370. The aqueous dispersion or liquid composition of any of embodiments 366-369, wherein the terpene includes limonene and bisabolol.

371. The aqueous dispersion or liquid composition of embodiment 366, wherein the essential oil includes α-pinene, β-pinene, myrcene, limonene, α-humulene, β-farnesene, β-caryophyllene, α-selinene, β-selinene, or γ-muurolene.

372. The aqueous dispersion or liquid composition of embodiment 366, wherein the alkaloid includes a lupuline.

373. The aqueous dispersion or liquid composition of any of embodiments 328-372, wherein the second LC includes an oil carrier.

374. The aqueous dispersion or liquid composition of embodiment 373, wherein the oil carrier includes olive oil, sesame oil, coconut oil, hemp oil, sunflower oil, or corn oil.

375. The aqueous dispersion or liquid composition of embodiments 373 or 374, wherein the oil carrier includes olive oil.

376. The aqueous dispersion or liquid composition of embodiment 366-375, wherein the second LC includes the terpene and further includes an oil carrier.

377. The aqueous dispersion or liquid composition of embodiment 376, wherein the terpene is limonene and the oil carrier is olive oil.

378. The aqueous dispersion or liquid composition of embodiment 376, wherein the terpene is bisabolol and the oil carrier is olive oil.

379. The aqueous dispersion or liquid composition of any of embodiments 328-378, wherein the lipid particles have an average particle size of 0.1 to 20 μm in diameter.

380. The aqueous dispersion or liquid composition of any of embodiments 328-379, wherein an average particle size is 1 to 3 μm in diameter.

381. The aqueous dispersion or liquid composition of any of embodiments 328-380, wherein the lipid particles are solid at room temperature.

382. The aqueous dispersion or liquid composition of embodiment 381, wherein room temperature is 20° C. to 25° C.

383. The aqueous dispersion or liquid composition of any of embodiments 328-382, formulated for delivery to a subject.

384. The aqueous dispersion or liquid composition of any of embodiments 328-383, wherein the aqueous dispersion or liquid composition is formulated for oral administration, mucosal administration, ocular administration, topical administration, or pharmaceutical administration.

385. The aqueous dispersion or liquid composition of any of embodiments 328-384, wherein the aqueous dispersion or liquid composition is formulated as a beverage, a beverage kit, a food product, or a consumer product.

386. The aqueous dispersion or liquid composition of embodiment 328, wherein the lipid particle includes 1-20% CoQ10, 0.1-2% Rebaudioside A, 0.1-2% Mogroside V, 0.1-3% Saponin, 0.005-0.5% Sodium Benzoate, and 0.005-0.5% Potassium Sorbate.

387. The aqueous dispersion or liquid composition of embodiment 328, wherein the lipid particle includes 1-20% CBD, 1-20% α-Tocopherol, 0.01-1% Ascorbyl Palmitate, 1-10% Span 80, 0.1-2% Mogroside V, 0.1-2% Rebaudioside A, 0.1-10% Tween 80, 0.005-0.5% Sodium Benzoate, 0.005-0.5% Potassium Sorbate.

388. The aqueous dispersion or liquid composition of embodiment 328, wherein the lipid particle includes 5-15% CoQ10, 0.2-1% Rebaudioside A, 0.2-1% Mogroside V, 0.5-2% Saponin, 0.01-0.1% Sodium Benzoate, and 0.01-0.1% Potassium Sorbate.

389. The aqueous dispersion or liquid composition of embodiment 328, wherein the lipid particle includes 5-15% CBD, 4-12% α-Tocopherol, 0.05-0.5% Ascorbyl Palmitate, 2-6% Span 80, 0.2-1% Mogroside V, 0.2-1% Rebaudioside A, 0.5-5% Tween 80, 0.01-0.1% Sodium Benzoate, 0.01-0.1% Potassium Sorbate.

390. The aqueous dispersion or liquid composition of embodiment 328, wherein the lipid particle includes 10% CoQ10, 0.61% Rebaudioside A, 0.82% Mogroside V, 1% Saponin, 0.03% Sodium Benzoate, and 0.04% Potassium Sorbate.

391. The aqueous dispersion or liquid composition of embodiment 328, wherein the lipid particle includes 10% CBD, 8% α-Tocopherol, 0.1% Ascorbyl Palmitate, 3.52% Span 80, 0.82% Mogroside V, 0.62% Rebaudioside A, 1.98% Tween 80, 0.03% Sodium Benzoate, 0.04% Potassium Sorbate.

392. An aqueous dispersion or liquid composition including
(i) lipid particles including a surfactant and at least two LCs in which one component includes an active compound, and
(ii) a liquid media;
wherein the lipid particles are dispersed in the liquid media and the aqueous dispersion or liquid composition includes over 100 mg of the active compound in 1 mL of the liquid media.

393. The aqueous dispersion or liquid composition of embodiment 392, wherein the liquid media is an aqueous media.

394. The aqueous dispersion or liquid composition of embodiment 392 or 393, further including a glycoside, wherein the glycoside is not a saponin if the surfactant is a saponin.

395. The aqueous dispersion or liquid composition of embodiment 394, wherein the lipid particles include the glycoside.

396. The aqueous dispersion or liquid composition of embodiment 394 or 395, wherein the liquid media includes the glycoside.

397. The aqueous dispersion or liquid composition of any of embodiments 394-396, wherein the glycoside includes a terpene glycoside or saponin.

398. The aqueous dispersion or liquid composition of any of embodiments 394-397, wherein the glycoside includes a rebaudioside glycoside.

399. The aqueous dispersion or liquid composition of embodiment 398, wherein the rebaudioside glycoside includes rebaudioside A.

400. The aqueous dispersion or liquid composition of any of embodiments 394-399, wherein the glycoside includes a mogroside-type glycoside.

401. The aqueous dispersion or liquid composition of embodiment 400, wherein the mogroside-type glycoside includes mogroside V.

402. The aqueous dispersion or liquid composition of any of embodiments 392-401, wherein the active compound includes a cannabinoid.

403. The aqueous dispersion or liquid composition of any of embodiments 392-402, wherein the aqueous dispersion or liquid composition is diluted with a liquid media to a total active compound concentration of less than 10 mg per mL of total liquid media.

404. The aqueous dispersion or liquid composition of any of embodiments 392-403, wherein the aqueous dispersion or liquid composition is dehydrated to produce a second aqueous dispersion or second liquid composition with a total active compound concentration greater than 1% w/w of total active compound.

405. The aqueous dispersion or liquid composition of any of embodiment 392-403, wherein the aqueous dispersion or liquid composition is dehydrated to produce a dehydrated solid with a total active compound concentration greater than 1% w/w of total active compound.

406. The aqueous dispersion or liquid composition of any of embodiment 392-403, wherein the aqueous dispersion or liquid composition is diluted with an aqueous phase to a total active compound concentration of over than 100 mg per mL of total liquid media.

407. A dry composition including the lipid particle of any of embodiments 1-138.

408. A method of preparing a dry composition including dehydrating the aqueous dispersion or liquid composition of any of embodiments 328-406.

409. A beverage including the formulation of any of embodiments 157-321, the aqueous dispersion or liquid composition of any of embodiments 328-406.

410. A topical composition capable of transdermal administration including (i) a lipid particle including a cannabidiol that is solid at room temperature and a surfactant, wherein a ratio of cannabidiol to surfactant is at least 1:1 (% w/w).

411. The topical composition of embodiment 410, further including a glycoside, wherein the glycoside is not a saponin if the surfactant is a saponin.

412. The topical composition of embodiment 411, wherein the glycoside includes a terpene glycoside or saponin.

413. The topical composition of embodiment 411 or 412, wherein the glycoside includes a rebaudioside glycoside.

414. The topical composition of embodiment 413, wherein the rebaudioside glycoside includes rebaudioside A.

415. The topical composition of any of embodiments 411-414 wherein the glycoside includes a mogroside-type glycoside.

416. The topical composition of embodiment 415, wherein the mogroside-type glycoside includes mogroside V.

417. The topical composition of any of embodiments 410-416, wherein the topical composition is a lotion.

418. The topical composition of any of embodiments 410-417, wherein the topical composition is a spray.

419. The topical composition of any of embodiments 410-418, wherein the cannabidiol is CBD.

### (xvii) Experimental Examples

### Example 1. Cannabidiol Loaded Solid Lipid Nanoparticles (CBD-SLNs)

TABLE 1
Formulation B wherein CBD:Oil phase (Carnauba wax +
Limonene) ratio -5:4; and Carnauba wax:Limonene ratio - 1:1.

| Sr. | | Concentration | Amount for 50 g batch |
|---|---|---|---|
| No. | Ingredients | (% w/w) | (g) |
| 1 | CBD Isolate | 10.00 | 5.00 |
| 2 | Carnauba wax | 4.00 | 2.00 |
| 3 | Limonene | 4.00 | 2.00 |
| 4 | Span 80 | 2.33 | 1.17 |
| 5 | Tween 80 | 3.17 | 1.59 |
| 6 | Water | 76.50 | 38.25 |

1. Take 80 mL glass beaker for preparation of aqueous phase and mark as “Aqueous phase.”
2. Place the beaker with stir bar on the balance and tare the balance (ensure balance displays zero). Weigh 1.59 g of Tween 80 in the glass beaker and record the amount added to the beaker. Amount of Tween 80 added: 1.58 g.
3. Tare the balance again after completing the step 2.2 when the beaker is still on the balance (ensure balance displays zero). Weigh 38.25 g of water in the glass beaker and record the amount added to the beaker. Amount of water added: 38.37 g
4. Transfer the beaker marked as “Aqueous phase” on magnetic stir hot plate, cover it with watch glass and set the heating temperature to 80° C.* (*Adjust the set temperature as needed to bring the aqueous phase to 75° C.).
5. When Aqueous phase from step 2.4 is heating, take 140 mL glass beaker for preparation of oil phase and mark as “Oil phase.”
6. Zero the balance (ensure balance displays zero). Place the beaker with stir bar on the balance and record the tare weight of the beaker. (Beaker tare weight: 60.39 g.
7. Tare the balance (ensure balance displays zero). Weigh 5.00 g of cannabidiol (CBD) isolate in the glass beaker and record the amount added to the beaker. Amount of CBD Isolate added: 5.00 g.
8. Tare the balance again after completing the step 7 when the beaker is still on the balance (ensure balance displays zero). Weigh 2.00 g of carnauba wax in the glass beaker and record the amount added to the beaker. Amount of carnauba wax added: 2.01 g.
9. Tare the balance again after completing the step 8 when the beaker is still on the balance (ensure balance displays zero). Weigh 2.00 g of limonene in the glass beaker and record the amount added to the beaker. Amount of limonene added: 2.01 g.
10. Tare the balance again after completing the step 9 when the beaker is still on the balance (ensure balance displays zero). Weigh 1.17 g of span 80 in the glass beaker and record the amount added to the beaker. Amount of span 80 added: 1.16 g.
11. Transfer the beaker onto magnetic stir hot plate and cover it with watch glass.
12. Note: Do not start stirring and heating of oil phase until water phase reaches around 67° C.-70° C. Once water phase reaches the temperature above 67° C., start heating of oil phase and set the heating temperature to 90° C.* (*Adjust the set temperature as needed to bring the oil phase to 70° C.).
13. Once aqueous phase reaches 90° C. and Oil phase reaches 90° C., transfer the aqueous phase into the oil phase while oil phase is in continuous stirring and heating at 90° C.
14. Immediately introduce the probe sonicator to the mixture while the mixture is in continuous stirring and heating at 90° C. and start sonication for 15 minutes at 90% amplitude.
15. Transfer the formulation into ice-bath placed on magnetic stir plate and stir the formulation a minimum of 15 minutes to such that the formulation temperature reaches room temperature.
16. Zero the balance (ensure balance displays zero). Turn off the magnetic stir plate and transfer the beaker to the balance after wiping down the outer surfaces of beaker. Record the initial gross weight of the beaker and calculate the water loss and remaining amount of deionized water to be added to the beaker. Beaker initial gross weight: 101.27 g.
A. Beaker initial gross weight (Step 16)−Beaker tare weight (Step 6)=Amount of Formulation present

$$101.27 - 60.39 = 40.88$$

B. 50.00 g−Amount of formulation present (A)=Water loss (Amount of deionized water to add)

$$50. g - 40.88 = 9.12$$

17. Transfer the beaker on the balance and tare the balance (ensure balance displays zero). Add the amount of deionized water calculated in step 16 (B) to the beaker and record the amount added to the beaker. Amount water added: 9.12 g

Perform PSD measurement using the Horiba LA960 and the following method:
1. Select measuring method with refractive index of 1.59, component i=0.00, manual iterations=15. Enter sample description.
2. Fill fraction cell with filtered DI water, add stir bar, place inside the LA960, start stirring, and press blank.
3. Mix formulation gently and with a micropipette, quickly add 1-2 drops of final formulation to the fraction cell while stirring.
4. As soon as sample is stabilized, measure particle size

### Assembly of Manufacturing Materials

TABLE 2
Raw Materials.

| Ingredient | Manufacturer/Grade | Lot No. |
|---|---|---|
| CBD isolate | Klersun, R&D | KCICBN-21114 |
| Carnauba wax | Koster Keunen, NF | 1700708 |
| Limonene | Sigma-Aldrich, R&D | SHBL8832 |
| Span 80 | Croda, R&D | SD40135 |
| Tween 80 | Merck, NF | 623177 |
| Water | In-house DI | n/a |

TABLE 3
Manufacturing Equipment.

| | | Calibration |
|---|---|---|
| Equipment Required | Identification Number | Due Date |
| Two-place balance | 16038 | 31 Dec. 2021 |
| Magnetic stir hot plate 1 | No LEQ#; Fisher Scientific | n/a |
| Probe sonicator | No LEQ#; QSonica with ½″ | n/a |
| | 4219 probe | |

### Example 2. Exemplary Beverage Preparation Procedure

The Tangerine Jasmine Green Tea with cannabidiol (CBD) beverage is made by the following procedure:
Measure appropriate quantity (see Table 4) of water into clean stainless pot
Heat water to 160-190 degrees F. (71.1-87.8° C.). Water temp should be watched closely for brewing.
Turn off heat
Add tea and botanical components
Gently stir at first to submerge components as needed
Steep for 2-5 minutes
Pour off liquor through filter socks/strainer into second clean pot to filter large solids.
If necessary, perform fine filtration.
Add juice concentrates, flavors, and any soluble components (including CBD dispersion).
To pasteurize, heat above 191.1° C. (196° F.) with agitation and hold for at least 3 minutes.
Fill containers with tea

TABLE 4
Ingredients for Tangerine Jasmine Green Tea with CBD.

| Ingredient Name | Amount (wt. %) |
|---|---|
| Lemon Juice Conc. | 0.01-0.5 |
| Rooibos | 0.01-0.5 |
| Peppermint | 0.01-0.5 |
| Ashwaganda | 0.01-0.5 |
| Tangerine essence (natural) | 0.1-0.9 |
| CBD Particle Dispersion | 0.005-1.0 |
| Jasmine Green Tea | 0.2-5.0 |
| Apple Juice Conc. | 0.5-5.0 |
| Tangerine Conc. | 0.5-5.0 |
| Water | 98.7-81.1 |

### Example 3. Exemplary LC and Glycoside Formulations

TABLE 5
Ingredients for a formulation with CoQ10.

| Ingredient Name | Amount (wt. %) |
|---|---|
| CoQ10 | 10 |
| Rebaudioside A | 0.61 |
| Mogroside V | 0.82 |
| Saponin | 1 |
| Sodium Benzoate | 0.03 |
| Potassium Sorbate | 0.04 |

TABLE 6
Ingredients for a formulation with CBD.

| Ingredient Name | Amount (wt. %) |
|---|---|
| CBD | 10 |
| a-Tocopherol | 8 |
| Ascorbyl Palmitate | 0.1 |
| Span 80 | 3.52 |
| Mogroside V | 0.82 |
| Rebaudioside A | 0.62 |
| Tween 80 | 1.98 |
| Sodium Benzoate | 0.03 |
| Potassium Sorbate | 0.04 |

(xviii) Closing Paragraphs. Unless otherwise indicated, the practice of the present disclosure can employ conventional techniques of chemistry, organic chemistry, biochemistry, immunology, molecular biology, microbiology, and cell biology. These methods are described in, for example, Harcourt et al., Holt McDougal Modern Chemistry: Student Edition (2018); J. Karty, Organic Chemistry Principles and Mechanisms (2014); Nelson et al., Lehninger Principles of Biochemistry 5th edition (2008); Skoog et al., Fundamentals of Analytical Chemistry (8th Edition); Atkins et al., Atkins' Physical Chemistry (11th Edition); Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to obtain a claimed effect according to a relevant experimental method described in the current disclosure.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; 19% of the stated value; ±18% of the stated value; 17% of the stated value; 16% of the stated value; ±15% of the stated value; 14% of the stated value; ±13% of the stated value; 12% of the stated value; 11% of the stated value; 10% of the stated value; 9% of the stated value; 8% of the stated value; 7% of the stated value; ±6% of the stated value; 5% of the stated value; 4% of the stated value; ±3% of the stated value; 2% of the stated value; or +1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

1. A lipid particle comprising (i) at least two lipophilic components (LCs) and (ii) a surfactant, wherein at least a first LC is solid at room temperature, and at least a percentage of the surfactant is on a surface of the lipid particle.

2. The lipid particle of claim 1, further comprising a glycoside, wherein the glycoside is not a saponin if the surfactant is a saponin.

3. The lipid particle of claim 2, wherein the glycoside comprises a terpene glycoside or saponin.

4. The lipid particle of claim 2, wherein the glycoside comprises a rebaudioside glycoside.

5. The lipid particle of claim 4, wherein the rebaudioside glycoside comprises rebaudioside A.

6. The lipid particle of claim 2, wherein the glycoside comprises a mogroside-type glycoside.

7. The lipid particle of claim 6, wherein the mogroside-type glycoside comprises mogroside V.

8. The lipid particle of claim 1, comprising at least two glycosides.

9. The lipid particle of claim 1, wherein the at least two LCs are from a same plant type.

10. The lipid particle of claim 1, wherein at least one of the at least two LCs and the surfactant are from a same plant type.

11. The lipid particle of claim 1, wherein a second LC is liquid at room temperature.

12. The lipid particle of claim 1, wherein the lipid particle is a lipid nanoparticle and/or a solid lipid particle (SLP).

13. The lipid particle of claim 9, wherein the plant type is a Cannabaceae plant.

14. The lipid particle of claim 13, wherein the Cannabaceae plant is a cannabis plant.

15. The lipid particle of claim 14, wherein the cannabis plant is Cannabis sativa, Cannabis indica, or Cannabis ruderalis.

16. The lipid particle of claim 1, wherein the first LC comprises a cannabinoid.

17. The lipid particle of claim 16, wherein the cannabinoid comprises tetrahydrocannabinol (THC), cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBT), tetrahydrocannabinolic acid (THCA), tetrahydrocannabivarinic acid (THCVA), and/or mixtures thereof.

18. The lipid particle of claim 16, wherein the cannabinoid comprises CBD.

19. The lipid particle of claim 16, wherein the cannabinoid comprises CBD and THC.

20. The lipid particle of claim 16, wherein the cannabinoid comprises CBD and THC at a ratio of 1:1 (% w/w).

21.-419. (canceled)
